(19) United States
(12) Patent Application Publication
(10) Pub. No.: US 2010/0313307 A1
(43) Pub. Date: Dec. 9, 2010

(54) PROTEIN PRODUCTION AND STORAGE IN PLANTS

(75) Inventors: ELIOT M. HERMAN, CLAYTON, MO (US); MONICA A. SCHMIDT, CLAYTON, MO (US)

Correspondence Address: Dickstein Shapiro LLP, 2049 Century Park East, Suite 700, Los Angeles, CA, 90067, US

(73) Assignees: DONALD DANFORTH PLANT SCIENCE CENTER, SAINT LOUIS, MO (US); THE UNITED STATES OF AMERICA, AS REPRESENTED BY THE SECRETARY OF AGRICULTURE, WASHINGTON, DC (US)

(21) Appl. No.: 12/493,231

(22) Filed: Jun. 28, 2009

Related U.S. Application Data
(60) Provisional application No. 61/076,616, filed on Jun. 28, 2008.

(51) Int. Cl. A01H 5/00; C12N 15/82; C12N 9/00; C07K 14/415

(52) U.S. Cl. 800/288; 800/298; 800/312; 435/183; 530/350

(57) ABSTRACT

A transgenic dicotyledonous plant having a deficiency of one or more plant seed storage proteins, further having a transgenic polynucleotide construct comprising an open reading frame operably linked to a storage protein promoter and an ER signal sequence. The polynucleotide construct encodes a protein product that can accumulate at high levels in the seed. Also provided are methods of producing a heterologous protein in a plant seed.

### CROSS-REFERENCE TO RELATED APPLICATIONS

This application claims priority to U.S. Provisional Application Ser. No. 61/076,616, filed Jun. 28, 2008, which is incorporated herein by reference in its entirety.

### STATEMENT RELATING TO FEDERALLY SPONSORED RESEARCH OR DEVELOPMENT

This invention was made, in part, with United States government support under special research grant #2006-06103 awarded by the USDA and administered by the University of Missouri, Columbia and by intramural research support of the USDA Agricultural Research Service, project number #3622-210000-025-00. The United States government may have certain rights in the invention.

### NAMES AND PARTIES TO JOINT RESEARCH AGREEMENT

This invention resulted from a joint research agreement between the Donald Danforth Plant Science Center and the U.S. Government, as represented by the U.S. Department of Agriculture, Agricultural Research Service (“USDA”). Accordingly, the United States government may have certain rights in the invention.

### FIELD OF INVENTION

The present invention relates to the field of plant genetics. More specifically, the present invention relates to genetic constructs and methods of using the constructs to modify plant seeds in order to produce an enhanced quantity of a protein of interest.

### BACKGROUND OF INVENTION

Seeds provide an important source of dietary protein for humans and livestock. Certain types of seeds, such as soybean, are capable of accumulating a relatively high level of endogenous protein, making soybean a good choice for genetic modification to produce introduced proteins. Despite the availability of many molecular tools, however, the genetic modification of seeds is often constrained by an insufficient accumulation of the engineered protein. Many intracellular processes may impact the overall protein accumulation, including transcription, translation, protein assembly and folding, methylation, phosphorylation, transport, and proteolysis. Intervention in one or more of these processes can increase the amount of protein produced in genetically engineered seeds.

Introduction of a gene can cause deleterious effect on plant growth and development. Under such circumstances, the expression of the gene may need to be limited to the desired target tissue. For example, it might be necessary to express an amino acid deregulation gene in a seed-specific fashion to avoid an undesired phenotype that may affect yield or other agronomic traits.

Soybean seeds contain from 35% to 43% protein on a dry weight basis; the majority of this protein is storage protein. There are two major soybean seed storage proteins: glycinin (also known as the 115 globulins) and beta-conglycinin (also known as the 7S globulins). Together, they comprise 70 to 80% of the seed's total protein, or 25 to 35% of the seed's dry weight.

Glycinin is a large protein with a molecular weight of about 360 kDa. It is a hexamer composed of the various combinations of five major subunits identified as G1, G2, G3, G4 and G5.

Beta-conglycinin is a heterogeneous glycoprotein with a molecular weight ranging from 150 and 240 kDa. It is composed of varying combinations of three highly negatively charged subunits identified as alpha, alpha′ and beta.

Kinney and Herman (“Cosuppression of the α Subunits of beta-conglycinin in Transgenic Soybean Seeds Induces the Formation of Endoplasmic Reticulum-Derived Protein Bodies” Plant Cell 13:1165-1178 (2001)) report that transformation with a construct containing a region transcribable to a beta-conglycinin 5′ untranslated leader sequence results in a decrease of alpha and alpha′ subunits of beta-conglycinin protein Kinney and Herman note that “[t]he decrease in beta-conglycinin protein was apparently compensated by an increased accumulation of glycinin and other vacuolar proteins in the ER” leading them to speculate that “[p]erhaps by coexpressing other proteins, perhaps as a glycinin fusion protein with a cleavable spacer, it may be possible to configure soybeans to express and accumulate at high levels foreign proteins that require ER-mediated folding and processing events.” Kinney and Herman do not teach reducing beta-conglycinin expression in a soybean seed while expressing under the control of the glycinin promoter, a foreign protein fused to an ER signal peptide.

Oulmassov et al. (US Patent Application 2005/0079494) describe expression of mutated glycinin under the control of a promoter, such as a glycinin promoter. Oulmassov et al. further describe antisense mediated suppression of sequences that contain a low content of essential amino acids, yet are expressed at relatively high levels in particular tissues, such as beta-conglycinin and glycinin. Oulmassov et al. do not teach any possible consequence of reducing expression of beta-conglycinin with respect to expression and accumulation of proteins which are expressed under the control of a glycinin promoter nor do they teach any possible consequence of suppressing beta-conglycinin expression in a soybean seed while expressing under the control of the glycinin promoter, a foreign protein fused to an ER signal peptide.

Wu (US Patent Application 2007/0067871) describes providing a soybean with an increased seed beta-conglycinin content, comprising non-transgenic mutations providing a null phenotype of at least two of the glycinin subunits. Wu does not teach reducing expression of beta-conglycinin or glycinin or expressing an exogenous protein.

What is needed in the art is a method for using soybeans to produce high levels of a protein of interest, such as for food, fuel, feed, industrial enzymes, bioprocessing enzymes, vaccines, therapeutic proteins, antibodies and the like.

### SUMMARY OF INVENTION

Provided herein are methods of producing enhanced amounts of a heterologous protein of interest in a seed. In an embodiment, genetically suppressing the production of a seed protein causes the seed to rebalance its protein composition by increasing production of other proteins to maintain normal seed protein content. This effect can be combined with the use of an “allele mimic” of the genes that are upregulated to rebalance the protein content in order to drive the expression of the heterologouos protein.

In an embodiment, provided herein is a transgenic dicotyledon having a deficiency of one or more plant storage proteins and a heterologous polynucleotide having an open reading frame operably linked to a storage protein promoter and an ER signal sequence. Optionally, the heterologous polynucleotide further comprises a 5′ translational enhancer domain (TED) and/or a 3′ TED. Optionally, the heterologous polynucleotide further comprises an ER retention sequence to induce the accretion of the heterologous polynucleotide in the lumen of the ER or an ER-derived vesicle.

In another embodiment, a transgenic dicot plant is provided, having a deficiency of one or more seed storage proteins, where the deficiency results in an at least 50% reduction in endogenous seed storage protein compared to that of a wild type plant, and a heterologous polynucleotide having a seed storage protein promoter, an open reading frame having an ER signal sequence, a desired protein coding sequence, and an ER retention signal, where the open reading frame is operably linked to the seed storage protein promoter, and where the seed of the transgenic plant is capable of producing a heterologous protein at a level that is greater than 5% of the total dry weight of the seed. The heterologous polynucleotide can also have a 5′ translational enhancer domain and/or a 3′ translational enhancer domain. The ER retention sequence can induce accretion of the heterologous protein in the lumen of the ER or an ER-derived vesicle. The dicot can be, for example, a member of the Fabaceae family, and optionally Fabales order, and optionally of soya genus. The dicot can be a member of the Glycine genus, such as a soybean. The promoter can be derived, for example, from Kunitz trypsin inhibitor, soybean lectin, immunodominant soybean allergen P34 or Gly m Bd 30 k, glucose binding protein, seed maturation protein, glycinin, or conglycinin. The translational enhancer domain can be derived from Kunitz trypsin inhibitor, Soybean lectin, immunodominant soybean allergen P34 or Gly m Bd 30 k, glucose binding protein, seed maturation protein, glycinin, or conglycinin. The storage protein deficiency can be, for example, one or more of Kunitz trypsin inhibitor, soybean lectin, immunodominant soybean allergen P34 or Gly m Bd 30 k, glucose binding protein, seed maturation protein, glycinin, or conglycinin. In an embodiment, the deficiency can be due to, for example, the presence of an RNAi, an antisense, or a sense fragment of a nucleic acid encoding a seed storage protein.

The dicot seed provided herein can have, for example, more than an 75% deficiency of the seed's endogenous storage proteins. The heterologous protein can accumulate in a seed of the dicot to a level that is greater than about 2% or greater than about 4% or greater than about 5% of the seed's total dry weight. In another embodiment, a transgenic seed, or a protein obtained from the seed, is provided. The heterologous protein can be purified.

In an embodiment, the protein coding sequence encodes an enzyme or fragment thereof. The enzyme can be cellulolytic enzyme, such as a β-glucosidase, an Exoglucanase 1, an Exoglucanase II, an endoglucanase, a xylanase, a hemicellulase, a ligninase, a ligin peroxidase, or a manganese peroxidase. In an embodiment, a commercially useful enzyme composition is provided.

In an embodiment, a transgenic dicot plant is provided herein, having a deficiency of one or more endogenous plant storage proteins, where the deficiency results in an at least 50% reduction in the level of the endogenous plant storage protein compared to a wild type plant, and a heterologous polynucleotide having a gene regulatory region of a compensating protein operably linked to an open reading frame encoding a sequence having an ER signal sequence, a desired protein coding sequence, and an ER retention signal, where the seed of the transgenic dicot plant is capable of producing the heterologous protein at a level that is greater than 5% of the total dry weight of the seed.

In another embodiment, a method of stably storing an enzyme prior to use is provided herein, by storing the enzyme in a seed from a transgenic dicot plant having a deficiency of one or more plant storage proteins, where the deficiency results in an at least 50% reduction in endogenous seed protein, and a heterologous polynucleotide having a seed storage protein promoter, an open reading frame having nucleic acid encoding an ER signal sequence, an enzyme of interest, and an ER retention signal, where the open reading frame is operably linked to the seed storage protein promoter, and where the seed of the transgenic plant is capable of producing the enzyme at a level that is greater than 5% of the total dry weight of the seed, and storing the enzyme in the seed of the transgenic dicot.

In yet another embodiment, a method of producing an enhanced amount of a heterologous protein in a dicot plant is provided herein, having stably transforming a plant cell with a polynucleotide having a seed storage protein promoter, an open reading frame having an ER signal sequence, a desired protein coding sequence, and an ER retention signal, where the open reading frame is operably linked to the seed storage protein promoter, obtaining a homozygous plant line from the stably transformed plant cell, introgressing the stably transformed plant line to a plant having a deficiency in an endogenous seed storage protein, where the deficiency results in an at least 50% reduction in the endogenous seed storage protein compared to that of a wild type plant, growing the seeds of the introgressed transgenic plant, and obtaining the heterologous protein from the seeds of the introgressed transgenic plant, where the seed of the introgressed transgenic plant is capable of producing a heterologous protein at a level that is greater than 5% of the total dry weight of the seed. The deficiency in an endogenous seed storage protein can be due to, for example, the presence of an RNAi, an antisense, or a sense fragment of a nucleic acid encoding a seed storage protein.

In yet another embodiment, a method of producing an enhanced amount of a heterologous protein in a dicot plant is provided, by stably transforming a plant cell with a polynucleotide having a seed storage protein promoter, an open reading frame comprising an ER signal sequence, a desired protein coding sequence, and an ER retention signal; wherein the open reading frame is operably linked to the seed storage protein promoter; where the polynucleotide also contains an RNAi sequence that is capable of downregulation of an endogenous seed storage protein, obtaining a homozygous plant line, and growing the seeds of the plant, to obtain a heterologous protein.

### BRIEF DESCRIPTION OF THE DRAWINGS

FIG. 1 is a model of the various pathways of subcellular localization from the endoplasmic reticulum (ER) to a protein storage vacuole or a protein body (PB).

FIG. 2 is a schematic diagram showing an RNAi construct designed to suppress glycinin. Segments to both the glycinin gene and the fad2 (fatty acid desaturase) gene were included in the RNAi construct. The fad2 segment was added as an optional feature of the construct, providing a marker for additional screening.

FIG. 3 is an electron micrograph showing that cells from seed protein deficient line “SP-” plants form protein storage vacuoles (PSVs) (Panel A) that are overtly similar in size and appearance to the PSVs formed in cells from WT seeds (Panel B). PSV: protein storage vacuole; OB: oil body; AV: autophagic vacuole; ER: endoplasmic reticulum; Nucl: nucleus; G: golgi apparatus. Bar equals 1 μm.

FIG. 4 is a photograph of a 2 dimensional isoelectric focusing/sodium dodecylsulphate-polyacrylamide gene electrophoreses (IEF/SDS-PAGE) comparison between the proteome of wild type (WT) variety “Jack” and SP-seeds.

FIG. 5 is a scatter plot of large-scale transcriptome assay of SP-compared to WT variety “Jack” using an Affymetrix DNA array platform assay.

FIG. 6 is a pie chart demonstrating the changes in seed protein composition in seeds of WT (“Jack”) vs. SP-soybean lines. The percentage composition of the various seed proteins is shown.

FIG. 7 is a schematic diagram showing the details of the GFP-kdel construct. The glycinin promoter, glycinin 5′ UTR (“TED”), ER signal sequence, GFP coding sequence, the kdel ER retention signal sequence, and the glycinin 3′ UTR (“TED”) are indicated. The transcription start site, the translation start site, and the translation stop site are indicated.

FIG. 8 is a panel of photographs showing white (Panel A) and blue (Panel B) light images of whole soybean seeds of the two homozygous parental lines and the homozygous progeny of the cross. The seeds shown have been chipped to expose the cotyledon tissue (Panels A and B). Panels C and D are pseudocolored GFP images of storage parenchyma cells from GFP-kdel (GFP protein with a C-terminal KDEL ER retention tag added) in a WT background (Panel C) and GFP-kdel×βCS (Panel D) seeds. Bar equals 10 μm.

FIG. 9 is a panel of photographs of 2D IEF-PAGE separation of protein lysates from βCS seed (β-conglycinin-suppressed; Panel A), GFP-kdel seed (Panel B), and GFP-kdel×βCS seed (Panel C), and immunoblot of a replicate lysate gel (Panel D) probed for GFP. The GFP-kdel (Panels B and C) or GFP control protein spots are enclosed in the boxes as marked. Introgression of the GFP-kdel trait into the βCS line resulted in enhanced accumulation of the GFP-kdel. The identity of the GFP spots was determined by immunoreactivity on blots using a commercial monoclonal antibody probe (panel D).

FIG. 10 shows fluorescence microscopy (Panel A), 1D PAGE (Panel B), and a β-conglycinin immunoblot (Panel C) for either βCS, GFP-kdel in WT, or GFP-kdel×βCS.

FIG. 11 is a bar graph of a fluorometric analysis of GFP-kdel abundance in seed lysates prepared from either βCS, GFP-kdel in WT, or GFP-kdel×βCS seeds, and assayed using commercial GFP as a control standard.

FIG. 12 is a photograph of a 1 dimensional PAGE of fractioned seed proteins showing the processing of glycinin in WT (“Jack”), βCS, and GFP-kdel×βCS. The resulting stained gel was scanned and the relative distribution of the proglycinin fraction of the summed proglycinin, glycinin A4, glycinin acidic subunit, and glycinin basic subunit. The results show a greater than three-fold reduction of the proglycinin fraction of glycinin protein population. Molecular weight markers and storage protein isoforms are indicated.

FIG. 13 is a photograph of a 2-D gel analysis of protein production in an SP-plant (Panel A), GFP-kdel transformed in a WT background (Panel B), and an SP-plant introgressed with GFP-kdel (Panel C).

FIG. 14 is a bar graph of a fluorometric analysis of GFP-kdel abundance in seed lysates prepared from seeds from an SP-plant, a WT plant transformed with GFP-kdel, and a GFP-kdel×SP-cross. Commercial GFP was used as a control standard.

FIG. 15 is an electron micrograph demonstrating the abundance of protein bodies in the cytoplasm of late maturation seed cells of βCS×GFP-kdel plants. Panel A: The protein bodies contain a dispersed matrix and are bounded by an ER membrane. Panel B: image demonstrating the ER origin of the protein bodies. PSV; protein storage vacuole; OB=oil body. Bar equals 1 μm.

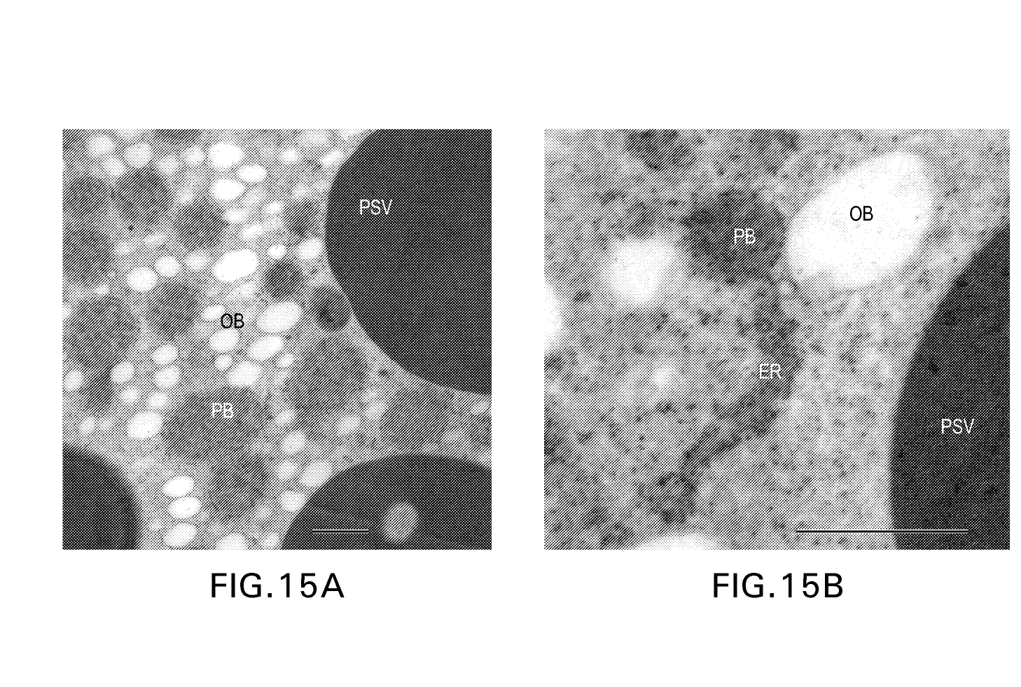

### DETAILED DESCRIPTION OF THE INVENTION

Provided herein is a genetically modified dicot plant having a seed that produces a high amount of a heterologous protein of interest. In certain embodiments, the seed is deficient in at least one endogenous seed storage protein, allowing for an enhanced amount of a foreign protein to be produced therein. The nucleic acid sequence encoding the heterologous protein can be operably linked to a regulatory region from a seed protein. In some embodiments, this regulatory region is derived from a seed protein that is naturally upregulated in response to the deficiency of the endogenous seed protein.

In certain embodiments, genetic programming in dicots can be successfully utilized to produce a protein of interest, e.g., a qualitatively and quantitatively superior protein (e.g. recombinant protein).

In certain embodiments, the genetic background of the plant is modified such that there is a deficiency in the amount of one or more storage proteins (e.g. by weight). By using one or more storage protein promoters to drive transcription of the target protein, the plant's rebalancing mechanisms(s) can result in especially high levels of a heterologous protein production.

In this embodiment, and without wishing to be bound by theory, in response to a genetic deficiency causing the loss of a major seed storage protein, the seed “rebalances” by increasing the production of other seed storage proteins. By linking a heterologous gene of interest to gene regulatory elements of an endogenous seed protein that is upregulated in order to rebalance the total amount of protein in the seed, one can produce a high level of heterologous protein in the seed. Because of this high level of accumulation of the foreign protein, this “allele mimic” method is useful for producing proteins, particularly commercially valuable proteins, in soybean or other dicot seeds.

Additionally, optional targeting of the heterologous protein to the ER allows it to stably accumulate at even higher levels in the seed. Signals can be engineered on the heterologous protein that can result in sequestration of the target protein in ER-derived vesicles, irrespective of whether the plant naturally produces protein bodies physiologically. These ER-derived vesicles are surprisingly free of other proteins, such as proteases, glycosidases, etc, yielding accumulation of higher levels of protein in a less degraded or degradable form. As used herein, the following abbreviations and definitions apply.

The term “glycinin,” refers to a major seed storage protein, also known as 11S globulin, that is present in soybean seeds.

The term “β-conglycinin” refers to a major seed storage protein, also known as 7S globulin, that is present in soybean seeds.

The term “GBP” refers to glucose binding protein.

The term “KTI” refers to Kunitz trypsin inhibitor.

The term “LE” refers to soybean lectin.

The term “P34” refers to the immunodominant soybean allergen P34 or Gly m Bd 3.

The abbreviation “fad2” refers to a sequence encoding a portion of a “fatty acid desaturase” gene.

As used herein, the term “plant” includes plant cells, plant protoplasts, plant calli, plant clumps, and plant cells that are intact in plants or parts of plants, such as pollen, flowers, embryo, seeds, pods, leaves, stems, and the like.

The term “PB” refers to a protein body. These single membrane vesicles are capable of storing proteins, and are derived directly from the endoplasmic reticulum (in contrast to the PSVs, described below).

The term “PSVs” refers to protein storage vacuoles. In seeds, these vesicles typically form from a partitioning off of the vacuole during the process of maturation and drying of the seed. Thus, protein degrading enzymes normally present in the vacuole can also be present in the PSV. In normal soybean seeds, most of the accumulated proteins are localized in these organelles.

The term “SMP” refers to a seed maturation protein

The term “storage protein” refers to a protein which specifically accumulates in a plant, e.g. in seeds.

The abbreviation “BBI” refers to “bowman birk inhibitor,” which is a serine protease inhibitor.

The term “βCS” refers to a plant that is deficient in the storage protein β-conglycinin only.

The term “SP-” refers to storage protein knockdown, that is, a plant that is deficient in both glycinin and β-conglycinin.

The term “seed” generally includes the seed proper, the seed coat and/or the seed hull, or any portion thereof.

“Seed maturation” refers to the period starting with fertilization in which metabolizable reserves, e.g., starch, sugars, oligosaccharides, phenolics, amino acids, and proteins, are deposited to various tissues in the seed, leading to seed enlargement, seed filling, and ending with seed desiccation.

The term “WT” refers to wild type and refers to a naturally occurring background of a plant, or, as apparent from the context of use, WT can refer to a plant that has a naturally occurring genetic background but for the genetic manipulation of the present invention.

The term “ORF” refers to an open reading frame; i.e. a sequence which codes for a peptide (e.g., the “target protein”). In general, this sequence is uninterrupted by introns between initiation and termination codons that encodes an amino acid sequence.

The term “heterologous polynucleotide” generally refers to a polynucleotide that does not identically exist in the host plant except as a result of a transformation event. The terms “heterologous DNA,” “heterologous gene” or “foreign DNA” refer to DNA, and typically to a DNA coding sequence (“heterologous coding sequence”), which has been introduced into plant cells from another source, that is, a non-plant source or from another species of plants, or a same-species coding sequence which is placed under the control of a plant promoter that normally controls another coding sequence.

The term “endogenous” gene refers to a native gene normally found in its natural location in the genome and is not isolated. A “foreign” gene refers to a gene not normally found in the host organism but that is introduced by gene transfer.

The term “coding sequence” or “coding region” refers to a DNA sequence that codes for a specific protein.

A “chimeric gene” or “expression cassette” in the context of the present invention, refers to a promoter sequence operably linked to DNA sequence that encodes a desired gene product, and preferably a transcription terminator sequence. In a preferred embodiment, the chimeric gene also contains a signal peptide coding region operably linked between the promoter and the gene product coding sequence in translation-frame with the gene product coding sequence. This signal sequence helps localize the protein to the ER. The sequence may further contain transcription regulatory elements, such as the above-noted transcription termination signals, as well as translation regulatory signals, such as, termination codons.

“Operably linked” refers to components of an expression cassette, being linked so as to function as a unit to express a heterologous protein. For example, a promoter operably linked to a heterologous DNA, which encodes a protein, promotes the production of functional mRNA corresponding to the heterologous DNA.

“RNA transcript” refers to the product resulting from RNA polymerase-catalyzed transcription of a DNA sequence.

The term “messenger RNA (mRNA)” generally refers to the RNA that can be translated into protein by the cell.

The term “sense” RNA generally refers to an RNA transcript that includes the mRNA. The term “antisense RNA” refers to a RNA transcript that is complementary to all or part of a target primary transcript or mRNA and that can inhibit the expression of a target gene. The complementarity of an antisense RNA may be with any part of the specific gene transcript, i.e., at the 5′ non-coding sequence, 3′ non-coding sequence, introns, or the coding sequence. The term “antisense inhibition” refers to the production of antisense RNA transcripts capable of preventing the expression of the target protein.

The term “RNAi” or “RNA interference” generally refers to methods of inhibition of expression of a protein by introducing an RNA fragment into the cell. The RNA can be encoded by a DNA fragment that is integrated into the genome. The RNAi can also be prepared by any other means known in the art. The RNAi fragment can be of any suitable length, and can be single or double stranded.

In general, “regulatory sequences” are nucleotide sequences in either endogenous or the heterologous (chimeric) genes that are located upstream (5′), within, or downstream (3′) to the protein coding region. These regulatory sequences or “regulatory regions” can regulate transcription and/or translation.

A “transcription regulatory region” or “promoter” refers to nucleic acid sequences that influence and/or promote initiation of transcription. Promoters are typically considered to include regulatory regions, such as enhancer or inducer elements.

The term “upstream regulatory regions” generally refers to a region upstream to the protein translation start codon. Thus, “upstream regulatory regions” can encompass, for example, a promoter, or it can encompass a promoter and a 5′ UTR. The upstream regulatory region can also refer to regions far upstream of the typical promoter sequence, such as “enhancer elements.”

The term “TED” refers to a translational enhancer domain.

The 5′ TED (or 5′ untranslated region (UTR)) generally refers to the region that encodes an mRNA that is 5′ (upstream) to the translational start site. Thus, the region is between the transcription start site and the translation start site. The 5′ TED can be a part of the “upstream regulatory region.”

The 3′ TED (or 3′ untranslated region (UTR)) generally refers to the region on the mRNA that is downstream of the stop codon of the protein coding sequence. This region can contain, for example, a polyadenylation signal and/or any other regulatory signal capable of affecting mRNA processing or gene expression.

“Initiation codon” and “termination codon” refer to a unit of three adjacent nucleotides in a coding sequence that specifies initiation and chain termination, respectively, of a protein sequence.

The scope of the present invention is illustrated below with various examples, optional technical features, and generic teachings.

Storage Proteins

The seeds of many plant species contain storage proteins. These proteins have been classified on the basis of their size and solubility (Higgins, T. J. (1984) Ann. Rev. Plant Physiol. 35:191-221). While not every class is found in every species, the seeds of most plant species contain proteins from more than one class. Proteins within a particular solubility or size class are generally more structurally related to members of the same class in other species than to members of a different class within the same species. In many species, the seed proteins of a given class are often encoded by multigene families, sometimes of such complexity that the families can be divided into subclasses based on sequence homology.

Soybean seeds possess a relatively high protein content, consisting largely of two storage proteins, β-conglycinin and glycinin. In wild-type seeds, β-conglycinin comprises about 15-20% of the total soybean protein. Both of these proteins are made up of multiple isoforms derived from gene families.

Pivotal storage proteins have been identified herein as being involved in a plant's programmed development. However, the normally-skilled artisan can now readily identify other storage proteins in other target plants by functional, structural, or sequence homologies. Having identified such storage proteins, knock-down experiments as described here can identify other storage proteins that are involved in protein rebalancing. Regulatory elements (e.g., promoter, TEDs, etc.) can be used to produce high levels of a desired protein according to the present invention. Thus, the many examples demonstrated herein can now be applied to other plants of potential importance to commerce or humanity.

Genetic Deficiencies

A plant of the present invention comprises a deficiency, such as for example, a genetic deficiency, resulting in a decrease of a substantial portion of the plant's endogenous seed storage protein content. For example, in various specific embodiments, the seeds of the plant can comprise less than about 75%, 70%, or less than 60% or less than about 50% or less than about 40% or less than about 25% or less than 15% of the amount of total soluble protein in the seed.

In other specific embodiments, the genetic deficiency results in an amount of a specific seed storage protein that is less than about 1%, about 2%, about 5%, about 10%, about 25%, about 50%, about 75%, or about 85% of the amount of the endogenous seed storage protein that is normally present in a WT soybean seed.

By way of example, a plant of the present invention can be deficient in one or two or three or four or more of glycinin, conglycinin, KTI, LE, P34, GBP, and SMP, or other seed storage proteins.

In an embodiment, genetic manipulation to create a deficiency of a seed storage protein can be obtained by methods such as cosuppression, antisense, RNAi, or other methods. U.S. Pat. No. 5,190,931 describes exemplary methods of the use of an antisense construct to downregulate a gene. U.S. Pat. No. 5,231,020 describes exemplary methods of the use of a sense nucleic acid construct to downregulate a gene. Genetic inhibition the expression of a gene product by use of double-stranded mRNA is disclosed in U.S. Pat. No. 6,506,559.

In other embodiments, a deficiency of a particular seed storage protein, or seed storage proteins in the aggregate, can also be attained by conventional breeding methods followed by screening for a low level of one or more seed proteins. A deficiency of a seed storage protein can also be attained by natural mutations or induced mutations, followed by screening methods to identify those plants having a low level of one or more seed storage proteins. In another embodiment, a plant having a deficiency of a seed storage protein can be obtained, for example, from a publicly available seed bank or seed repository.

A Genetic Deficiency of One Protein in the Seed Leads to Compensation (Rebalancing) by Other Seed Proteins

As described herein, the suppression of one seed protein can lead to a compensation by an increase in the production of other seed proteins, termed “compensation” or “rebalancing.” This rebalancing is demonstrated herein with plant lines having a deficiency in both glycinin and β-conglycinin (“SP-”; Example 1) and in plants having a deficiency in β-conglycinin alone (Example 11).

The suppression of the seed protein β-conglycinin by sequence mediated gene silencing was compensated for by an increased abundance of glycinin (Example 11). β-conglycinin α/α′ suppression was also achieved using RNAi technology, as also described in Example 11. This method resulted in the complete silencing of a/a′ β-conglycinin. A fraction of the increased production of glycinin was retained in the form of its precursor, proglycinin, and was sequestered in PBs. Accumulation of proteins in a protein body, instead of the PSV, demonstrates two important points: 1) that ER-derived PBs can be induced and accumulate proteins in soybean seeds and, 2) that suppression of an endogenous storage protein results in the increased accumulation of another storage protein to compensate for mass loss. This phenomenon maintains the overall protein content of the soybean seed to −40%, and is termed ‘rebalancing’.

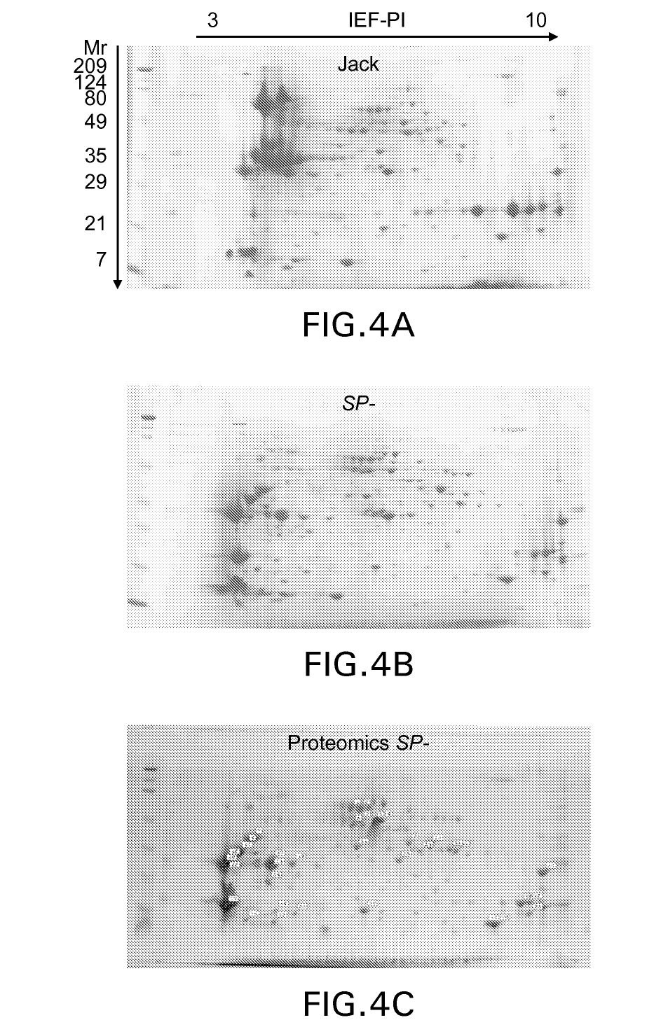

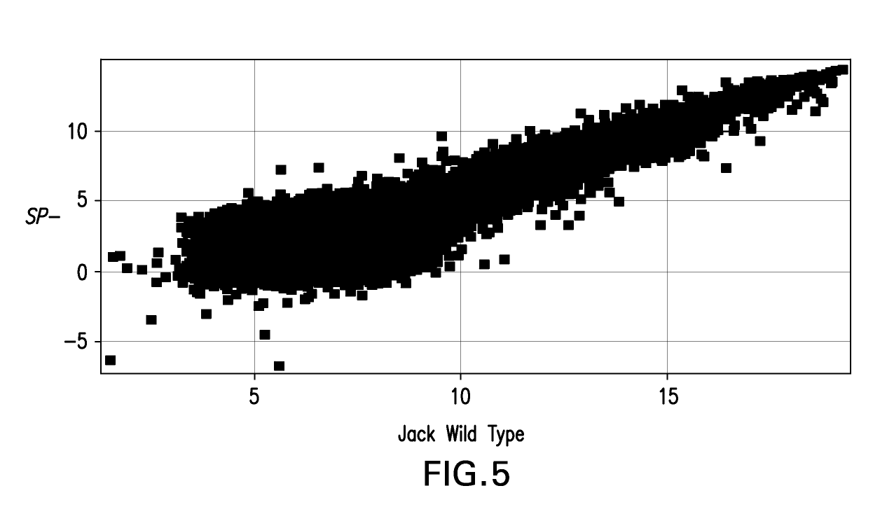

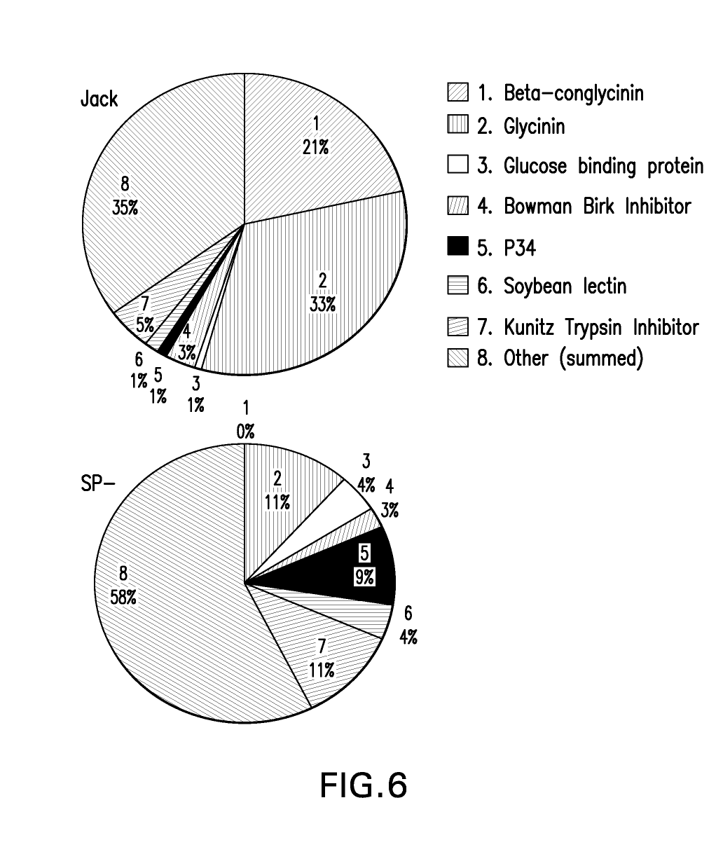

Plant lines having a deficiency of both β-conglycinin and glycinin (“SP-”) were prepared, as described in Example 1. In these seeds that were genetically deficient in both β-conglycinin and glycinin, the protein loss was compensated by the production of other proteins. The changes in protein production can be seen in FIG. 4, which is an IEF-PAGE of seed protein extracts of WT (“Jack”) compared to that of the SP-line. FIG. 5 shows the differences in the transcriptome between the two lines. FIG. 6 is a pie chart showing the percentage of several major storage proteins present in soybean seeds of WT (“Jack”) vs. the SP-line. The removal of the seed proteins β-conglycinin and glycinin in the SP-line clearly result in compensation by other proteins.

The Rebalancing Phenomenon can be Used to Produce Large Amounts of Foreign Proteins in Seeds

Provided herein are seeds that possess an intrinsic biology that may be exploited as the foundation of a protein production platform by having a foreign protein share in the rebalancing process and by accumulating the foreign protein in a stable population of PBs. Together this is the basis of developing dicot seeds as a protein production platform.

Thus, in some embodiments, one (or more) seed storage proteins is reduced as discussed above, and a desired heterologous protein is produced in the seed. In an embodiment, any suitable promoter is operably linked to the sequence encoding the heterologous protein. In another embodiment, the sequence encoding the heterologous protein is a seed-specific promoter. The promoter can be, for example, chosen from the promoters of glycinin, conglycinin, KTI, LE, P34, GBP, or SMP.

In a preferred embodiment, increased expression of the heterologous protein can occur when its gene sequence is operably linked to a promoter of the gene that encodes a protein that is upregulated in response to the above-described genetic deficiency in a soybean seed. For each specific protein that is removed from a seed, another protein (or proteins) may be produced in its place. By using the gene regulatory region (such as the promoter, terminator, and optionally other regions) of this “compensating” protein to drive the expression of the heterologous gene of interest, one can obtain an even higher level of protein production in the seed.

Accordingly, in an embodiment, the seed protein that is suppressed is β-conglycinin, while the expression of the heterologous protein is controlled by at least a portion of the regulatory region of glycinin. In an embodiment, this regulatory region is upstream of the heterologous sequence. In another embodiment, the regulatory region is downstream or 3′ of the heterologous sequence. In an embodiment the regulatory region comprises the glycinin promoter. In an embodiment the regulatory region is the glycinin upstream regulatory region, which can include, for example, the promoter and/or the 5′ UTR.

In another embodiment, the glycinin regulatory region also includes the glycinin 3′ regulatory region.

In another embodiment, the seed protein that is suppressed is both β-conglycinin and glycinin, while the heterologous protein is controlled by at least a portion of the regulatory regions (5′, 3′, or both) from one of KTI, LE, P34, GBP, or SMP.

The conceptual framework of protein rebalancing and protein sequestration in PBs was tested by constructing a transgene with the reporter protein GFP, flanked by ER transit signal sequence and retention signal sequence (KDEL), under the regulatory control of glycinin elements (Example 9). By placing the GFP-kdel construct under glycinin genetic elements, the expression of the GFP-kdel transcript will mimic that of glycinin gene expression and regulation and thereby likely participate in nutrient allocation that involves upregulation of glycinin genes. Soybeans expressing this transgene accumulated 1.6% GFP-kdel in the seed. However, when the GFP-kdel plants were genetically crossed with the βCS plants, the level of GFP-kdel expression was enhanced almost 4-fold to about 7% in the seeds (Example 12). Thus, the enhancement of GFP-kdel accumulation in the βCS seeds demonstrates that mimicking the allele of the gene participating in protein rebalancing can result in a large increase in accumulation of the heterologous protein of interest.

Thus, in embodiment, a large amount of a protein of interest can be produced in the seed. For example, the heterologous protein can be expressed from about 1%, 2%, 5%, 7%, 10%, 12%, 15%, 18%, 20%, 25%, 30%, 35%, 40%, 45%, 50%, 55%, 60%, 70%, or more of the total soluble protein in the seed.

Further, in an embodiment, the dry weight of the heterologous protein can be expressed from about 0.5%, 1%, 2%, 4%, 5%, 7%, 10%, 12%, 15%, 18%, 20%, 25%, 30%, 35%, 40%, 45%, 50% or more of the total dry weight of the seed. The heterologous protein can be produced, for example, at an amount of about 50, 100, 150, 200, 250 or more mg protein per seed.

In an embodiment, the amount of heterologous protein produced can be measured on a per plant basis. The heterologous protein can be produced, for example, at an amount of about 3 g, 6 g, 8 g, 10 g, 15 g, or 20 g or more of heterologous protein per plant.

The heterologous protein can be produced, for example, at an amount of about 25, 50, 100, 200, 300, 400, 500, 600, 700, 850, 1,000, 1,500, 2,000 or more pounds per acre per season. The actual yield can depend on many parameters such as plants per acre, plant variety, soil quality, cultivating practices, plant stress, and also the level of purity of the heterologous protein to be produced.

Promoters

In an embodiment, the heterologous polynucleotide provided herein comprises a promoter obtained from, or derived from, a plant storage protein gene. In various embodiments, the promoter is derived from the plant of the same order, family, genus or species of the plant transformed by a construct of the present invention.

Any suitable promoter can be used. In one embodiment, the promoter is a seed-specific promoter. In another embodiment, the promoter is an early seed specific promoter. In yet another embodiment, the promoter is a late seed-specific promoter. In another embodiment, the promoter is from a gene that compensates for the seed protein genetic deficiency.

The promoter sequences can end at or near the start codon and include contiguous nucleotides upstream (5′). Promoter sequences can be at least about 500 nucleotides or at least about 1000 nucleotides or at least about 1500 nucleotides. While the exact length is not critical to the invention, one skilled in the art can readily determine and optimize the promoter length (e.g. by measuring and comparing transcription levels).

In an embodiment, the upstream regulatory sequence or the promoter sequence can have a nucleic acid identity of at least 80%, 85%, 90%, 95%, 97%, 99.5%, or more to at least a portion of one of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

In a specific embodiment, the upstream regulatory region, comprising the promoter and 5′ UTR as discussed below, is chosen from SEQ ID NO: 1, SEQ ID NO: 2, SEQ ID NO: 3, SEQ ID NO: 4, SEQ ID NO: 5, SEQ ID NO: 6, SEQ ID NO: 7. SEQ ID NO: 1 and 2 are derived from glycinin, while SEQ ID NO: 3-8 represent conglycinin, KTI, LE, P34, GBP, and SMP upstream regulatory regions, respectively.

UTR Translational Enhancer Domains

A heterologous polynucleotide of the present invention optionally comprises a translational enhancer domain (TED) from a plant storage protein. In some embodiments, this is the untranslated region of the mRNA transcript. Such untranslated regions can be at the 5′ (upstream) or 3′ (downstream) region of the gene. The sequence of the TED or UTR can also be derived from another organism, or can be a completely synthetic sequence.

5′ UTR (or “5′ TED”): A construct of the present invention can comprise a 5′ TED from the 5′ region of an mRNA encoding a plant storage protein such as glycinin, conglycinin, KTI, LE, P34, GBP, or SMP. A 5′ TED can generally be identified between the promoter and the start codon of a plant storage protein. A 5′ TED can be at least about 5, 10, 25, 30, 35, 40, or more nucleotides or at least about 50 nucleotides or at least about 100 nucleotides, or at least about 150 nucleotides. While the exact length is not critical to the invention, one skilled in the art can readily determine and optimize the terminator length (e.g. by measuring and comparing translation levels). In specific embodiments, a portion of the 3′ end of the upstream regulatory sequences disclosed in SEQ ID NO: 1 (glycinin), SEQ ID NO: 2 (an alternative glycinin sequence), SEQ ID NO: 3 (conglycinin), SEQ ID NO: 4 (KTI), SEQ ID NO: 5 (LE), SEQ ID NO: 6 (P34), SEQ ID NO: 7 (GBP), and SEQ ID NO: 8 (SMP) comprise, respectively, 5′ UTR sequences.

3′UTR (or “3′ TED” or “terminator”): A TED can be derived, for example, from the 3′ region of an mRNA encoding a plant storage protein such as glycinin, conglycinin, KTI, LE, P34, GBP, or SMP. Such a 3′ TED can start at or near the stop codon and include contiguous nucleotides downstream (3′). The 3′ TED can be at least about 10, 25, 40, 50, 75, 100, 150 nucleotides or at least about 250 nucleotides or at least about 500 nucleotides. While the exact length is not critical to the invention, one skilled in the art can readily determine and optimize the terminator length (e.g. by measuring and comparing translation levels).

In an embodiment, the terminator sequence is chosen from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21. These sequences represent 3′ TED sequences for glycinin, an alternative glycinin terminator sequence, β-conglycinin, an alternative β-conglycinin terminator sequence, KTI, LE, P34, GBP, and SMP, respectively.

In an embodiment, the terminator sequence can have a nucleic acid identity of at least 80%, 85%, 90%, 95%, 97%, 99.5%, or more to one of SEQ ID NOs: 13-21.

In additional embodiments, the chimeric constructs can also comprise gene regulatory sequences that are far upstream or far downstream of the gene of interest, such as enhancer sequences. For example, transciptional “enhancer” regions can be present far upstream or downstream of a gene of interest. For example, an enhancer region can be 1,000-2,000 nucleotides or even 1 or more kb to the 5′ (upstream) end of a sequence, or can also be a present at a location of 1,000-2,000 nucleotides or even 1 or more kb downstream of the transcribed region of a gene. Thus, in some embodiments, these more distant enhancer sequences of a plant storage protein, such as glycinin, conglycinin, KTI, LE, P34, GBP, and SMP are also present in the chimeric sequence. In certain embodiments, the presence of these enhancer sequences increases the amount of the heterologous protein that is produced in the seed.

The Endoplasmic Reticulum

The endoplasmic reticulum (ER) of plants is a part of the endomembrane system, a highly conserved system in eukaryotes. Targeting proteins to the ER is of considerable interest since proteins produced in ER are trafficked to other organelles and also remain associated with the ER itself. ER-derived compartments have diverse functions, such as storage of proteins, oils, and hydrolytic enzymes used in response to pathogen attacks. Increasing knowledge of the mechanisms of storage protein trafficking to ER has lead to improvements in the use of plants as protein biofactories. Plants can store exogenous proteins within the ER in addition to other endomembrane compartments.

ER Derived Vesicles—Protein Bodies vs. Protein Storage Vacuole

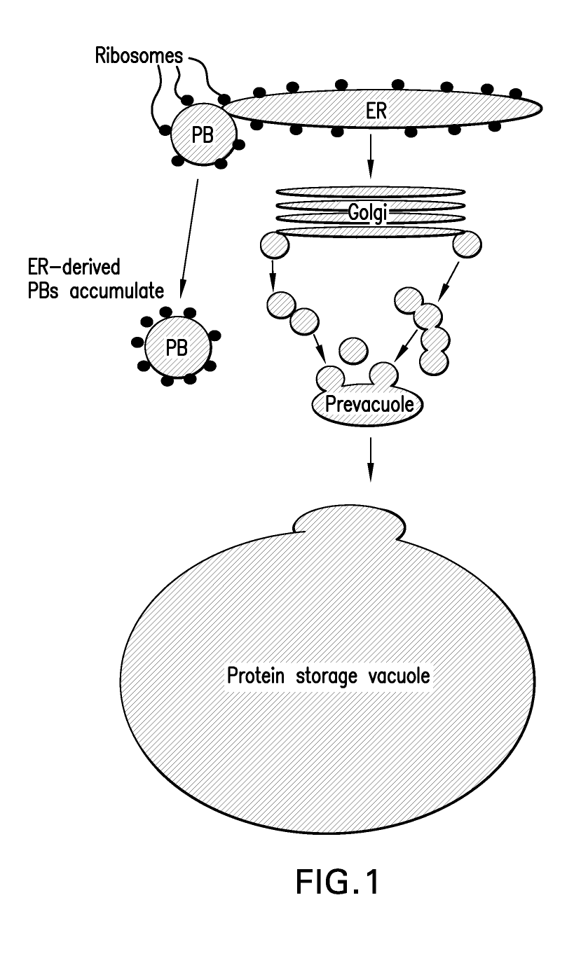

FIG. 1 shows a schematic diagram of the different subcellular protein trafficking pathways leading to the localization of a protein in a protein body or a protein storage vacuole in a soybean seed. In many plant species, but not soybean, the ER-derived PBs allow for the stable accumulation of non-glycosylated protein, because the proteins do not follow a typical endomembrane pathway from the ER to the Golgi and then on to the prevacuole.

In WT soybean plants, most seed proteins accumulate in the protein storage vacuole (PSV), instead. Proteins that are targeted to the protein storage vacuole (PSV) or to the prevacuole (which then targets to the PSV) of soybean seed are likely to be degraded quickly, however, since the vacuole typically contains many lytic enzymes.

In contrast, as disclosed herein regarding transgenic soybean seeds, the ER residence time of proteins using a C-terminal ER targeting sequence (KDEL) induces the trafficking of large amounts of foreign protein to de novo produced PBs. Thus, proteins can be sequestered in ER-derived PBs in soybean seeds. The resulting protein bodies are a stable population of organelles that persist through seed maturation and remain in the dry mature seed. This is demonstrated herein with the 27 kDa reporter protein green fluorescent protein (GFP-kdel), as shown in Example 9.

In an optional embodiment of the present invention, the heterologous sequence further comprises an ER retention sequence to induce the accretion of the heterologous polypeptide in the lumen of the ER or an ER-derived vesicle. Such ER or ER-derived vesicles include the ER, PB's, PSVs, transport vesicles, etc. Such vesicles can be identified structurally as comprising a membrane (e.g. lipid bilayer membrane), a lumen, wherein the target protein is a soluble or insoluble component residing at least partially in the lumen. Without being bound by theory, it is believed that the ER or ER-derived vesicle localization of the protein of interest is, in part, responsible for the high levels of heterologous protein produced in plants according to the present invention. In an embodiment, the heterologous polypeptide accumulates in the Golgi apparatus or Golgi vesicles.

ER Signal Sequence

In some embodiments, the heterologous polynucleotide comprises an ER signal sequence. An ER signal sequence is any polynucleotide sequence that codes for an amino acid sequence that allows for the recognition of the protein by the signal recognition particle on the endoplasmic reticulum resulting in the translocation of the protein within the ER lumen. This sequence is typically present at the N-terminal region of the protein.

In some embodiments, the ER signal sequence can be added to proteins that do not naturally have an ER targeting sequence. The heterologous protein may already have an ER signal sequence, however. If desired, this signal sequence can be replaced with another ER signal sequence, such as those shown below in Table 1. Alternatively, the protein's original signal sequence can be used. A completely synthetic signal sequence can also be used. Examples of signal sequences which direct newly synthesized proteins to the endoplasmic reticulum in plant cells include sequences from barley lectin (Dombrowski et al., 1993, Plant Cell 5:587-596), barley aleurain (Holwerda et al., 1992, Plant Cell 4:307-318), sweet potato sporamin (Matsuoka et al., 1991, Proc. Natl. Acad. Sci. USA 88:834-838), patatin (Sonnewald et al., 1991, Plant J. 1:95-106), soybean vegetative storage proteins (Mason et al., 1988, Plant Mol. Biol. 11:845-856), and beta-fructosidase (Faye et al. 1989, Plant Physiol. 89:845-851).

While the skilled artisan can readily determine useful ER signal sequences, other examples are shown in Table 1:

TABLE 1

| | |
|---|---|
| SEQ ID NO: 9 | MKIMMMIKLCFFSMSLICIAPADA |
| SEQ ID NO: 10 | MAASHGNAIFVLLLCTLFLPSLAC |
| SEQ ID NO: 11 | MAARIGIFSVFVAVLLSISAFSSA |
| SEQ ID NO: 12 | MKTNLFLFLIFSLLLSLSSAE |
| | (signal sequence from A. thaliana |
| | basic chitinase) |

Other examples of ER signal sequences are described by Emanuelsson et al (J. Mol. Biol. 300, 1005-1016 (2000)).

ER Retention Sequence

Optionally, the heterologous polynucleotide further comprises an ER retention sequence. It has been discovered that when such a sequence is added to a heterologous polynucleotide that also contains an ER signal sequence, the protein product will be retained in ER-derived vesicles where the product is sequestered from certain processing action such as proteolytic degradation. Surprisingly, the present constructs target the heterologous polynucleotide protein product to ER derived vesicles termed “protein bodies” irrespective of whether the host plant naturally produces protein bodies. Thus, it is now possible to stabilize the heterologous peptide product and to accumulate it at higher levels.

An ER retention sequence is any polynucleotide sequence that codes for an amino acid sequence known to result in the retention of a given protein at or associated with the endoplasmic reticulum such as the sequences coding for the amino acids (represented by the single letter amino acid code) KDEL (SEQ ID NO: 23), KHDEL (SEQ ID NO: 25), HDEL (SEQ ID NO: 26), KEEL (SEQ ID NO: 27) SEKDEL (SEQ ID NO: 28), and SEHDEL (SEQ ID NO: 29). Exemplary nucleic acids coding for the KDEL or KHDEL, respectively, are shown in SEQ ID NO: 22 and 24. Typically, these sequences are C-terminal in vesicular proteins and are generally 3′ in the ORF.

Alternatively, an optional ER retention sequence is derived from the C-terminal region of a vacuolar protein (wherein such sequences serve a role in delivering vacuolar proteins to plant vacuole). Non-limiting examples of such vacuolar sequences are set forth in U.S. Pat. No. 6,054,637 incorporated herein by reference. Other sequences can be readily identified by the skilled artisan by use of a functional assay.

Open Reading Frame of the Heterologous Polynucleotide to be Expressed

A heterologous polynucleotide, according to the present invention comprises an open reading frame (ORF). The ORF, coding for a protein of interest to be expressed in the seed, can be any ORF. Typically, an ORF of the present invention can code for a portion or a complete seed storage protein, fatty acid pathway enzyme, tocopherol biosynthetic enzyme, cellulosic degrading enzymes, a vaccine, a therapeutic peptide, a protein or peptide used in cosmetics, amino acid biosynthetic enzyme, or a starch branching enzyme. Typically, the ORF includes, for example, the nucleic acid encoding a target protein of interest, along with a flanking ER signal sequence at the N-terminal region, and an ER retention signal sequence at the carboxy-terminal sequence.

Optionally, the ORF is plant codon-optimized for a preferred pattern of codon usage. Modification of an ORF for optimal codon usage in plants is described in U.S. Pat. No. 5,689,052.

Choice of Protein to be Expressed

The protein of interest to be encoded by the chimeric construct can be any desired protein. The protein can be a full length protein, or can be a fragment of a full-length protein. The sequence can be derived, for example, from a plant source, an animal source, a fungal source, a viral source, a bacterial source, or it can be a completely or partially synthetic sequence.

Any desired protein can be engineered using the system described herein, regardless of its species of origin or its normal cellular location. Exemplary types of proteins that can be produced in the system described herein include but are not limited to a kinase, a structural protein, a protease, an enzyme, an amylase, a cellulolytic enzyme, an inhibitor, a protein of increased nutritional value, a pharmaceutical protein, a protein or protein fragment used in cosmetics, a protein useful for bioprocessing, a commercially useful protein, an antibody or fragment thereof, a membrane protein, a nuclear protein, a transport protein, a signaling protein, storage protein, a receptor protein, a hormone precursor, a hormone, a peptide, and a completely synthetic protein, polypeptide, or peptide sequence.

Synthetic genes encoding proteins of interest including but not limited to industrial enzymes, therapeutic enzymes and proteins, vaccines and antibodies can be inserted into the herein-described soybean seed-specific gene expression cassette that contains the 5′ and 3′ regulatory elements from glycinin, KTI, P34, SBP, SMP or LE.

In an embodiment, in order to take advantage of the protein compensation mechanism to achieve enhanced protein expression, the glycinin regulation elements can be used to drive the expression of proteins in a βCS background. In an embodiment, the regulatory elements of KTI, P34, SBP, SMP, or LE can be used to drive the expression of proteins in an SP-background. The constructs described herein can induce the proteins to participate in the protein rebalancing process resulting from the suppression of conglycinin and/or glycinin enhancing the synthesis and accumulation of proteins.

In some embodiments, the ORF has a nucleotide sequence encoding the ER-targeting signal sequence from the Arabidopsis chitinase basic gene fused 5′ and a nucleotide sequence encoding a carboxy-terminal KDEL ER retention sequence fused 3′ to the gene. Some of the proteins to be expressed possess their own intrinsic ER signal sequences; if so, these sequences may be replaced, if desired, with the ER signal sequences disclosed herein.

The plasmids can also contain the hygromycin resistance marker under the strong constitutive promoter derived from the potato ubiquitin 3 gene for selection of transformants. Transformation and production of homozygous lines containing the genes of interest can be produced as described herein. The transgenic plants can be introgressed into either the SP-, βCS, or another seed storage protein deficient line.

In an embodiment, the protein to be expressed is a cellulolytic enzyme that is useful in the biofuels industry. The biofuels industry is maturing rapidly. However, the costs for obtaining many of the enzymes needed for various biofuel production processes can be prohibitive. Obtaining the enzymes from a soybean or other dicotyledonous crop, instead, can result in lower costs associated with biofuels production, and may also be more environmentally friendly than traditional methods of obtaining such enzymes.

As an example, transgenic soybean plants as described herein can be used to create a “biofactory” to produce numerous proteins involved in cellulosic ethanol production. Thus, in an embodiment, the protein to be expressed is a cellulosic enzyme. Examples of these enzymes include but are not limited to β-glucosidase, exoglucanase 1, exoglucanase II, endoglucanase, xylanase, hemicellulase, and ligninase (such as ligin peroxidase or manganese peroxidase), and the like. The protein to be expressed can also be any other enzymes useful in the biofuels industry.

In an embodiment, the protein to be expressed is a β-glucosidase. A nucleic acid sequence or an amino acid sequence of a β-glucosidase from any species may be used. Exemplary β-glucosidases belong to the protein family EC=3.2.1.21. The sequence for this enzyme can be derived from any suitable species, such as from an Aspergillus species, for example, Aspergillus niger. Exemplary β-glucosidase nucleic acid and amino acid sequences are shown in SEQ ID NOs. 35-42.

In an embodiment, the protein to be expressed is a β-glucosidase from Aspergillus kawachii, such as shown in SEQ ID NO. 36, or a modified form of β-glucosidase from Aspergillus kawachii, such as shown in SEQ ID NOs. 37, 38, and 39. In another embodiment, the protein to be expressed is a β-glucosidase from Aspergillus niger (SEQ ID NO. 40). In yet another embodiment, the protein to be expressed is a β-glucosidase from Aspergillus terreus (e.g., XM_{—}00121222; SEQ ID NO. 42).

In an embodiment, the protein to be expressed is Exoglucanase 1, such as in the protein family EC=3.2.1.91, also known as exocellobiohydrolase I, CBH1, or 1,4-β-cellobiohydrolase. The sequence for this enzyme can be derived from any suitable source, such as, for example, Trichoderma reesei (Hypocrea jecorina). An exemplary exoglucanase I amino acid sequence is shown in SEQ ID NOs. 43 and 44.

In an embodiment, protein to be expressed is Exoglucanase II, such as in the protein family (EC=3.2.1.91), also known as exocellobiohydrolase II, CBHII, CBH2, and 1,4-β-cellobiohydrolase. The sequence for this enzyme can be derived from any suitable source, such as, for example, Trichoderma reesei (Hypocrea jecorina). An exemplary exoglucanase II amino acid sequence is shown in SEQ ID NOs. 45 and 46.

In an embodiment, protein to be expressed is an endoglucanase. Endoglucanases generally belong to the protein family EC=3.2.1.4, and are also known as endo-1,4-β-glucanase E1, cellulase E1, and endocellulase E1. The sequence for this enzyme can be derived from any suitable source, such as, for example, Acidothermus cellulolyticus. An exemplary endoglucanase amino acid sequence is shown in SEQ ID NO. 47.

In an embodiment, protein to be expressed is a xylanase. Certain xylanases, such as those belonging to the enzyme group EC 3.2.1.8 (1,4-beta-D-xylan xylanohydrolase), can catalyze the endohydrolysis of (1,4)-beta-D-xylosidic linkages in xylans. Some xylanases, such as 1,3,-beta xylanase (EC 3.2.1.32) catalyze the degradation of 1,3,-beta-D-glycosidic linkages. An exemplary xylanase nucleic acid sequence from Aspergillus niger is shown in SEQ ID NO: 48. An exemplary xylanase protein sequence from Aspergillus niger is shown in SEQ ID NO: 49.

In an embodiment, protein to be expressed is a hemicellulase. The sequence for this enzyme can be derived from any suitable source.

In an embodiment, protein to be expressed is a ligninase. These enzymes catalyze the degradation of lignin from plant cell walls. The sequence for this enzyme can be derived from any suitable source, such as, for example, a lignin-degrading basidiomycete, such as Phanerochaete chrysosporium. An exemplary ligninase amino acid sequence from Phanerochaete chrysosporium is shown in SEQ ID NO. 50.

In an embodiment, protein to be expressed is a lignase enzyme such as a manganese peroxidase (EC 1.11.1.13) enzyme. The protein sequence for this enzyme can be derived from any suitable source, such as, for example, Trametes versicolor. An exemplary manganese peroxidase amino acid sequence is shown in SEQ ID NO. 51.

Another type of lignin degrading enzyme is lignin peroxidase (for example, enzyme group EC 1.11.1.14), a hemoprotein that can catalyze the oxidative cleavage of C—C bonds and ether (C—O—C) bonds in a number of lignin compounds. This enzyme can catalyze

the following reaction: 1,2-bis(3,4-dimethoxyphenyl)propane-1,3-diol+H2O2=3,4-dimethoxybenzaldehyde+1-(3,4-dimethoxyphenyl)ethane-1,2-diol+H2O. An exemplary lignin peroxidase amino acid sequence from the microorganism Phanerochaete chrysosporium (Accession No. P49012) is shown in SEQ ID NO. 52.

In an embodiment, the protein to be expressed can have at least 80%, 85%, 90%, 95%, 97%, 99.5% identity to one of the above sequences. Alternatively, the protein to be expressed can be any other suitable protein of interest.

Methods of Stable Transformation of Plants

Nucleic acids can be incorporated into recombinant nucleic-acid constructs, typically DNA constructs, capable of being stably introduced into a plant cell.

For the practice of the present invention, conventional compositions and methods for preparing and using vectors and host cells are employed, as discussed, for example, in Sambrook et al. (eds.) (1989), Molecular Cloning: A Laboratory Manual, 2nd ed., vol. 1-3, Cold Spring Harbor Laboratory Press: Cold Spring Harbor, N.Y., and Ausubel et al., eds. (1992) Current Protocols in Molecular Biology, Greene Publishing and Wiley-Interscience, New York.

A number of vectors suitable for stable transformation of plant cells or for the establishment of transgenic plants have been described in, e.g., Pouwels et al. (1985, supp. 1987) Cloning Vectors: A Laboratory Manual; Weissbach et al., (1989) Methods for Plant Molecular Biology, Academic Press: New York; and Gelvin et al. (1990) Plant Molecular Biology Manual, Kluwer Academic Publishers. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

There are several methods of stable transformation of the construct of interest into the plant genome. Exemplary methods include, but are not limited to, microprojectile bombardment, electroporation, Agrobacterium-mediated transformation and direct DNA uptake by protoplasts.

Electroporation-based transformation methods can utilize a suspension culture of cells, embryogenic callus, or direct transformation of a tissue such as an immature embryo or other plant tissue. Protoplasts may also be employed for electroporation transformation of plants (Lazzeri et al., 1985, “A procedure for plant regeneration from immature cotyledon tissue of soybean,” Plant Mol. Biol. Rep., 3:160-167).

Transformation by Particle Bombardment

A particularly efficient method for delivering transforming DNA segments to plant cells is termed microprojectile bombardment. This method has been successfully employed for transformation of a number of plant species. In this method, particles are coated with nucleic acids and delivered into cells by a propelling force. Exemplary particles include those comprised of tungsten, platinum, or gold. For the bombardment, cells in suspension are concentrated on filters or solid culture medium. Alternatively, immature embryos or other target cells may be arranged on solid culture medium.

Many types of particle bombardment systems can be used for the transformation process. In a typical particle bombardment scenario, gold or tungsten particles are coated with the DNA construct of interest, and are placed onto a platform where a strong force (typically a gas) is used to accelerate the particles into waiting cells that have been placed so as to accept the DNA-coated particles. One exemplary system is the Biolistics Particle Delivery System (Bio-Rad Laboratories, Hercules, Calif.).

Successful transformation by particle bombardment generally requires that the target cells are actively dividing, accessible to microprojectiles, culturable in vitro, and totipotent, i.e., capable of regeneration to produce mature fertile plants. Suitable particle bombardment methods are described, for example, in U.S. Pat. No. 5,100,792, U.S. Pat. No. 5,179,022, and U.S. Pat. No. 5,204,253. Further, U.S. Pat. No. 5,015,580 describes a method of particle-mediated transformation of soybean plants by bombarding the embryonic axis from a soybean seed.

Target tissues for microprojectile bombardment can include, but are not limited to, single cells, aggregations of cells, immature embryos, young embryogenic callus from immature embryos, microspores, microspore-derived embryos, and apical meristem tissue.

In an embodiment, the transformation procedure involves particle bombardment combined with somatic embryogenesis, as described, for example, in Schmidt et al., (2008), In Vitro Cellular & Developmental Biology Plant, 44:162-168. Somatic embryogenesis is described in Bailey, 1993, In Vitro Cellular and Developmental Biology Plant 29(3): 102-108. Additional methods are described in Parrott, et al., 2004, Transgenic soybean. In: J. E. Specht and H. R. Boerma (eds). Soybeans: Improvement, Production, and Uses, 3rd Ed.-Agronomy Monograph No. 16. ASA-CSA-SSSA, Madison, Wis. pp 265-302.

Agrobacterium-mediated stable transformation can also be used to generate stably transformed plant containing the transgene of interest. The use of Agrobacterium-mediated plant integrating vectors to introduce DNA into plant cells is well known in the art (Fraley et al., 1985; U.S. Pat. No. 5,563,055). Methods of soybean transformation using Agrobacterium-based systems have been described in U.S. Pat. No. 5,569,834, the disclosure of which is specifically incorporated herein by reference in its entirety. U.S. Pat. No. 5,932,782 describes methods of transformation using Agrobacterium constructs coated onto microparticles to be used for particle bombardment. U.S. Patent Application No. US2006260012 describes methods of transforming soybean cells or tissues using Agrobacterium-based methods.

Transformation of plant protoplasts also can be achieved using various other methods, such as calcium phosphate precipitation, polyethylene glycol treatment, electroporation, and combinations of these treatments (Potrykus et al., 1985, Direct gene transfer to protoplasts: an efficient and generally applicable method for stable alterations of plant genomes, UCLA Sym Mol Cell Biol, 35:181-199). Further, plant transformation by electroporation-based gene transfer to pollen is described in U.S. Pat. No. 5,629,183.

Selectable Markers

In embodiments of the invention, a selectable marker gene is used to detect the cells that have successfully completed the transformation of the foreign gene construct. Typically, cells are screened for successful transformation within a few days to a few weeks after the transformation procedure. Screening for successful transformants can be performed most rapidly by co-transforming one or more transgene expression cassettes with a selectable marker expression cassette and conveniently by screening callus cells taken through the transformation process for a selectable marker in culture or on media plates.

The selectable marker gene in the selectable marker expression cassette is operably linked to selectable marker regulatory elements including a promoter and terminator. The expression in the transgenic plant cell of the selectable marker gene generally encodes a protein which confers resistance to an antibiotic or herbicide. Common selectable marker genes include, for example, the nptII/kanamycin resistance gene, for selection in kanamycin-containing media, the phosphinothricin acetyltransferase gene, for selection in media containing phosphinothricin (PPT), or the hph hygromycin phosphotransferase gene, for selection in media containing hygromycin B. Other selectable markers include bleomycin resistance marker genes and glufosinate resistance marker genes.

In an embodiment, the selectable marker is hygromycin phosphotransferase. An exemplary hygromycin phosphotransferase sequence is shown in SEQ ID NO: 33. In another embodiment, the selectable marker sequence is flanked by potato ubiquitin 3 upstream regulatory elements (SEQ ID NO: 30). In an additional embodiment, the selectable marker sequence is flanked by a potato ubiquitin 3 terminator sequence, such as those shown in SEQ ID NO: 31 and 32).

Regeneration of Transformed Plants

Any suitable method for regenerating the transformed plant material can be used. General methods of somatic embryogenesis are described in Parrott et al., 1994, “Somatic embryogenesis in legumes,” pp 199-227. In Y. P. S. Bajaj (ed) Biotechnology in Agriculture and Forestry. Somatic embryogenesis, Vol. 31. Springer Verlag, Berlin & Heidelberg.

Any well-known regeneration medium may be used for regenerating plants from the genetically transformed material. As used herein, “plant culture medium” refers to any medium used in the art for supporting viability and growth of a plant cell or tissue, or for growth of whole plant specimens. Such media commonly include defined components including, but not limited to: macronutrient compounds providing nutritional sources of nitrogen, phosphorus, potassium, sulfur, calcium, magnesium, and iron; micronutrients, such as boron, molybdenum, manganese, cobalt, zinc, copper, chlorine, and iodine; carbohydrates; vitamins; phytohormones; selection agents (for transformed cells or tissues, e.g., antibiotics or herbicides); and gelling agents (e.g., agar, Bactoagar, agarose, Phytagel, Gelrite, etc.). The medium may be either solid or liquid. Suitable media and regeneration methods are described, for example, in Schmidt, et al., 2005, “Towards normalization of soybean somatic embryo maturation,” Plant Cell Rep. 24:383-391.

Once the putative transformed plants are identified, the tissue can be tested to confirm that the transgene has been stably transformed into the plant genome.

The production of a homozygous line having the heterologous gene will typically require several additional crossing steps. In some embodiments, the transformed tissue is then grown into mature plants or “primary transformants” (T0), which are then self-crossed. The seeds from this self-crossing are grown (T1 generation) and positive transformants are identified and self-crossed. These seeds are then grown to mature plants (T2 generation) and screened to identify the homozygous presence of the transformed sequence, to produce a homozygous plant line.

Dicotyledons

A dicotyledon plant (or “dicot”), as provided herein, can be any dicotyledon. Optionally, the dicotyledon is a member of the Fabales order. Optionally, the dicotyledon is a member of the Fabaceae family (commonly known as legumes). Optionally, the dicot is a soybean, such as Glycine max.

Examples of dicotyledons useful in the compositions and methods provided herein are: Abrus Adans. (e.g., abrus), Acacia P. Mill. (e.g., acacia), Adenanthera L. (e.g., beadtree), Aeschynomene L. (e.g., jointvetch), Afzelia Smith (e.g., mahogany), Albizia Durazz. (e.g., albizia), Alhagi Gagnebin (e.g., alhagi), Alysicarpus Neck. ex Desv. (e.g., moneywort), Amorpha L. (e.g., false indigo, indigobush), Amphicarpa), Amphicarpaea Ell. ex Nutt. (e.g., amphicarpaea, hogpeanut), Anadenanthera Speg. (e.g., anadenanthera), Andira Juss. (e.g., andira), Anthyllis L. (e.g., kidneyvetch), Apios Fabr. (e.g., groundnut), Arachis L. (e.g., peanut), Aspalathus L. (e.g., aspalathus), Aspalthium Medik.), Astragalus L. (e.g., astragales, astragalus spp., locoweed, locoweed species, milkvetch), Baphia Lodd. (e.g., baphia), Baptisia Vent. (e.g., baptisia, False indigo, wild indigo), Barbieria D C. (e.g., barbieria), Bauhinia L. (e.g., bauhinia), Bituminaria Heister ex Fabr. (e.g., bituminaria), Bonaveria Scop.), Brongniartia Kunth (e.g., greentwig), Brya P. Br. (e.g., coccuswood), Butea Roxb. ex Willd. (e.g., butea), Caesalpinia L. (e.g., caesalpinia, nicker, poinciana), Caiandra Benth.), Cajanus Adans. (e.g., cajanus), Calliandra Benth. (e.g., calliandra, false mesquite, stickpea), Calopogonium Desv. (e.g., calopogonium), Camelina sp. (e.g., “false flax”), Canavaia Adans. Mut. Dc., Canavalia Adans. (e.g., jackbean), Caragana Fabr. (e.g., peashrub), Cassia L. (e.g., cassia, cassia species), Centrosema (D C.) Benth. (e.g., butterfly pea, centrosema), Ceratonia L. (e.g., ceratonia), Cercidium L. R. Tulasne, Cercis L. (e.g., redbud), Chamaecrista (L.) Moench (e.g., sensitive pea), Chamaecystis Link (e.g., chamaecystis), Chamaesenna (Dc.) Raf. Ex Pittier), Chapmannia Torr. & Gray (e.g., chapmannia), Christia Moench (e.g., island pea), Cicer L. (e.g., cicer), Cladrastis Raf. (e.g., yellowwood), Clitoria L. (e.g., clitoria, pigeonwings), Codariocalyx Hassk. (e.g., tick trefoil), Cojoba Britt. & Rose (e.g., cojoba), Cologania Kunth (e.g., cologania), Colutea L. (e.g., colutea), Copaifera L. (e.g., copaifera), Coronilla L. (e.g., crownvetch), Corynella D C. (e.g., corynella), Coursetia D C. (e.g., babybonnets, coursetia), Cracca Benth.), Crotalaria L. (e.g., rattlebox), Crudia Schreb.), Cullen Medik. (e.g., scurfpea), Cyamopsis D C. (e.g., cyamopsis), Cynometra L. (e.g., cynometra), Cytiscus Linnaeus), Cytisus Desf. (e.g., broom), Dalbergia L. f. (e.g., Indian rosewood), Dalea L. (e.g., dalea, dalea spp., prairie clover, prairieclover, prairieclovers), Daniellia Bennett (e.g., daniellia), Delonix Raf. (e.g., delonix), Derris Lour. (e.g., derris), Desmanthus Willd. (e.g., bundleflower), Desmodium Desv. (e.g., perennial legumes, tick trefoil, tickclover, ticktrefoil), Dialium L. (e.g., dialium), Dichrostachys (D C.) Wight & Arm. (e.g., dichrostachys), Dioclea Kunth (e.g., dioclea), Diphysa Jacq. (e.g., diphysa), Dipogon Lieb. (e.g., dipogon), Dipteryx Schreber (e.g., dipteryx), Ebenopsis Britt. & Rose (e.g., Texas ebony), Entada Adans. (e.g., callingcard vine), Enterolobium Mart. (e.g., enterolobium), Eriosema (D C.) D. Don (e.g., sand pea), Errazurizia Phil. (e.g., dunebroom), Erythrina L. (e.g., erythrina), Erythrophleum Afzel. ex R. Br. (e.g., sasswood), Eysenhardtia Kunth (e.g., kidneywood), Faidherbia A. Chev. (e.g., acacia), Falcataria (Nielsen) Barneby & Grimes (e.g., peacocksplume), Flemingia Roxb. ex Ait. f. (e.g., flemingia), Galactia P. Br. (e.g., milkpea), Galega L. (e.g., professor-weed), Genista L. (e.g., broom), Genistidium I. M. Johnston (e.g., brushpea, genistidium), Gleditsia L. (e.g., honeylocust, locust), Gliricidia Kunth (e.g., quickstick), Glottidium Desv. (e.g., glottidium), Glycine max (e.g., soybean), Glycine Willd. (e.g., soybean), Glycyrrhiza L. (e.g., licorice), Gymnocadus Lam.), Gymnocladus Lam. (e.g., coffeetree), Haematoxylum L. (e.g., haematoxylum), Halimodendron Fischer ex D C. (e.g., halimodendron), Havardia Small (e.g., havardia), Hedysarum L. (e.g., sweet vetch, sweetvetch), Hippocrepis L. (e.g., hippocrepis), Hoffmannseggia Cay. (e.g., hoffmanseggia, rushpea, rushpea species, Hoffmanseggia Cavanilles,), Hoita Rydb. (e.g., leather-root), Hymenaea L. (e.g., hymenaea), Indigofera L. (e.g., indigo), Inga P. Mill. (e.g., inga), Inocarpus J. R. & G. Forst.), Kanaloa D. H. Lorence & K. R. Wood (e.g., kanaloa), Kummerowia Schindl. (e.g., kummerowia), Lablab Adans. (e.g., lablab), Laburnum Medik. (e.g., golden chain tree), Lathyrus L. (e.g., pea, peavine, peavine spp.), Lens P. Mill. (e.g., lentil), Lespedeza Michx. (e.g., lespedeza, perennial lespedeza), Leucaena Benth. (e.g., leadtree), Lonchocarpus Kunth (e.g., lancepod), Lotononis (D C.) Ecklon & Zeyh. (e.g., lotononis), Lotus L. (e.g., deervetch, deervetch spp., trefoil), Lupinus L. (e.g., lupine, lupins), Lysiloma Benth. (e.g., false tamarind, lysiloma), Maackia Rupr. (e.g., maackia), Machaerium Pers. (e.g., machaerium), Macroptilium (Benth.) Urban (e.g., bushbean, macroptilium), Macrotyloma (Wight & Arnott) Verdc. (e.g., macrotyloma), Marina Liebm. (e.g., false prairie-clover, marina), Medicago L. (e.g., alfalfa), Meibomia Heist. Ex Fabr.), Melilotus P. Mill. (e.g., sweet clover, sweetclover), Mimosa L. (e.g., mimosa, sensitive plant), Mucuna Adans. (e.g., mucuna), Myrospermum Jacq. (e.g., myrospermum), Myroxylon L. f. (e.g., myroxylon), Neonotonia Lackey (e.g., neonotonia), Neorudolphia Britt. (e.g., neorudolphia), Neptunia Lour. (e.g., neptunia, puff), Nissolia Jacq. (e.g., nissolia, yellowhood), Olneya Gray (e.g., olneyas), Onobrychis P. Mill. (e.g., sainfoin), Ononis L. (e.g., restharrow), Orbexilum Raf. (e.g., leather-root, orbexilum), Ormosia G. Jackson (e.g., ormosia), Ornithopus L. (e.g., bird's-foot), Oxyrhynchus Brandeg. (e.g., oxyrhynchus), Oxytropis D C. (e.g., crazyweed, locoweed), Pachyrhizus L. C. Rich. ex D C. (e.g., pachyrhizus), Paraserianthes I. Nielsen (e.g., paraserianthes), Parkia R. Br. (e.g., parkia), Parkinsonia L. (e.g., paloverde, parkinsonia), Parryella Torr. & Gray ex Gray (e.g., parryella), Pediomelum Rydb. (e.g., beadroot, Indian breadroot, pediomelum, scurfpea), Peltophorum (T. Vogel) Benth. (e.g., peltophorum), Pentaclethra Benth. (e.g., pentaclethra), Pericopsis Thwaites), Peteria Gray (e.g., peteria), Phaseolus Linnaeus), Phaseolus L. (e.g., bean, wild bean), Physostigma Balf. (e.g., physostigma), Pickeringia Nutt. ex Torr. & Gray (e.g., chaparral pea), Pictetia D C. (e.g., pictetia), Piscidia L. (e.g., piscidia), Pisum L. (e.g., pea), Pitcheria Nutt.), Pithecellobium Mart. (e.g., blackbead, pithecellobium), Poitea Vent. (e.g., wattapama), Pongamia Ventenat), Prosopis L. (e.g., mesquite), Psophocarpus Necker ex D C. (e.g., psophocarpus), Psoralea Linnaeus), Psoralidium Rydb. (e.g., breadroot, scurfpea), Psorothamnus Rydb. (e.g., dalea, smokebush), Pterocarpus Jacq. (e.g., pterocarpus), Pueraria D C. (e.g., kudzu), Retama Raf., nom. cons.), Rhynchosia Lour. (e.g., snoutbean), Robinia L. (e.g., locust), Rupertia J. Grimes (e.g., rupertia), Sabinea D C. (e.g., sabinea), Samanea Merr. (e.g., raintree), Schizolobium Vogel (e.g., Brazilian firetree), Schrankia Willd. (e.g., schrankia), Scorpiurus L. (e.g., scorpion's-tail), Secula Small), Senna P. Mill. (e.g., senna), Sesbania Scop. (e.g., riverhemp, sesbania), Sophora L. (e.g., necklacepod, sophora), Spartium L. (e.g., broom), Sphaerophysa D C. (e.g., sphaerophysa), Sphenostylis E. Meyer (e.g., sphenostylis), Sphinctospermum Rose (e.g., sphinctospermum), Sphinotospermum Rose), Stahlia Bello (e.g., stahlia), Strongylodon Vogel (e.g., strongylodon), Strophostyles Ell. (e.g., fuzzy bean, fuzzybean, wildbean), Stryphnodendron C. Martius (e.g., stryphnodendron), Stylosanthes Sw. (e.g., pencilflower), Sutherlandia R. Br. (e.g., sutherlandia), Swainsonia Salisb.), Tamarindus L. (e.g., tamarind), Taralea Aublet (e.g., taralea), Tephrosia Pers. (e.g., hoarypea, tephrosia), Teramnus P. Br. (e.g., teramnus), Tetragonolobus Scop. (e.g., tetragonolobus), Thermopsis R. Br. ex Ait. f. (e.g., goldenbanner, goldenpea spp. (golden banner), thermopsis), Ticanto Adans. (e.g., gray nicker), Trifolium L. (e.g., clover, clover spp., trefles), Trigonella L. (e.g., fenugreek), Ulex L. (e.g., gorse), Vexillifera), Vicia L. (e.g., vetch, vetch spp.), Vigna Savi (e.g., cowpea, vigna), Wisteria Nutt. (e.g., wisteria), Zapoteca H. Hernandez (e.g., white stickpea), Zornia J. F. Gmel. (e.g., zornia), and the like.

Protein Processing

The proteins produced by the methods disclosed herein can be extracted from the seeds and used directly in a commercial process. Alternatively, the proteins can be partially or completely purified, then either stored or used immediately. Additionally, a soybean “meal” or grindate can be prepared from the transgenic plants, and the material can be stored until needed.

Protein Isolation, Purification, and Analysis

Protein levels can be assayed using standard proteomic procedures. Total protein content can be determined, for example, using an assay based on the Bradford method (Bradford, 1976, Analytical Biochem., 72:248-254).

Protein analysis can proceed according to widely known methods. Protein identity can be confirmed, for example, by use of gel-based assays, immunoblots, and mass spectrometry. The size of the protein can be determined, for example, using high-performance liquid chromatography.

Enzymatic activity of a specific mass of crude material or of purified enzyme can be performed using standard protocols for assaying the enzyme of interest. Measurements of enzyme stability, temperature profiles, pH profiles, and useful half-life of the enzyme can also be determined using standard methods.

The transgenic protein of interest can be purified to any extent that is required for further use. The degree of purification required can depend on many parameters, such as cost, stability of the purified protein vs. the protein remaining in the seed, downstream requirements, removal of contaminants, requirement for further processing of the protein (i.e., proper protein folding or post translational modifications), etc. An example of a method of isolating and purifying a protein produced in soybean seeds is shown in Example 16.

In embodiments of the invention, the soybean seeds can be processed, for example, by grinding or milling. A crude extract of the milled material can be prepared by adding a liquid and stirring for a time, followed by optional filtration to remove the large particles. Alternatively, in some embodiments, it may not be necessary to purify the protein of interest at all—the seed grindate containing the protein of interest, along with the rest of the soybean seed, can simply be used.

Enzyme Linked Immunosorbant Assay (ELISA)

In some embodiments, and ELISA method which is generally known in the art, can be used for analysis of the expressed protein. Using the production of beta-glucosidase in soybean as an example, the following scenario can be used. Multi-well plates are coated with rabbit anti-beta-glucosidase antibody, then the soybean seed extract and control samples are added to individual wells of the plate and incubated for 1 hour at 35° C. Anti-rabbit horseradish peroxidase conjugate is then added to each well and incubated for 1 hour at 35° C., followed by addition of the tetramethylbenzidine substrate (Sigma, USA) and incubation for 3 minutes at room temperature. The reaction is stopped by adding 1N H₂SO₄ to each well. The plates are read at 450 nm in a Microplate Reader (Bio-Rad, model 3550) and the data is processed, for example, by using MICROPLATE MANAGER™ III (Bio-Rad). The results of an analysis of several homozygous lines is measured to determine the amount of protein expressed per seed.

Storing Transgenic Proteins in the Soybean Seed

The transgenic soybean seeds disclosed herein can also be used as natural protein storage containers. Mature, dry soybean seeds containing the transgenic proteins can be stored at room temperature or below, until needed. Thus, further processing of the protein can be delayed until the time of use. This method can be an efficient and inexpensive means of storing transgenic enzymes to be used for commercial processes.

Unexpected Technical Features of the Present Invention

Surprisingly, the present invention results in a plant with superior features. For example, the invention allows for the production of either a protein of interest that naturally occurs in nature, is modified over a natural protein, is synthetic (new design), or a protein having combinations thereof. It can be produced as a protein that has desirable physicochemical properties (such as solubility or stability under various conditions; it can be produced as a fusion protein linked to residues that aid purification or enhance its utility.

The present invention is especially useful for producing proteins that otherwise are sensitive to degradation and, through sequestration by compartmentalization taught herein, can demonstrate remarkable stability.

The present invention is especially useful for producing proteins that require low humidity to preserve function. Such proteins, for example, can be targeted to ER-derived vesicles in a seed and stored for months or years and preserve nutritional value, enzymatic activity, or a desired property.

The present invention is especially useful for producing proteins that can be used commercially in unpurified form or partially purified form due to the high level of abundance in a seed or other plant part. In some embodiments, moieties can be added that provide for or prevent aggregation.

The constructs of the present invention can be combined with other useful features, such as additional regulatory elements that allow the gene to be turned on or off by temporal or external signals. Examples of useful embodiments are shown in Table 2.

TABLE 2
Exemplary Chimeric Sequences

| | | PROMOTER/ | | | | |
|---|---|---|---|---|---|---|
| | | UPSTREAM | | | | |
| | | REGULATORY | | | ER | |
| | | REGION | ER SIGNAL | | RETENTION | 3′ TED |
| PLANT | GENETICS | FROM: | SEQUENCE | ORF | SEQUENCE | FROM: |
| Soybean | Glycinin and | KTI | SEQ ID NO: 12 | Industrial | KDEL | KTI |
| | conglycinin | | | enzyme | | |
| | deficient | | | | | |
| Soybean | Glycinin, | LE | SEQ ID NO: 12 | Industrial | KDEL | LE |
| | conglycinin, | | | enzyme | | |
| | and KTI | | | | | |
| | deficient | | | | | |
| Soybean | Glycinin and | P34 | SEQ ID NO: 9 | β-glucosidase | KDEL | P34 |
| | conglycinin | | | | | |
| | deficient | | | | | |
| Soybean | B-conglycinin | glycinin | SEQ ID NO: 11 | Antibody | HDEL | glycinin |
| | deficient | | | fragment | | |
| Soybean | Glycinin | SMP | SEQ ID NO: 12 | Exoglucanase I | SEKDEL | conglycinin |
| | deficient | | | | | |
| Soybean | B-conglycinin | glycinin | SEQ ID NO: 9 | Exoglucanase II | KDEL | glycinin |
| | deficient | | | | | |
| Common | Glycinin | KTI | SEQ ID NO: 10 | Endoglucanase | HDEL | GBP |
| bean | deficient | | | | | |
| (Phaseolus | | | | | | |
| sp.) | | | | | | |
| Acacia sp. | Glycinin | KTI | SEQ ID NO: 12 | β-glucosidase | KDEL | KTI |
| | deficient | | | | | |
| Camelina sp. | Conglycinin | glycinin | SEQ ID NO: 10 | ligninase | KDEL | glycinin |
| | deficient | | | | | |

### EXAMPLES

The examples below are carried out using standard techniques, which are well known and routine to those of skill in the art, except where otherwise described in detail. The examples are illustrative, but do not limit the invention.

### Example 1

### Generation of the Storage Protein Knockdown Line “SP-”

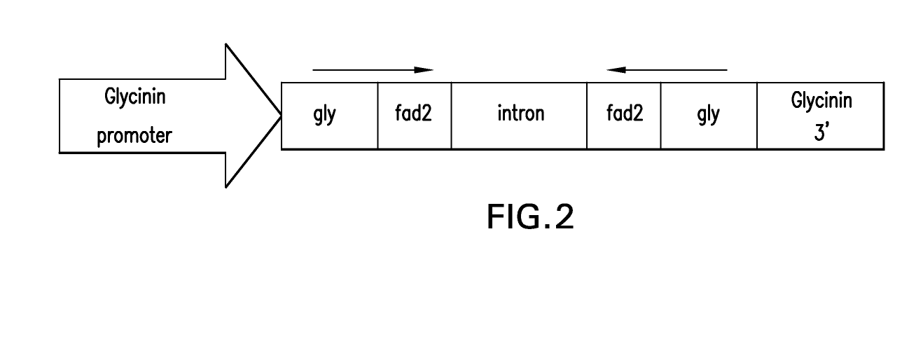

An RNAi construct designed according to FIG. 2 to suppress storage protein content in seeds was transferred to soybean using biolistic transformation protocols (Parrott, et al., 2004, “Transgenic soybean,” In: J. E. Specht and H. R. Boerma (eds). Soybeans: Improvement, Production, and Uses, 3rd Ed. Agronomy Monograph No. 16. ASA-CSA-SSSA, Madison, Wis. pp 265-302). An RNAi cassette specific for the simultaneous suppression of the endogenous soybean storage proteins and FAD2-1 omega-6 fatty acid desaturase was produced by inverting sequences specific to these open reading frames flanking an intron under the glycinin promoter and 3′ terminator. A 331 bp region of the glycinin A1bB2 gene (SEQ ID NO: 55) was placed adjacent to a 128 by region of the FAD2-1a gene (SEQ ID NO: 57). This 459 by heterologous DNA was then placed in inverted repeats about an intron. The synthetically derived intron was obtained from a portion of silencing vector p3UTR12850S. This cassette (SEQ ID NO: 56) was then placed under the regulatory elements of glycinin (FIG. 2). The FAD2 RNAi was added as an optional feature of the construct to provide a marker for additional screening potential for high-oleic phenotype and to maintain consistency with the prior conglycinin knockdown that also included the FAD2 knockdown (Kinney and Herman (“Cosuppression of the a Subunits of beta-conglycinin in Transgenic Soybean Seeds Induces the Formation of Endoplasmic Reticulum-Derived Protein Bodies,” Plant Cell 13:1165-1178 (2001).

The regenerated somatic embryos and TO seeds were screened by 1D SDS/PAGE for total protein distribution and with immunoblots assaying for cross-reactivity with anti-glycinin and anti-conglycinin antibodies.

The recovered transgenic lines not only exhibited the phenotype of suppressed glycinin content but also exhibited an essentially complete knockdown of α/α′- and β-subunits of conglycinin. Lines generated herein with a knockdown of both glycinin and conglycinin shall be referred to as SP-(storage protein knockdown).

### Example 2

### Protein and Oil Content in SP-

SP-lines were regenerated into soybean plants. The resulting plants grew and set seeds unremarkably as compared to controls. The TO seeds were chipped to assay phenotype and SP-seeds were regrown and reselected twice more to produce a homozygous population. The SP-phenotype was stable through each subsequent generation with α/α′ and β-conglycinin subunits being not detected and glycinin levels greatly reduced. The oleic acid level in the SP-seeds was >94% indicating that the FAD2 screening marker knockdown was also present.

The dry size and weight for the greenhouse grown SP-dormant seeds averaging 146 mg is similar to the wild type (WT) greenhouse grown variety “Jack” dormant seed average of 163 mg. The total protein and oil content of the SP-(40.2%, 19.1%) and the WT variety “Jack,” (37.5%, 20.5%) is similar. Thus, the assays demonstrate that the knockdown of proteins that correspond to a majority of the soybean's total protein results in the rebalancing of the soybean protein composition to a nearly identical protein/oil content and seed size.

### Example 3

### Electron Microscopy and Immunogold Immunocytochemistry

Tissue samples were cryofixed with a Balzer's high-pressure device (Bal-Tech, Principality of Liechtenstein), freeze substituted with acetone/OsO4 and embedded in epon plastic. Ultrathin sections were stained with both saturated aqueous uranyl acetate and lead citrate (33 mg/ml) prior to observation. Immunocytochemical analysis was then performed. Parallel samples were cryofixed and then processed by freeze substitution without any fixative. The substituted samples were transferred to Lowicryl HM-20 resin that was polymerized by UV light illumination. Thin sections were labeled with anti-GFP MAb (Clontech) or rabbit polyclonal anti-glycinin previously produced by this laboratory. The sections were indirected labeled with anti-IgG (rabbit or mouse)-10 nm colloidal gold (Sigma), then contrasted with 5% uranyl acetate before EM observation. All TEM was performed with a LEO 912AB microscope with imagery captured using a 2 k×2 k CCD camera operated in the montage mode.

### Example 4

### Normal Gross Morphology of SP-

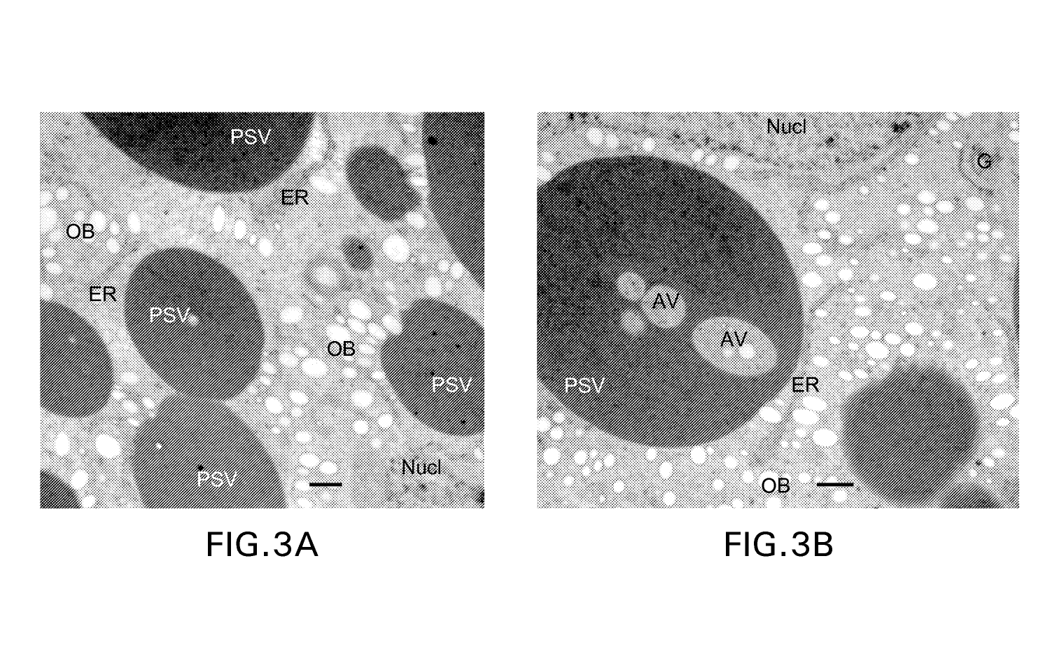

In order to examine the cellular structure of the SP- in comparison with the WT, maturing cotyledons of both were prepared by high-pressure cryofixation and the resulting samples were freeze-substituted with acetone/OsO4 and then embedded in Epon plastic, and processed as detailed above. The SP-soybeans form PSVs (FIG. 3A) that are overtly similar in size and appearance to the PSVs formed in WT seeds (FIG. 3B). The PSVs in the SP-possess a protein-filled amorphous matrix typical of soybean.

### Example 5

### Two-Dimensional Protein Analysis

Total protein was isolated from mature soybean seeds as described in Joseph et al., (2006), “Evaluation of Glycine germplasm for nulls of the immunodominant allergen P34/Gly m Bd 30 k,” Crop Science 46:1755-1763. Briefly, a total of 150 ug protein was loaded onto an 11 cm immobilized pH gradient (IPG) gel strip (pH 3-10 NL) (BioRad, Hercules Calif.) and then hydrate overnight. Isoelectric focusing (IEF) was performed for a total of 40 kVh using Protean IEF Cell (BioRad) and then run in the second dimension SDS-PAGE gel (8-16% linear gradient). Gels were stained overnight in 0.1% coomassie blue in 40% (v/v) methanol and 10% (v/v) acetic acid while blotting and subsequent immuno-detection using GFP monoclonal antibody (Clontech Inc, Mountain View, Calif.) as described in Joseph et al., (supra). Each sample was run on triplicate gels and scanned and analyzed on Phoretix 2D Evolution (version. 2005; Nonlinear Dynamics Ltd.). The GFP spots identified on the immuno-blot allowed the corresponding spots to be located on the replicate gels and the volume of these spots were normalized against the entire proteome spot volume to determine the percent volume of the GFP protein in the entire soluble soybean seed proteome.

### Example 6

### Identification of Proteins Expressed at an Increased Level Due to SP-

Proteomic analysis of the SP-soybeans show that other storage proteins compensate for the absence of storage protein polypeptides.

Two-dimensional (2D) IEF/SDS-PAGE fractionation of the SP- in comparison with the WT shows a dramatic change in the spot distribution of the proteins that results from the knockdown of the storage proteins.

FIG. 4 shows the comparison between the WT and storage protein knockdown using wide-range, pH 3-10, second dimension 2D gels. The total protein stain shows that there is a large-scale change in the protein distribution in SP- with the absent storage proteins replaced by other abundant protein spots.

The knockdown of the storage proteins was further confirmed by probing a replicate immunoblot with antibodies specific for conglycinin and glycinin storage protein fraction that showed an absence of the conglycinin subunits and isoforms and a significant reduction of the glycinin subunits and isoforms. The SP-2D gels in triplicate were evaluated by spot volume of the total proteins in comparison with the wild type. Significantly altered proteins were scored by visual examination with the assistance of gel scanning/spot volume software. Selected of protein spots were excised, subjected to tryptic fragmentation, and analyzed by tandem MS/MS mass spectroscopy. The map of the numbered protein spots selected for mass spectroscopy analysis is shown in FIG. 4C.

The compiled proteomic data from the protein spots in FIG. 4C are shown in Table 3. Much of the protein content rebalancing is due to increased content of Kunitz trypsin inhibitor (KTI), Soybean lectin (LE), also known as “agglutinin,” and the immunodominant soybean allergen P34 or Gly m Bd 30 k. Other proteins with increased content include glucose binding protein (GBP) and seed maturation protein (SMP). The proteomics and mass spectroscopy identification shows that rebalancing the shortage of storage proteins occurs by increased accumulation of only a few proteins.

TABLE 3

| Spot No. | KDal | pI | Accession No. | Protein name | % coverage | Mascot Score | No. Peptides |
|---|---|---|---|---|---|---|---|
| 1 | 60.9 | 6.42 | 170064 | Glucose binding | 37 | 604 | 22 |
| | | | | protein | | | |
| 2 | 60.9 | 6.42 | 170064 | Glucose binding | 15 | 416 | 8 |
| | | | | protein | | | |
| 3 | | | | | | | |
| 4 | 50.6 | 6.33 | 70010 | Maturation | 14 | 246 | 9 |
| | | | | protein | | | |
| 5 | 23.7 | 6.07 | 70024 | Maturation | 67 | 308 | 9 |
| | | | | associated | | | |
| | | | | protein | | | |
| 6 | 60.9 | 6.42 | 170064 | Glucose binding | 12 | 373 | 7 |
| | | | | protein | | | |
| 7 | | | | | | | |
| 8 | 40.7 | 6.19 | 22597178 | Alcohol | 20 | 278 | 10 |
| | | | | dehydrogenase | | | |
| 9 | 58.6 | 5.52 | 4249566 | glycinin | 27 | 171 | 9 |
| 10 | 40.7 | 6.19 | 22597178 | Alcohol | 38 | 395 | 17 |
| | | | | dehydrogenase | | | |
| 11 | 35.3 | 5.96 | 4102190 | 35 kDa seed | 41 | 353 | 19 |
| | | | | maturation | | | |
| | | | | protein | | | |
| 12 | 32.0 | 6.60 | 9622153 | Seed maturation | 31 | 292 | 12 |
| | | | | protein PM34 | | | |
| 13 | | | | | | | |
| 14 | 32 | 6.6 | 9622153 | Seed maturation | 35 | 274 | 13 |
| | | | | protein PM34 | | | |
| 15 | 64.4 | 5.21 | 18641 | glycinin | 20 | 291 | 7 |
| 16 | 43.1 | 5.65 | 1199563 | 34 kDa mature | 10 | 124 | 5 |
| | | | | seed vaculor | | | |
| | | | | protease | | | |
| 17 | 27.5 | 5.15 | 3114258 | agglutinin | 45 | 436 | 14 |
| 18 | | | | | | | |
| 19 | 27.6 | 5.15 | 3114258 | agglutinin | 41 | 345 | 11 |
| 20 | 64.3 | 5.21 | 18641 | Glycinin G4 | 8 | 141 | 5 |
| 21 | | | | | | | |
| 22 | 24.3 | 4.99 | 18770 | Trypsin | 17 | 141 | 5 |
| | | | | inhibitor A | | | |
| 23 | 47.1 | 8.68 | 434061 | 7S globulin | 28 | 192 | 9 |
| 24 | 27.6 | 5.15 | 3114258 | agglutinin | 28 | 225 | 9 |
| 25 | 64.4 | 5.21 | 18641 | glycinin | 10 | 204 | 6 |
| 26 | 64.4 | 5.21 | 18641 | glycinin | 19 | 353 | 12 |
| 27 | 20.3 | 4.61 | 354134 | Kunitz trypsin | 73 | 629 | 23 |
| | | | | inhibitor | | | |

FIG. 6 shows a pie chart demonstrating the amounts of various proteins in WT (“Jack”) soybean seeds vs. the SP-seeds. The chart clearly demonstrates the rebalancing or compensating process, where an increase of certain proteins occurs when other proteins are diminished in the seed.

### Example 7

### SP-Seeds Form PSV's in a Developmentally Correct Morphology and Pattern

Protein storage vacuoles (PSVs) of dicotyledonous seeds such as soybean are formed by the subdivision of the central vacuole coordinately with synthesis and deposition of the storage proteins. This results in protein-filled PSVs that fill much of the cytoplasm of seeds that are accumulating a storage protein.

The storage parenchyma cells of plants having a knockdown of both glycinin and β-conglycinin storage proteins, such as in the SP-line discussed in the above examples, appear to contain only PSV. This indicates that the compensating PSV proteins are and remain vacuolar with no redirection of the proteins into ER-derived protein bodies. The structure and distribution of all other subcellular organelles and structures appeared to be identical in the SP- and WT control.

In contrast, the single knockdown of conglycinin, whether by directed genetic engineering as shown below, for example, in Example 11, or naturally occurring mutation results in a large fraction of that glycinin remaining in the precursor proglycinin form that is accreted in ER-derived protein bodies.

### Example 8

SP-Soybean Seeds Exhibit Few Transcriptional Changes

The late maturation SP-soybeans were compared to the WT (wild type) using the Affymetrix DNA genechip with both biological and technical replicates. The resulting transcriptome data was analyzed and showed few transcripts up- or down-regulated using a relatively stringent two-fold up/down cutoff with a positive correlation ratio.

The Affymetrix genechip is based on the soybean ESTs by Shoemaker et al., 2002, A compilation of soybean ESTs: generation and analysis, Genome, 45(2):329-38, and a large fraction of these ESTs are not annotated beyond being expressed genes. Notably, among the transcripts that did not show any significant variation, were those of the proteins that did demonstrate increased protein abundance in the SP-seed proteome, namely, KTI, P34 and LE.

The transcript encoding for the major protein associated with oil bodies, oleosin, was among those to have not differed in the SP-seed. A majority of the soybean seed proteome is remodeled with little parallel consequence on transcriptome. The close similarity of the array data of the SP- and WT transcripts is illustrated in the scatter plot shown in FIG. 5.

### Example 9

GFP-KDEL Driven by the Glycinin Promoter

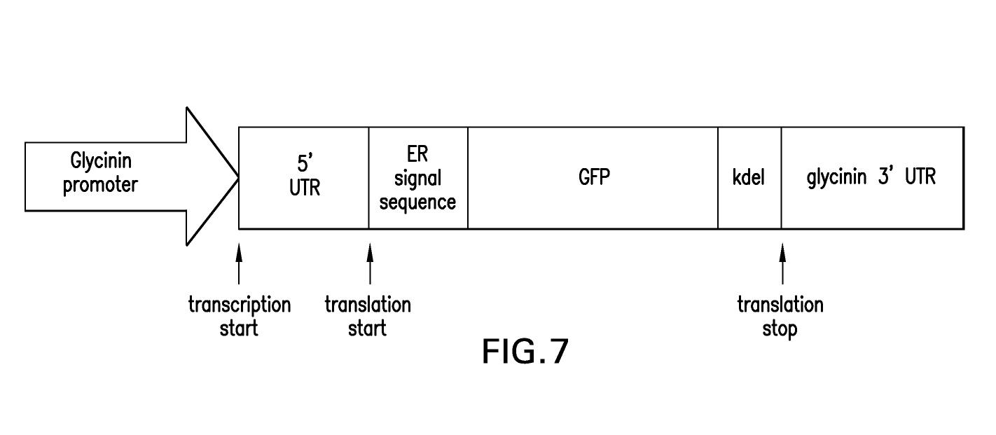

To demonstrate how the method can be used to produce high levels of protein in a seed, a construct was made having the ORF of the heterologous polynucleotide coding for GFP, along with an ER signal peptide and exemplary ER retention signal “KDEL” driven by the glycinin promoter and a 3′ TED derived from glycinin (see FIG. 7). Soybean (Glycine max WT variety “Jack”) somatic embryo transformation by biolistics was performed as described in Trick et al. 1997, “Recent advances in soybean transformation,” Plant Tissue Culture and Biology, 3(1):9-26) and regeneration as described in Schmidt et al., 2004, “Towards normalization of soybean somatic embryo maturation,” Plant Cell Reports 24:383-391. The hygromycin resistance gene under the control of the potato ubiquitin 3 regulatory elements (Garbarino and Belknap, 1994, “Isolation of a ubiquitin-ribosomal protein gene (ubi3) from potato and expression of its promoter in transgenic plants,” Plant Mol. Biol. 24(1):119-127) was used as a selectable marker in tissue culture. A commercially available GFP (Clontech Inc., Mountain View, Calif.) open reading frame (SEQ ID NO: 34), minus the start codon and stop codons, was placed into a cassette containing the seed-specific glycinin regulatory elements (Nielsen et al., 1989, “Characterization of the glycinin gene family in soybean,” Plant Cell, 1(3):313-328), a 21 amino acid ER-signal sequence from Arabidopsis chitinase gene (SEQ ID NO: 12), and a KDEL retention tag (SEQ ID NO: 23) as described in Moravec et al., 2007, “Production of Escherichia coli heat labile toxin (LT) B subunit in soybean seed and analysis of its immunogenicity as an oral vaccine,” Vaccine, 25(9):1647-57. The construct is shown in FIG. 7.

Mature dry seeds were harvested and visually observed for GFP-kdel expression under a fluorescence dissecting microscope using blue (450 nm) light for excitation. GFP-kdel positive plants were grown to the T1 generation to obtain homozygous seeds. GFP-kdel seeds were examined at the cellular level using a two-photon excitation Zeiss LSM 510 microscope with excitation at 488 nm using a 512 nm emission filter.

The construct was introduced into soybean by biolistic transformation followed by selection and regeneration of the plants. GFP-kdel positive seeds were re-grown for T1 and T2 generations producing a homozygous line of seed-specific GFP-kdel expressing seeds.

The GFP-KDEL expression in the parental homozygous seeds results in accumulation of 1.6% GFP-kdel in the soybean assayed by spot volume comparison from 2D IEF/SDS-PAGE and by assays of seed lysates using a fluorometer assay with a standard curve control using commercially obtained GFP. The fluorescent light microscopy images showed that GFP-kdel PBs are distributed throughout the cytoplasm. TEM-immunogold assays of the GFP-kdel PBs using a commercial MAb specific for GFP labeled 0.2-0.3 um diameter ER-bounded PB-like structures as previously observed.

### Example 10

### GFP-kdel Driven by the Glycinin Promoter in the SP-Line

The impact of the expression of a foreign protein was further tested in the context of the protein rebalancing process occurring in the SP-. The construct containing the GFP-kdel driven by the glycinin promoter was introduced into soybean by biolistic transformation followed by selection and regeneration of the plants, as described in Example 9. GFP-kdel positive seeds were re-grown for T1 and T2 generations producing a homozygous line of seed-specific GFP-kdel expressing seeds.

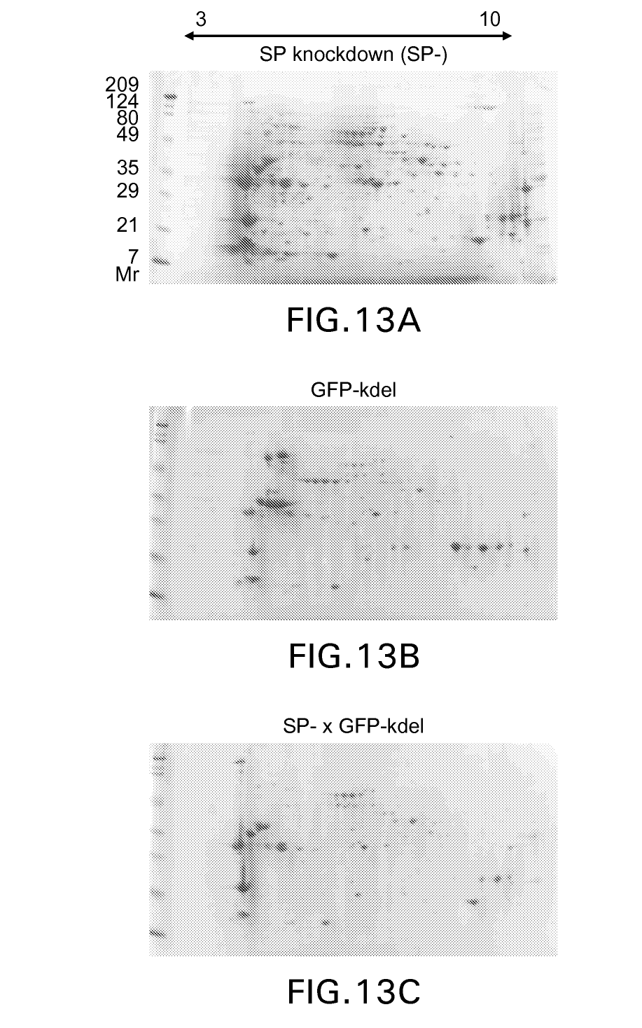

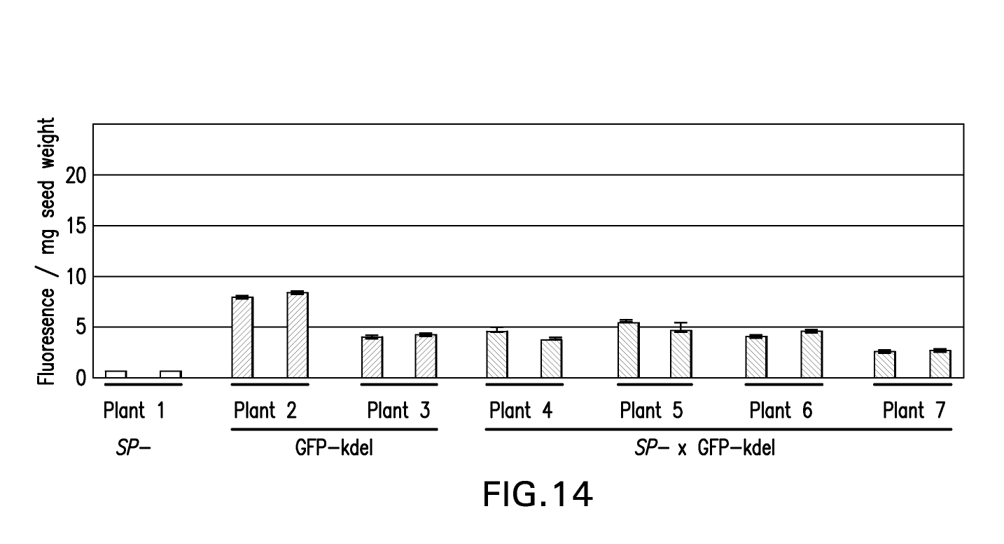

The GFP-kdel expression in the parental homozygous seeds results in accumulation of 1.6-2% GFP-kdel in the soybean assayed by spot volume comparison from 2D IEF/SDS-PAGE and by assays of seed lysates using a fluorometer assay with a standard curve control using commercially obtained GFP. These data are shown in comparison to data produced in the introgressed SP-plants are shown in FIG. 13 and FIG. 14.

The fluorescence light microscopy shows that GFP-kdel PBs are distributed throughout the cytoplasm. TEM-immunogold assays of the GFP-kdel PBs using a commercial MAb specific for GFP labeled 0.2-0.3 μm diameter ER-bounded PB-like structures as previously observed.

### Example 11

Production of βCS Soybean Lines

To further demonstrate the method of reducing the level of a seed storage protein, the transgenic soybean line (“βCS”), deficient in α/α subunit of β-conglycinin, was prepared by two different types of genetic knockdown. In one method, the plant was transformed with a “sense” construct having a β-conglycinin promoter driving the expression of FAD2 gene. This resulted in suppression of β-conglycinin.

In another example, a βCS line of soybean was made by transforming a soybean plant with an RNAi construct designed to suppress β-conglycinin. To prepare the RNAi construct, a sequence fragment from the β-conglycinin gene (genbankAB030495) (SEQ ID NO: 53) was placed adjacent to a 128 by fragment from the FAD2 gene (SEQ ID NO: 57). The entire 256 bp region is then placed in inverted repeats around an intron that was cloned using a pKannibal vector following the method described in Wesley et al., (2001) “Construct design for efficient, effective and high-throughput gene silencing in plants,” Plant J. 27, 581-590. The complete cassette sequence is shown in SEQ ID NO: 54.

Transformation using this sequence was found to suppress β-conglycinin levels in soybean seeds, resulting in the complete silencing of α/α′ β-conglycinin. A fraction of the increased production of glycinin was retained in the form of its precursor, proglycinin, and was sequestered in PBs.

### Example 12

### GFP-kdel Driven by the Glycinin Promoter in a βCS Soybean Line

Expressing a foreign protein as an extrinsic gene product should be relatively independent of intrinsic process of protein content rebalancing. The foreign protein selected, a GFP modified to include a KDEL ER retention sequence, is designed to accrete in the ER forming ER-derived protein bodies that are inert organelles, de novo created, and not normally found in soybean. Because the GFP-kdel bodies are stably accumulated through seed maturation (Schmidt and Herman 2008), the GFP-kdel can be quantified to measure its accumulation through seed maturation.

The GFP-kdel line was introgressed into a transgenic soybean line (“βCS”) deficient in the α/α subunit of β-conglycinin as a result of genetic knockdown. The resulting crosses were visually screened as mature dry seeds analyzed for GFP-kdel expression.

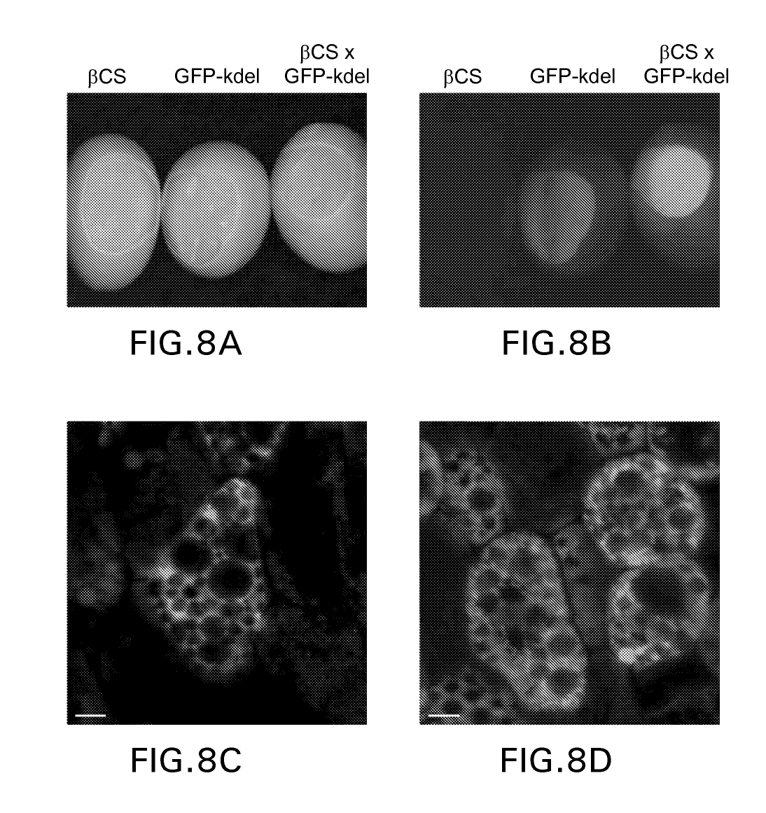

FIG. 8 shows white light (Panel A) and blue light (Panel B) imagery of GFP-kdel expression in soybeans. The soybeans were chipped and hydrated and used to image the subcellular distribution of GFP-kdel using a two-photon excitation Zeiss LSM 510 microscope with excitation at 488 nm using a 512 nm emission filter. FIG. 8 (bottom panel) shows GFP-kdel in the WT genetic background (Panel C), and in the βCS background (Panel D).

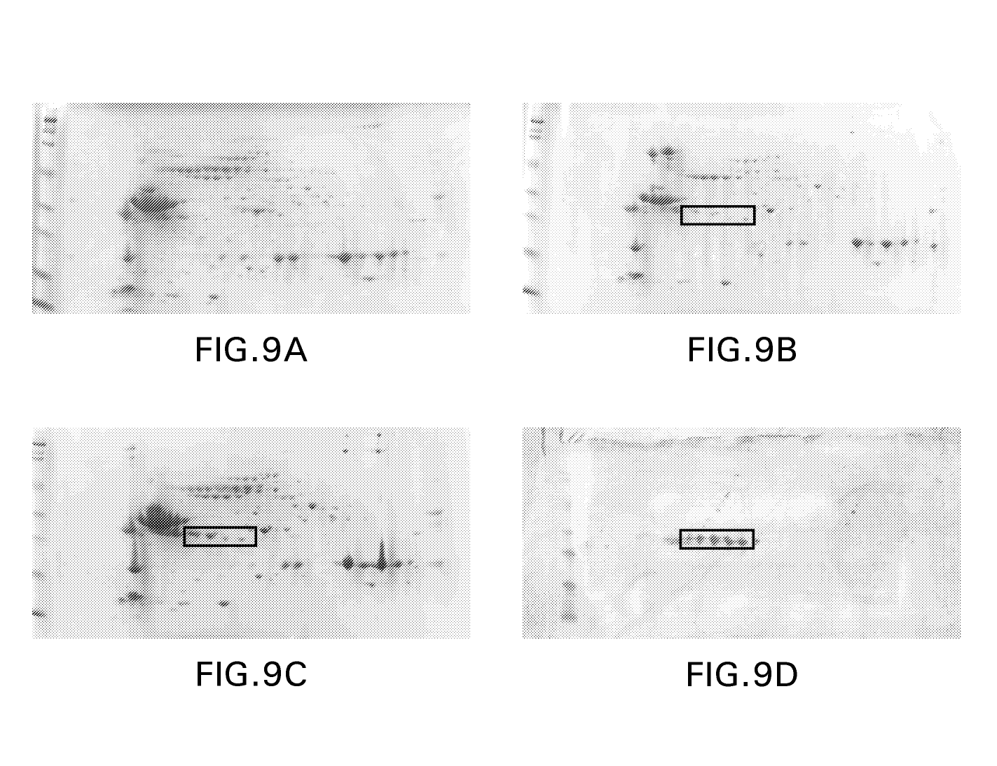

The remaining portion of the hydrated chip was used to produce 2D wide-range IEF/SDS-PAGE gels. Referring to FIG. 9, protein lysate from βCS seed is shown in Panel A, seed protein lysate from GFP-kdel expressed in a WT background is shown in Panel B, Protein lysate from βCS×GFP-kdel is shown in panel C, and an immuno-blot of replicate lysate gel (panel D) was used to identify which spots were GFP-kdel (boxed).

GFP-kdel accounts for approximately 2% of the seed proteome in the WT background and when crossed into βCS background, it increases to >7% of the seed proteome.

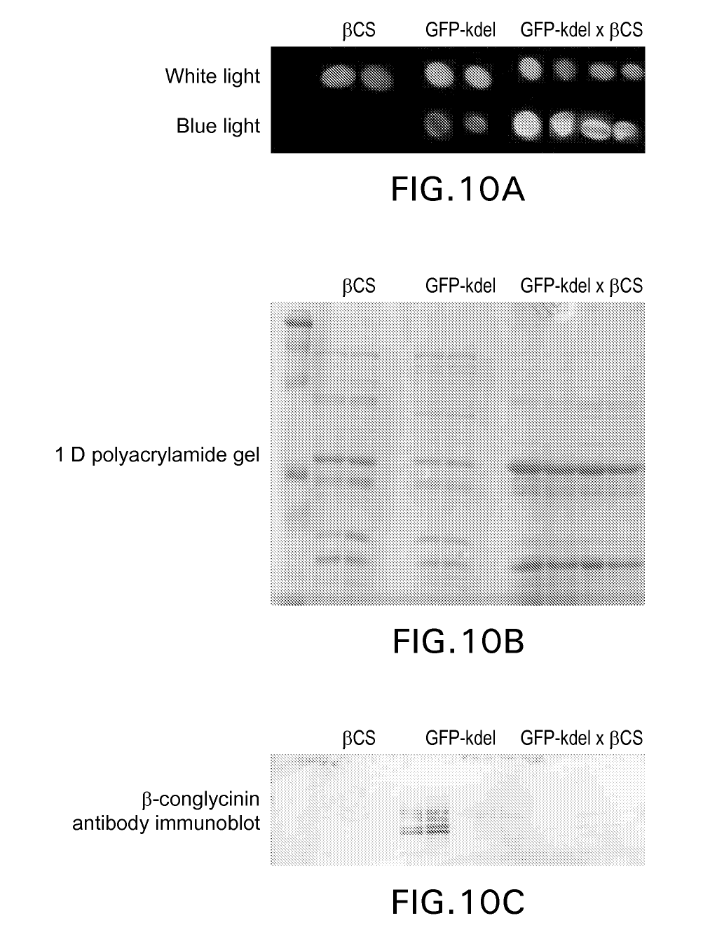

As shown in FIG. 10, (panel A), portions of the hydrated chips were visualized by fluorescence microscopy and the resulting images showed that the GFP-kdel is expressed at a higher level in a βCS background relative to a WT background.

Lysates of seed chips were then fractioned by 1D PAGE. FIG. 10 (Panel B) shows 1D gels of proteins from homozygous plants transformed expressing GFP-kdel in a WT background compared to a βCS background. FIG. 10 (Panel C) is an immunoblot using an antibody against β-conglycinin, confirming the lack of β-conglycinin protein in the βCS and βCS×GFP-kdel seed lysates.

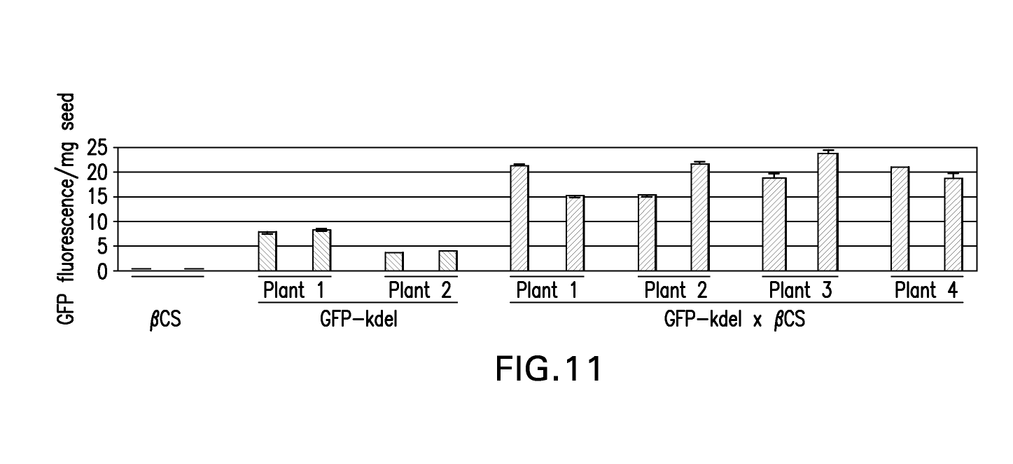

To obtain a further evaluation of the GFP-kdel abundance, seed lysates were prepared and assayed using a fluorometer with commercial GFP as a control standard. The results shown in FIG. 11 confirm both the visual impression of GFP-kdel fluorescence (FIG. 10, panel A) and the spot volume abundance (FIG. 9) that the GFP-kdel construct introgressed into a βCS plant results in about a 3.5 to 4.0 fold enhancement of GFP-kdel accumulation compared to a WT plant transformed with GFP-kdel.

The co-production and accumulation of proglycinin and GFP-kdel that accrete to form ER-derived protein bodies results in the formation of two distinct populations of ER-bounded protein bodies. De novo formed ER-derived protein bodies and ER sequestration of transgene products enhances the stability of otherwise post-translationally unstable proteins. The α/α subunit of β-conglycinin knockdown of soybeans produces proglycinin protein bodies and the introgression of GFP-kdel line also producing protein bodies results in the formation of a GFP-kdel protein bodies that are both more abundant in number and in size compared to the protein bodies in the GFP-kdel parent. Closely related proteins such as α and γ-zeins co-accrete, forming a single population of protein bodies. This is in contrast to the results of proglycinin and GFP-kdel, proteins which when expressed formed two distinct protein bodies with the GFP-kdel being favored for accretion into protein bodies. This indicates the accretion of proteins in the ER forming protein bodies is a protein-species-dependent process forming protein bodies with only one type of protein even if more than one protein-body-sequestered protein is synthesized. This is potentially advantageous for using protein body formation as a platform for biotechnology as it assures that the protein sequestered in the accretions will be relatively pure. The protein bodies can then be directly isolated from lysates, greatly simplifying the down-stream purification path.

### Example 13

### Processing of Heterologous Proteins in the βCS Soybean Line

Processing of the heterologous polynucleotide coding for glycinin was examined in WT and βCS soybean plants.

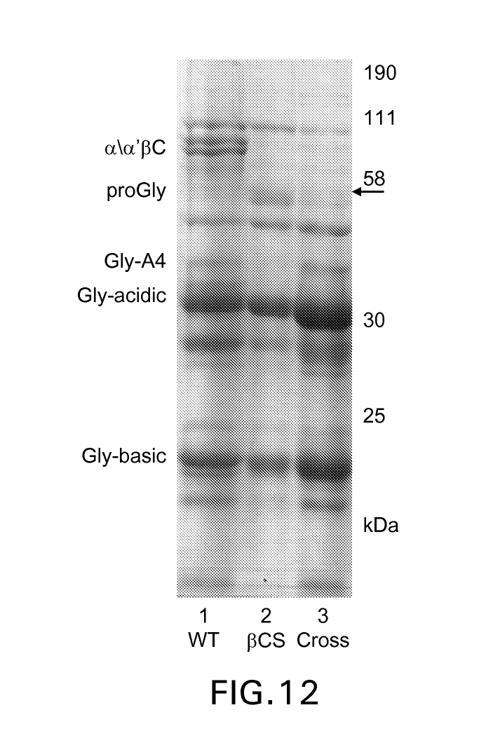

As shown in FIG. 12 (Lane 1), in WT (nontransgenic) soybean, no amount of pro-glycinin could be detected in seeds. Lane 2 shows that in the βCS plant, elevated levels of glycinin are stored in protein bodies as pro-glycinin. The percentage of pro-glycinin/glycinin is about ˜24%.

### Example 14

### Production of β-Glucosidase in Soybean Seeds

The constructs described herein can be used to produce enzymes such as β-glucosidase in soybean seeds. For example, the Aspergillus kawachii β-glucosidase sequence (SEQ ID NO: 35) may be inserted into a genetic construct having the soybean glycinin regulatory sequences, along with an ER signal and retention sequences. The construct may then be transformed into soybean tissue using biolistic bombardment, plants are regenerated and crossed, and stable homozygous plants are obtained. These plants are then introgressed into the βCS soybean lines. The production of protein of interest from these plants is then determined. Plants with a high expression level of β-glucosidase are chosen for scale-up production of the protein of interest.

### Example 15

### Production of Exocellobiohydrolase I in Soybean Seeds

The constructs described herein can be used to produce enzymes such as exocellobiohydrolase I in soybean seeds. For example, the modified Trichoderma reesei sequence exocellobiohydrolase I (Accession no. P26294), may be modified to include the Arabisopsis chitinase ER signal sequence and a KDEL retention signal sequence, as shown in SEQ ID NO: 22. The gene regulatory region from KTI (or the gene regulatory region of another protein that is found to compensate for the decreased protein in the soybean line having the genetic deficiency) may be used to drive expression of the protein. The construct may then be transformed into soybean tissue using biolistic bombardment, plants are then regenerated to the homozygous population for the expression of the enzyme. Homozygous plants expressing the enzyme may then be introgressed with homozygous SP-plants to form plants that are homozygous for the exocellobiohydrolase I enzyme and for the SP-trait. The production of exocellobiohydrolase I from these plants is then determined. Plants with a high expression level of exocellobiohydrolase I are preferably chosen for scale-up production of the protein.

### Example 16

### Processing of the Protein of Interest from Seeds

The protein can be utilized directly, unpurified from the soybean seed meal, or the protein of interest can be partially or substantially purified. The exact method will depend on the type of protein being produced, as well as the purity that will be needed. As an example, a transgenic protein of interest may be extracted from mature, dry soybeans by mixing the soybean seed grindate with 0.35 M NaCl in PBS at 75 g/L at room temperature for 2.5 hours. The extract may then be passed through several layers of cheesecloth, and centrifuged at 12,000 g for 1 hour at 4° C. The supernatant is then recovered and the NaCl concentration is adjusted to 0.4 M (pH 8.0). After a second centrifugation at 12,000 g for 10 minutes at 4° C., the supernatant may be collected and filtered through 0.45 μm nitrocellulose membrane. The filtrate may be loaded onto an SP SEPHAROSE™ column (Bio-Rad, Hercules, Calif.) which was previously equilibrated with 0.4 M NaCl in 50 mM sodium phosphate, pH 8.0 (binding buffer) at a flow rate of 5 ml/min. The column can be washed with the binding buffer until contaminants no longer elute. The protein of interest is then eluted by a linear gradient, followed by dialysis against PBS. The purified protein may then be analyzed, for example, by SDS-PAGE and/or immunoblot, and stored at −80° C., or, alternatively, lyophilized and stored at room temperature or below, until needed.

### Example 17

### Production of Other Enzymes in Soybean Seeds

A synthetic gene encoding a chosen industrial enzyme of interest may be inserted into the herein-described soybean seed-specific gene expression cassette that contains the 5′ and 3′ regulatory elements from either glycinin, KTI, P34, SBP, SMP or LE. The regulatory elements of KTI, P34, SBP, SMP, or LE will be used to drive the expression of the industrial enzymes, such as β-glucosidase, in an SP-background. The construct induces the industrial enzyme to participate in the protein rebalancing process resulting from the suppression of conglycinin and/or glycinin enhancing the synthesis and accumulation of the industrial enzyme.

The ORF preferably has a nucleotide sequence encoding the ER-targeting signal from the Arabidopsis chitinase basic gene fused 5′ and a nucleotide sequence encoding a carboxy-terminal KDEL ER retention sequence fused 3′ to the gene. The plasmid may also contain a hygromycin resistance marker for selection of transformants. Transformation and production of homozygous lines containing the gene of interest will be produced as described herein. The transgenic plants are then introgressed into either the SP-, βCS, or another seed storage protein deficient line. The amount of the transgenic protein in the seed will then be determined. Plants having a high level of expression of the industrial protein of interest will be used for scale-up production of the protein. By use of this method, an enhanced yield of the industrial protein of interest can be obtained.

### Example 18

### Production of a Human Antibody Fragment in Soybean Seeds

The methods described herein can be used to produce antibody fragments in soybean seeds. For example, an antibody fragment to be produced is chosen. The nucleic acid encoding the antibody fragment is then inserted into a vector construct having glycinin upstream and downstream regulatory elements, as described herein. The construct is then transformed to soybean seeds using electroporation. The transformed plants are then regenerated and crossed, and stable homozygous plants are obtained. These plants are then introgressed into a βCS line. The production of the antibody fragment is confirmed. Plants are grown for scale-up production of the protein of interest. The antibody fragments are then isolated and purified from the mature soybean seeds.

From the foregoing, it will be appreciated that, although specific embodiments of the invention have been described herein for the purpose of illustration, various modifications may be made without deviating from the spirit and scope of the invention. All references cited herein are incorporated by reference in their entireties.

TABLE 4
Nucleic Acid and Protein Sequences

| ORIGIN (GENE) | ORIGIN (SPECIES) | FUNCTION | SEQUENCE | SEQ ID NO: |
|---|---|---|---|---|
| Glycinin | Glycine max | promoter/ | caaaacaaattaataaaacacttacaacaccggatttttt | SEQ ID NO: 1 |
| | | upstream regulatory region | ttaattaaaatgtgccatttaggataaatagttaatattttt | |
| | | | aataattatttaaaaagccgtatctactaaaatgatttttat | |
| | | | ttggttgaaaatattaatatgtttaaatcaacacaatctat | |
| | | | caaaattaaactaaaaaaaaaataagtgtacgtggttaa | |
| | | | cattagtacagtaatataagaggaaaatgagaaattaa | |
| | | | gaaattgaaagcgagtctaatttttaaattatgaacctgc | |
| | | | atatataaaaggaaagaaagaatccaggaagaaaag | |
| | | | aaatgaaaccatgcatggtcccctcgtcatcacgagttt | |
| | | | ctgccatttgcaatagaaacactgaaacacctttctcttt | |
| | | | gtcacttaattgagatgccgaagccacctcacaccatg | |
| | | | aacttcatgaggtgtagcacccaaggcttccatagcca | |
| | | | tgcatactgaagaatgtctcaagctcagcaccctacttc | |
| | | | tgtgacgtgtccctcattcaccttcctctcttccctataaa | |
| | | | taaccacgcctcaggttctccgcttcacaactcaaacat | |
| | | | tctctccattggtccttaaacactcatcagtcatcaccgc | |
| Glycinin (Soybean Gy1 gene for | Glycine max | promoter/ | caaaacaaattaataaaacacttacaacaccggatttttt | SEQ ID NO: 2 |
| glycinin subunit | | upstream | ttaattaaaatgtgccatttaggataaatagttaatattttt | |
| G1; Accession number X15121.1) | | regulatory | aataattatttaaaaagccgtatctactaaaatgatttttat | |
| | | region | ttggttgaaaatattaatatgtttaaatcaacacaatctat | |
| | | | caaaattaaactaaaaaaaaaataagtgtacgtggttaa | |
| | | | cattagtacagtaatataagaggaaaatgagaaattaa | |
| | | | gaaattgaaagcgagtctaatttttaaattatgaacctgc | |
| | | | atatataaaaggaaagaaagaatccaggaagaaaag | |
| | | | aaatgaaaccatgcatggtcccctcgtcatcacgagttt | |
| | | | ctgccatttgcaatagaaacactgaaacacctttctcttt | |
| | | | gtcacttaattgagatgccgaagccacctcacaccatg | |
| | | | aacttcatgaggtgtagcacccaaggcttccatagcca | |
| | | | tgcatactgaagaatgtctcaagctcagcaccctacttc | |
| | | | tgtgacgttgtccctcattcaccttcctctcttccctataa | |
| | | | ataaccacgcctcaggttctccgcttcacaactcaaac | |
| | | | attctcctccattggtccttaaacactcatcagtcatcacc | |
| Conglycinin | Glycine max | promoter/ | gttttcaaatttgaattttaatgtgtgttgtaagtataaattt | SEQ ID NO: 3 |
| | | upstream | aaaataaaaataaaaacaattattatatcaaaatggcaa | |
| | | regulatory | aaacatttaatacgtattatttattaaaaaaatatgtaataa | |
| | | region | tatatttatattttaatatctattcttatgtattttttaaaaatct | |
| | | | attatatattgatcaactaaaatatttttatatctacacttatt | |
| | | | ttgcatttttatcaattttcttgcgttttttggcatatttaataa | |
| | | | tgactattctttaataatcaatcattattcttacatggtacat | |
| | | | attgttggaaccatatgaagtgttcattgcatttgactatg | |
| | | | tggatagtgttttgatccatgcccttcatttgccgctatta | |
| | | | attaatttggtaacagattcgttctaatcagttacttaatcc | |
| | | | ttcctcatcataattaatctggtagttcgaatgccataata | |
| | | | ttgattagttttttggaccataagaaaaagccaaggaac | |
| | | | aaaagaagacaaaacacaatgagagtatcctttgcata | |
| | | | gcaatgtctaagttcataaaattcaaacaaaaacgcaat | |
| | | | cacacacagtggacatcacttatccactagctgatcag | |
| | | | gatcgccgcgtcaagaaaaaaaaactggaccccaaa | |
| | | | agccatgcacaacaacacgtactcacaaaggcgtcaa | |
| | | | tcgagcagcccaaaacattcaccaactcaacccatcat | |
| | | | gagcccacacatttgttgtttctaacccaacctcaaact | |
| | | | cgtattctcttccgccacctcatttttgtttatttcaacacc | |
| | | | cgtcaaactgcatcccaccccgtggccaaatgttcatg | |
| | | | catgttaacaagacctatgactataaatatctgcaatctc | |
| | | | ggcccaagttttcatcatcaagaaccagttcaatatcct | |
| | | | agtacgccgtattaaagaatttaagatatact | |
| KTI | Glycine max | promoter/ | attgatactgataaaaaaatatcatgtgctttctggactg | SEQ ID NO: 4 |
| | | upstream | atgatgcagtatacttttgacattgcctttattttatttttca | |
| | | regulatory | gaaaagctttcttagttctgggttcttcattatttgtttccc | |
| | | region | atctccattgtgaattgaatcatttgcttcgtgtcacaaat | |
| | | | acatttagntaggtacatgcattggtcagattcacggttt | |
| | | | attatgtcatgacttaagttcatggtagtacattacctgcc | |
| | | | acgcatgcattatattggttagatttgataggcaaatttg | |
| | | | gttgtcaacaatataaatataaataatgtttttatattatga | |
| | | | aataacagtgatcaaaacaaacagttttatctttattaac | |
| | | | aagattttgtttttgtttgatgacgttttttaatgtttacgctt | |
| | | | cccccttcttttgaatttagaacactttatcatcataaaatc | |
| | | | aaatactaaaaaaattacatatttcataaataataacaca | |
| | | | aatatttttaaaaaatctgaaataataatgaacaatattac | |
| | | | atattatcacgaaaattcattaataaaaatattatataaat | |
| | | | aaaatgtaatagtagttatatgtaggttttttgtactgcac | |
| | | | gcataatatatacaaaaagattaaaatgaactattataaa | |
| | | | taataacactaaattaatggtgaatcatatcaaaataatg | |
| | | | aaaaagtaaataaaatttgtaattaacttctatatgtatta | |
| | | | cacacacaaataataaataatagtaaaaaaaattatgat | |
| | | | aaatatttaccatctcataaagatatttaaaataatgataa | |
| | | | aaatatagattattttttatgcaactagctagccaaaaag | |
| | | | agaacacgggtatatataaaaagagtacctttaaattct | |
| | | | actgtacttcctttattcctgacgtttttatatcaagtggac | |
| | | | atacgtgaagattttaattatcagtctaaatatttcattagc | |
| | | | acttaatacttttctgttttattcctatcctataagtagtccc | |
| | | | gattctcccaacattgcttattcacacaactaactaagaa | |
| | | | agtcttccatagccccccaaaa | |
| LE | Glycine max | promoter/ | aatgccatcgtatcgtgtcacaatggaatacagcaatg | SEQ ID NO: 5 |
| | | upstream | aacaaatgctatcctcttgagaaaagtgaaatgcagca | |
| | | regulatory | gcagcagcagactagagtgctacaaatgcttatcctctt | |
| | | region | gagaaaagtgaaatgcagcggcagcagacctgagtg | |
| | | | ctatatacaattagacacagggtctattaattgaaattgt | |
| | | | cttattattaaatatttcgttttatattaattttttaaattttaatt | |
| | | | aaatttatatatattatatttaagacagatatatttatttgtg | |
| | | | attataaatgtgtcactttttcttttagtccatgtattcttcta | |
| | | | ttttttcaatttaactttttatttttatttttaagtcactctgatc | |
| | | | aagaaaacattgttgacataaaactattaacataaaatta | |
| | | | tgttaacatgtgataacatcatattttactaatataacgtc | |
| | | | gcattttaacgtttttttaacaaatatcgactgtaagagta | |
| | | | aaaatgaaatgtttgaaaaggttaattgcatactaactat | |
| | | | tttttttcctataagtaatcttttttgggatcaattgtatatca | |
| | | | ttgagatacgatattaaatatgggtaccttttcacaaaac | |
| | | | ctacccttgttagtcaaaccacacataagagaggatgg | |
| | | | atttaaaccagtcagcaccgtaagtatatagtgaagaa | |
| | | | ggctgataacacactctattattgttagtacgtacgtattt | |
| | | | ccttttttgtttagtttttgaatttaattaattaaaatatatatg | |
| | | | ctaacaacattaaattttaaatttacgtctaattatatattgt | |
| | | | gatgtataataaattgtcaacctttaaaaattataaaaga | |
| | | | aatattaattttgataaacaacttttgaaaagtacccaata | |
| | | | atgctagtataaataggggcatgactccccatgcatca | |
| | | | cagtgcaatttagctgaagcaaagc | |
| P34 | Glycine max | promoter/ | atggataaaaaatctagcattctctcttttctcactagcat | SEQ ID NO: 6 |
| | | upstream | attaaattaacgatccagaaatatttataaatatttttttaat | |
| | | regulatory | gcttaatgactcaatacacggccagtcaaggtcaacct | |
| | | region | tggtcggataaacaaccctcataacatgacatacaatta | |
| | | | taatggaaaattctcatatagcacaattatgaaggcaaa | |
| | | | aacatggcacacaaaaggtacttgttttaactataaacg | |
| | | | attatgaatttttcagggaaaatagcatgttcgtcttgatt | |
| | | | ctcttgaagaactttttaggtaccttttactaagttggacg | |
| | | | tgattttgtctatgttcaggagaaaataaaggataaaatg | |
| | | | tcttttgtggacatgattttgattgtgatttcatgttaaggg | |
| | | | agtaaggatgatgtagttttatgtagagtttgtaggtcttg | |
| | | | gatctttttcattaatgaagtcatttgcttcttgaagatcaa | |
| | | | tgacaacaaaatgaagaagtaagaaaggtgattggag | |
| | | | acctcatttttaagaaaaaatgagtcaagaataagctta | |
| | | | ccaccataggaagtcatgaataagagcttgaaagtaa | |
| | | | gagaagatgagtggagggagagggagagagatgac | |
| | | | acaaaatttatgcctcaaataaggtctgaacattgaagt | |
| | | | ctaatttcttaaatgatcaaaattgaaaaaatacacacac | |
| | | | aagacctctatagtttaagtgtcattcaaaattggagaa | |
| | | | aaatttagatttctattcaaatttcacttgaatttgaatttat | |
| | | | agagtcaaattttgagtcaaaatttcattaattataatcag | |
| | | | tgaatttcaactatggtttagtctgctaatccaatatcaag | |
| | | | tccaaagttcttcactaagtgtgcttaggtgtcatgagg | |
| | | | catgtaaaatataaaggacatgtacaaagtatgaccat | |
| | | | atgatgtgacaatgaggtgtaacaagcaaatgctcacc | |
| | | | ttccctttaggctggtccaaaatttaattggattgagcttc | |
| | | | tcccaattcaattaaatttcttttttaacacacacatcaaat | |
| | | | agtgcactgaatgcacgtgaaattacaaaactatctca | |
| | | | aatacaaaaactagtctaggtgtcctaaaatacaaaga | |
| | | | ctgaaaaatcctatattatcagagtaccctccttacacta | |
| | | | tggagtcctaaatacaagactcaaaaataatgaaatcct | |
| | | | aatataatatatgtacaaaaataagtggattcatacttgg | |
| | | | tctattgatcgaaaatctaccttaaggctcatgagaatcc | |
| | | | taaggtcttctcctgcatctctgactcaatcttttaagtctc | |
| | | | caaccatgactttggta | |
| GBP | Glycine max | promoter/ | tggatatttaagtcttctataatatttcatttagagccaga | SEQ ID NO: 7 |
| | | upstream | agccaggttcaaaggaataggtaattcacatgaattcat | |
| | | regulatory | tctcttgtttctatacagctattatttttccatcttagtgttgc | |
| | | region | aggaaactacctcagttgttgtagatgtgcaaaacttgt | |
| | | | atggatatatatactgttcagtgttgggaaacccatgctt | |
| | | | tcttaattcacagagatacatttaaactttttttagaaactt | |
| | | | gcttagtatcttatcctgtttgttattcatttttggcagttgg | |
| | | | tcctaaagatactcctatgaatcttgtgctagagaagac | |
| | | | ttacgatgctaaaacaggacggggcatgcctgaacttt | |
| | | | aaggagacgttgccctgttccacttccaattaggtaact | |
| | | | gctatcgtgatgaacaaaaatttggtgagtttatcacctt | |
| | | | gccctttgccatgattcaattaaaagcgtgtttggacttt | |
| | | | ggaacctcattctaacaccaccctatgatgggttagac | |
| | | | gcaaaatctagactgggtagtgtttaacgtgtatctgtgt | |
| | | | gaacacagttacaaacgcattccttgtttaatgctaccat | |
| | | | gcctaggagttgaatcatttgtaactttaccaatttagtca | |
| | | | ttactactagcattcttttccctattcaagttgatgttagct | |
| | | | ccagttagtgatggtcatttcactctataaactttaattgtt | |
| | | | agatgagtggaagaggaacctgtttgattgttatggttc | |
| | | | tagttctagtgatttttattaattgggttcgaccatattagt | |
| | | | gtttgatttgagctatagatagttttttccccaaaagatca | |
| | | | gtcttctctcatgtcagattcatgggttggtactctttttat | |
| | | | ccagttccaacaaacttgctgttcgaactacgaagtca | |
| | | | gtcttacttattgggtaacatgtgggttttggtgtttaatg | |
| | | | gatctagaatactgtttgtagctaaacctatcttatcataa | |
| | | | agggcctaaaaagtaaaattggttattacatttggaaaa | |
| | | | aaagaaataatctaggcccactggcacactgagaaac | |
| | | | gttttcaatgaataatttaatagtttttttttataaaaaaatat | |
| | | | taataaaaaataatggagtttttaaaaatattacaacaat | |
| | | | ctgtttctctaaggttttttaatagttcagataattcatagct | |
| | | | tagagcaatacgacatggttaggaagcataaaaaaaa | |
| | | | tatacgacatggttaggaatttttttttagtatgtctgacat | |
| | | | aattttttaaatgttttggcttcatatgaatttaacagtgcg | |
| | | | tcatatgaacttacacactcattatattttttaaccttttaaa | |
| | | | tgattttaaaaaaaatatgacagatgcaatcttattttcac | |
| | | | tttttatactttcactactgcttcatatgacctaaagtcaga | |
| | | | gaaatattttaaaaagataaatacgataaagaatacgat | |
| | | | gagaaagaaacctcacacaatgaatagaccaaattag | |
| | | | acctatttattttccttagaaataaagaaaataattatttttt | |
| | | | attttttcacattacatttatatttttctatcactttctctattta | |
| | | | ggtattgattggcatatgagtgtacatgaatttttttttttaa | |
| | | | aaaaagcgtaaatattaattatattcatgcattatttgtttt | |
| | | | ctgtctttcattttctatttaatcttacgttatcaataatttatt | |
| | | | attaaattttatagttgatgatgaatatataagagatataa | |
| | | | ataaaaaaaataattaattttataataaaaattaaaaaata | |
| | | | attattttgagataaaaaattttaagagaacaattataaac | |
| | | | ggagagtattatatttagttttatgtaccgtgtacgtgtct | |
| | | | actaacatggtgtctctccatcattttcgtaggaaaaaac | |
| | | | attataggagtatgaaaaaagcaaaagttttgtctgttta | |
| | | | tggttttgtatatacccagctctacttggcagcaattacc | |
| | | | cgtcttgcttgctacttacgagacacgtacattaacactt | |
| | | | gtcctagctagtgcatgcaattgccaccccattcatcac | |
| | | | tcctcccttttccttctctttatatttatatatatatatatatat | |
| | | | atatatatatatatatatatatatatatatataaacaagcac | |
| | | | aatgcatcatctcaaagaaattaagagagattttttgttc | |
| | | | ctcactgaccaagcc | |
| SMP | Glycine max | promoter/ | gtttcacatgatccttcattctgtgtggcttaggagactc | SEQ ID NO: 8 |
| | | upstream | aacttcagagtccgtgatgatcaatgactcttaagttgtg | |
| | | regulatory | acttatggcttcatgtttaataaattacttcatagacaatg | |
| | | region | atgccctttcattattccatcccaaattaactaatgtttaa | |
| | | | gattttctttacacaaaactagaaaataaatattttaagag | |
| | | | aattaatatttagttgggggataattttaattattagaatat | |
| | | | tcttgttttctctcttaatttcaataaatatattagtaaattttt | |
| | | | taaaacaacattatgtatatatatatgtgaaaattagaata | |
| | | | aatattttgaatatttcaatatcaaccaatttaaatattttttt | |
| | | | aaaggttaaaattatagtcattttggaacatagacagta | |
| | | | atatggagctagagttatgttaaatacaggtcagaaaa | |
| | | | ataaaactcatgaaaatttgtaaaccaacgaaacttaca | |
| | | | ataagttcagcagtgatcttttgtctcatttttttacatttctt | |
| | | | tagtctctttattttttggttaaacgttgctttttagtttattat | |
| | | | gttttagtaatttttttttactttattacattaaattttaaaattt | |
| | | | ctattttgttttttttatgtttttgaaaattttattttgttaattttt | |
| | | | ttcataagaacactaatacttagaaaagaataaaaaaa | |
| | | | aaaaaggaaagttcgatggaattcaagctcatgaaattt | |
| | | | tatgttaaaaacatgagtaattcattaatattttaaaattta | |
| | | | aaataaaaaaaaaaaggaaagttcgatggaattcaag | |
| | | | ctcatgaaattttatgttaaaaacatgagtaattcattaat | |
| | | | attttaaaatttaaaataattaattgttttacttatattaaata | |
| | | | gttggtgaaaatttaaaaaaagatgcacgtttaaacaag | |
| | | | gttggaatcgttttgattttaatttcaccactcgagtggga | |
| | | | tgcacatttagacaaggatggaatcgtttgacttggattt | |
| | | | ggttactccttcccccaacacgctgccaccttctaggg | |
| | | | aaggtaagggaccgagtgaccgataacaacaccgat | |
| | | | ttccaaatatatatctcattcctaagctcacacacatctttt | |
| | | | acgttacatttcattattagatgctttcaatcgttaagaca | |
| | | | catgtcaccaccaaaagagcatctcataacaacgtgtc | |
| | | | acacctcccaagcacacgtgtcactcacaacacaacc | |
| | | | ctcacctatatataaattatcaaaccctccttccattcctc | |
| | | | cacatctcaatctcaatatctacacaaaagtgttccactt | |
| | | | gagtgaaaagtagtgtgttaagaactaaacaatttttca | |
| | | ER signal | mkimmmiklcffsmsliciapada | SEQ ID NO: 9 |
| | | sequence | | |
| | | ER signal | maashgnaifvlllctlflpslac | SEQ ID NO: 10 |
| | | sequence | | |
| | | ER signal | maarigifsvfvavllsisafssa | SEQ ID NO: 11 |
| | | sequence | | |
| | Arabidopsis | ER signal | mktnlflflifslllslssae | SEQ ID NO: 12 |
| | thaliana | sequence | | |
| | | from A. thaliana | | |
| | | basic | | |
| | | chitinase | | |
| Glycinin | Glycine max | Terminator | agccctttttgtatgtgctaccccacttttgtctttttggca | SEQ ID NO: 13 |
| | | (3′TED) | atagtgctagcaaccaataaataataataataataatga | |
| | | | ataagaaaacaaaggctttagcttgccttttgttcactgt | |
| | | | aaaataataatgtaagtactctctataatgagtcacgaa | |
| | | | acttttgcgggaataaaaggagaaattccaatgagtttt | |
| | | | ctgtcaaatcttcttttgtctctctctctctctctttttttttttt | |
| | | | ctttcttctgagcttcttgcaaaacaaaaggcaaacaat | |
| | | | aacgattggtccaatgatagttagcttgatcgatgatatc | |
| | | | tttaggaagtgttggcaggacaggacatgatgtagaa | |
| | | | gactaaaattgaaagtattgcagacccaatagttgaag | |
| | | | attaactttaagaatgaagacgtcttatcaggttcttcatg | |
| | | | acttgga | |
| Glycinin | Glycine max | Terminator | agccctttttgtatgtgctaccccacttttgtctttttggca | SEQ ID NO: 14 |
| (Soybean Gy1 | | (3′TED) | atagtgctagcaaccaataaataataataataataatga | |
| gene for | | | ataagaaaacaaaggctttagcttgccttttgttcactgt | |
| glycinin | | | aaaataataatgtaagtactctctataatgagtcacgaa | |
| subunit G1; | | | acttttgcgggaataaaaggagaaattccaatgagtttt | |
| accession | | | ctgtcaaatcttcttttgtctctctctctctcttttttttttct | |
| number | | | ttcttctgagcttcttgcaaaacaaaaggcaaacaataa | |
| X15121.1) | | | cgattggtccaatgatagttagcttgatcgatgatatcttt | |
| | | | aggaagtgttggcaggacaggacatgatgtagaaga | |
| | | | ctaaaattgaaagtattgcagacccaatagttgaagatt | |
| | | | aactttaagaatgaagacgtcttatcaggttcttcatgac | |
| | | | ttgga | |
| Beta- | Glycine max | Terminator | aataagtatgtagtactaaaatgtatgctgtaatagctca | SEQ ID NO: 15 |
| conglycinin | | (3′TED) | tagtgagcgaggaaagtatcgggctatttaactatgact | |
| | | | tgagctccatctatgaataaataaatcagcatatgatgct | |
| | | | tttgttttgtgtac | |
| beta- | Glycine max | Terminator | ataagtatgtagtactaaaatgtatgctgtaatagctcat | SEQ ID NO: 16 |
| conglycinin | | (3′TED) | agtgagcgaggaaagtatcgggctatttaactatgactt | |
| storage protein | | | gagctccatctatgaataaataaatcagcatatgatgctt | |
| (alpha′-bcsp) | | | ttgttttgtgtac | |
| gene; accession | | | | |
| number | | | | |
| M13759.1 | | | | |
| KTI | Glycine max | Terminator | gacacaagtgtgagagtactaaataaatgctttggttgt | SEQ ID NO: 17 |
| | | (3′TED) | acgaaatcattacactaaataaaataatcaaagcttatat | |
| | | | atgccttctaaggccgaatgcaaagaaattggttcttct | |
| | | | cgttatctttgccactttactagtacgtattaattactactt | |
| | | | aatcatcttgttacggctcattatatcc | |
| LE | Glycine max | Terminator | atgtgacagatcgaaggaagaaagtgtaataagacga | SEQ ID NO: 18 |
| | | (3′TED) | ctctcactactcgatcgctagtgattgtcattgttatatat | |
| | | | aataatgttatctttcacaacttatcgtaatgcattgtgaa | |
| | | | actataacacatttaatcctacttgtcatatgataacactc | |
| | | | tccccatttaaaactcttgtcaatttaaagatataagattc | |
| | | | tttaaatgattaaaaaaaatatattataaattcaatcactc | |
| | | | ctactaataaattattaattaatatttattgattaaaaaaat | |
| | | | acttatactaatttagtctgaatagaataattagattctag | |
| P34 | Glycine max | Terminator | gccgtaaaggttcaatacaacgagtgcttgttttcttag | SEQ ID NO: 19 |
| | | (3′TED) | ggacaagcattgtacttatgtatgattctgtgtaaccatg | |
| | | | agtcttccacgttgtactaatgtgaagggcaaaaataaa | |
| | | | acacagaacaagttcgtttttctcaaataatgtgaaggt | |
| | | | agaaaatggaaccatgcctcctctcttgcatgtgattta | |
| | | | aaatattagcagatggt | |
| GBP | Glycine max | Terminator | gaggtttagaacaatcaagaaaaggtgtgcatgtggct | SEQ ID NO: 20 |
| | | (3′TED) | gaagatcacggggaatgtattaagcttcagagactcttt | |
| | | | aaattaaattttctgtattttgtgttatatgttactagttcttta | |
| | | | aattagccagatggagtttatgtgtatctaaatgcaggg | |
| | | | atgctaatggaataaaatggccacttgtattgttagctat | |
| | | | ctcttatggtagcagaataagacgtaaactggttctttgc | |
| | | | tccaa | |
| SMP | Glycine max | Terminator | ttaaaacgtgatctatgatacaacaatattagtatatata | SEQ ID NO: 21 |
| | | (3′TED) | gacgcatgcagtttatatagtatatattgtcatgttgtatg | |
| | | | tttttacattttggtttgcttgtttacattctcttcaaaaaaaa | |
| | | | aaaaatgtgtagtacgtgtaaggttttgaagattggttct | |
| | | | aggctccgtgggaaccatttcaacaataaacattttgcg | |
| | | | cgttcttgtacacgtagtgatgagaagagatgccttatg | |
| | | | ggcagtatcatctaaaacttattttcatccatcatagaatt | |
| | | | tggatctattggactggactgaactgaactgaatgatcc | |
| | | | ttttttcttttttaatttcattcactaacaaatacataaaaca | |
| | | | ccagatattaacttagccagtatgaattttaactattttgtc | |
| | | | taatgctatgacttatcactgtctgtatcatctttaattctttt | |
| | | | ttcatattatttatattaaataa | |
| ER retention | Synthetic | ER retention | aaggatgaactttaatga | SEQ ID NO: 22 |
| tag (encodes | | signal | | |
| KDEL, | | sequence - | | |
| followed by 2 | | CDS | | |
| stop codons) | | | | |
| ER retention | Synthetic | ER retention - | kdel | SEQ ID NO: 23 |
| tag KDEL | | amino acid | | |
| | | sequence | | |
| | | (encoded by | | |
| | | above nucleic | | |
| | | acid | | |
| | | sequence) | | |
| ER retention | Synthetic | ER retention | aagcatgatgaactttaatga | SEQ ID NO: 24 |
| tag (encodes | | | | |
| KHDEL, | | | | |
| followed by 2 | | | | |
| stop codons) | | | | |
| ER retention | Synthetic | ER retention - | khdel | SEQ ID NO: 25 |
| tag KHDEL | | amino acid | | |
| | | sequence | | |
| | | (encoded by | | |
| | | above nucleic | | |
| | | acid | | |
| | | sequence) | | |
| ER retention | Synthetic | ER retention | hdel | SEQ ID NO: 26 |
| tag | | | | |
| ER retention | Synthetic | ER retention | keel | SEQ ID NO: 27 |
| tag | | | | |
| ER retention | Synthetic | ER retention | sekdel | SEQ ID NO: 28 |
| tag | | | | |
| ER retention | Synthetic | ER retention | sehdel | SEQ ID NO: 29 |
| tag | | | | |
| Ubiquitin 3 | Solanum | promoter/ | ccaaagcacatacttatcgatttaaatttcatcgaagag | SEQ ID NO: 30 |
| | tuberosum | upstream | attaatatcgaataatcatatacatactttaaatacataac | |
| | | regulatory | aaattttaaatacatatatctggtatataattaattttttaaa | |
| | | region | gtcatgaagtatgtatcaaatacacatatggaaaaaatt | |
| | | | aactattcataatttaaaaaatagaaaagatacatctagt | |
| | | | gaaattaggtgcatgtatcaaatacattaggaaaaggg | |
| | | | catatatcttgatctagataattaacgattttgatttatgtat | |
| | | | aatttccaaatgaaggtttatatctacttcagaaataaca | |
| | | | atatacttttatcagaacattcaacaaagcaacaaccaa | |
| | | | ctagagtgaaaaatacacattgttctctagacatacaaa | |
| | | | attgagaaaagaatctcaaaatttagagaaacaaatct | |
| | | | gaatttctagaagaaaaaaataattatgcactttgctatt | |
| | | | gctcgaaaaataaatgaaagaaattagacttttttaaaa | |
| | | | gatgttagactagatatactcaaaagctattaaaggagt | |
| | | | aatattcttcttacattaagtattttagttacagtcctgtaat | |
| | | | taaagacacattttagattgtatctaaacttaaatgtatct | |
| | | | agaatacatatatttgaatgcatcatatacatgtatccga | |
| | | | cacaccaattctcataaaaaacgtaatatcctaaactaa | |
| | | | tttatccttcaagtcaacttaagcccaatatacattttcatc | |
| | | | tctaaaggcccaagtggcacaaaatgtcaggcccaat | |
| | | | tacgaagaaaagggcttgtaaaaccctaataaagtgg | |
| | | | cactggcagagcttacactctcattccatcaacaaaga | |
| | | | aaccctaaaagccgcagcgccactgatttctctcctcc | |
| | | | aggcgaag | |
| Ubiquitin 3 | Solanum | terminator | ttgattttaatgtttagcaaatgtcttatcagttttctcttttt | SEQ ID NO: 31 |
| | tuberosum | | gtcgaacggtaatttagagttttttttgctatatggattttc | |
| | | | gtttttgatgtatgtgacaaccctcgggattgttgatttatt | |
| | | | tcaaaactaagagtttttgtcttattgttctcgtctattttgg | |
| | | | atatcaa | |
| Ubiquitin | Solanum | terminator | ttttaatgtttagcaaatgtcttatcagttttctctttttgtcg | SEQ ID NO: 32 |
| monomer/ | tuberosum | | aacggtaatttagagtttttttttgctatatggattttcgtttt | |
| ribosomal | | | tgatgtatgtgacaaccctcgggattgttgatttatttcaa | |
| protein; | | | aactaagagtttttgcttattgttcgtctattttggatatc | |
| (Genbank | | | aa | |
| Accession | | | | |
| number | | | | |
| Z11669.1) | | | | |
| Hygromycin | Escherichia coli | ORF | atgaaaaagcctgaactcaccgcgacgtctgtcgaga | SEQ ID NO: 33 |
| resistance gene | | | agtttctgatcgaaaagttcgacagcgtctccgacctg | |
| (hph) | | | atgcagctctcggagggcgaagaatctcgtgctttcag | |
| | | | cttcgatgtaggagggcgtggatatgtcctgcgggtaa | |
| | | | atagctgcgccgatggtttctacaaagatcgttatgttta | |
| | | | tcggcactttgcatcggccgcgctcccgattccggaa | |
| | | | gtgcttgacattggggcattcagcgagagcctgaccta | |
| | | | ttgcatctcccgccgtgcacagggtgtcacgttgcaag | |
| | | | acctgcctgaaaccgaactgcccgctgttctgcagcc | |
| | | | ggtcgcggaggccatggatgcgatcgctgcggccga | |
| | | | tcttagccagacgagcgggttcggcccattcggaccg | |
| | | | caaggaatcggtcaatacactacatggcgtgatttcata | |
| | | | tgcgcgattgctgatccccatgtgtatcactggcaaact | |
| | | | gtgatggacgacaccgtcagtgcgtccgtcgcgcag | |
| | | | gctctcgatgagctgatgctttgggccgaggactgccc | |
| | | | cgaagtccggcacctcgtgcacgcggatttcggctcc | |
| | | | aacaatgtcctgacggacaatggccgcataacagcg | |
| | | | gtcattgactggagcgaggcgatgttcggggattccc | |
| | | | aatacgaggtcgccaacatcttcttctggaggccgtgg | |
| | | | ttggcttgtatggagcagcagacgcgctacttcgagcg | |
| | | | gaggcatccggagcttgcaggatcgccgcggctccg | |
| | | | ggcgtatatgctccgcattggtcttgaccaactctatca | |
| | | | gagcttggttgacggcaatttcgatgatgcagcttggg | |
| | | | cgcagggtcgatgcgacgcaatcgtccgatccggag | |
| | | | ccgggactgtcgggcgtacacaaatcgcccgcagaa | |
| | | | gcgcggccgtctggaccgatggctgtgtagaagtact | |
| | | | cgccgatagtggaaaccgacgccccagcactcgtcc | |
| | | | gagggcaaaggaatag | |
| Green | Synthetic | | atgttcagtaaaggagaagaacttttcactggagttgtc | SEQ ID NO: 34 |
| Fluorescent | sequence - | | ccaattcttgttgaattagatggtgatgttaatgggcaca | |
| Protein (GFP) | (originally | | aattttctgtcagtggagagggtgaaggtgatgcaaca | |
| | derived from | | tacggaaaacttacccttaaatttatttgcactactggaa | |
| | Aequorea | | aactacctgttccatggccaacacttgtcactactttctct | |
| | Victoria | | tatggtgttcaatgcttttcaagatacccagatcatatga | |
| | | | agcggcacgacttcttcaagagcgccatgcctgaggg | |
| | | | atacgtgcaggagaggaccatctctttcaaggacgac | |
| | | | gggaactacaagacacgtgctgaagtcaagtttgagg | |
| | | | gagacaccctcgtcaacaggatcgagcttaagggaat | |
| | | | cgatttcaaggaggacggaaacatcctcggccacaa | |
| | | | gttggaatacaactacaactcccacaacgtatacatca | |
| | | | cggcagacaaacaaaagaatggaatcaaagctaactt | |
| | | | caaaattagacacaacattgaagatggaagcgttcaac | |
| | | | tagcagaccattatcaacaaaatactccaattggcgat | |
| | | | ggccctgtccttttaccagacaaccattacctgtccaca | |
| | | | caatctgccctttcgaaagatcccaacgaaaagagag | |
| | | | accacatggtccttcttgagtttgtaacagctgctggga | |
| | | | ttacacatggcatggatgaactatactga | |
| Beta glucosidase | Aspergillus kawachii | (original CDS | atgaggttcactttgattgaggcggtggctctcactgct | SEQ ID NO: 35 |
| | | sequence - AB003470 | gtctcgctggccagcgctgatgaattggcttactcccc | |
| | | | accgtattacccatccccttgggccaatggccagggc | |
| | | | gactgggcgcaggcataccagcgcgctgttgatattgt | |
| | | | ctcgcagatgacattggctgagaaggtcaatctgacca | |
| | | | caggaactggatgggaattggagctatgtgttggtcag | |
| | | | actggcggggttccccgattgggagttccgggaatgt | |
| | | | gtttacaggatagccctctgggcgttcgcgactccgac | |
| | | | tacaactctgctttcccttccggtatgaacgtggctgca | |
| | | | acctgggacaagaatctggcatacctccgcggcaag | |
| | | | gctatgggtcaggaatttagtgacaagggtgccgatat | |
| | | | ccaattgggtccagctgccggccctctcggtagaagt | |
| | | | cccgacggtggtcgtaactgggagggcttctcccccg | |
| | | | acccggccctaagtggtgtgctctttgcagagaccatc | |
| | | | aagggtatccaagatgctggtgtggtcgcgacggcta | |
| | | | agcactacattgcctacgagcaagagcatttccgtcag | |
| | | | gcgcctgaagcccaaggttatggatttaacatttccga | |
| | | | gagtggaagcgcgaacctcgacgataagactatgca | |
| | | | cgagctgtacctctggcccttcgcggatgccatccgtg | |
| | | | cgggtgctggcgctgtgatgtgctcctacaaccagatc | |
| | | | aacaacagctatggctgccagaacagctacactctga | |
| | | | acaagctgctcaaggccgagctgggtttccagggcttt | |
| | | | gtcatgagtgattgggcggctcaccatgctggtgtgag | |
| | | | tggtgctttggcaggattggatatgtctatgccaggaga | |
| | | | cgtcgactacgacagtggtacgtcttactggggtacaa | |
| | | | acctgaccgttagcgtgctcaacggaacggtgcccca | |
| | | | atggcgtgttgatgacatggctgtccgcatcatggccg | |
| | | | cctactacaaggtcggccgtgaccgtctgtggactcct | |
| | | | cccaacttcagctcatggaccagagatgaatacggct | |
| | | | acaagtactactatgtgtcggagggaccgtacgagaa | |
| | | | ggtcaaccactacgtgaacgtgcaacgcaaccacag | |
| | | | cgaactgatccgccgcattggagcggacagcacggt | |
| | | | gctcctcaagaacgacggcgctctgcctttgactggta | |
| | | | aggagcgcctggtcgcgcttatcggagaagatgcgg | |
| | | | gctccaacccttatggtgccaacggctgcagtgaccgt | |
| | | | ggatgcgacaatggaacattggcgatgggctgggga | |
| | | | agtggtactgccaacttcccatacctggtgacccccga | |
| | | | gcaggccatctcaaacgaggtgctcaagaacaagaat | |
| | | | ggtgtattcaccgccaccgataactgggctatcgatca | |
| | | | gattgaggcgcttgctaagaccgccagtgtctctcttgt | |
| | | | ctttgtcaacgccgactctggcgagggttacatcaatgt | |
| | | | cgacggaaacctgggtgaccgcaagaacctgaccct | |
| | | | gtggaggaacggcgataatgtgatcaaggctgctgct | |
| | | | agcaactgcaacaacaccattgttatcattcactctgtc | |
| | | | ggcccagtcttggttaacgaatggtacgacaaccca | |
| | | | atgttaccgctattctctggggtggtctgcccggtcagg | |
| | | | agtctggcaactctcttgccgacgtcctctatggccgtg | |
| | | | tcaaccccggtgccaagtcgccctttacctggggcaa | |
| | | | gactcgtgaggcctaccaagattacttggtcaccgagc | |
| | | | ccaacaacggcaatggagccccccaggaagacttcg | |
| | | | tcgagggcgtcttcattgactaccgcggattcgacaag | |
| | | | cgcaacgagaccccgatctacgagttcggctatggtct | |
| | | | gagctacaccactttcaactactcgaaccttgaggtgc | |
| | | | aggttctgagcgcccccgcgtacgagcctgcttcggg | |
| | | | tgagactgaggcagcgccaacttttggagaggttgga | |
| | | | aatgcgtcgaattacctctaccccgacggactgcaga | |
| | | | aaatcaccaagttcatctacccctggctcaacagtacc | |
| | | | gatctcgaggcatcttctggggatgctagctacggaca | |
| | | | ggactcctcggactatcttcccgagggagccaccgat | |
| | | | ggctctgcgcaaccgatcctgcctgctggtggcggtc | |
| | | | ctggcggcaaccctcgcctgtacgacgagctcatccg | |
| | | | cgtgtcggtgaccatcaagaacaccggcaaggttgct | |
| | | | ggtgatgaagttccccaactgtatgtttcccttggcggc | |
| | | | cccaacgagcccaagatcgtgctgcgtcaattcgagc | |
| | | | gcatcacgctgcagccgtcagaggagacgaagtgga | |
| | | | gcacgactctgacgcgccgtgaccttgcaaactggaa | |
| | | | tgttgagaagcaggactgggagattacgtcgtatccca | |
| | | | agatggtgtttgtcggaagctcctcgcggaagccgcc | |
| | | | gctccgggcgtctctgcctactgttcactaa | |
| Beta | Aspergillus | (full length | mrftlieavaltavslasadelaysppyypspwang | SEQ ID NO: 36 |
| glucosidase | kawachii | original | qgdwaqayqravdivsqmtlaekvnlttgtgwelel | |
| | (Accession No. BAA19913) | sequence | cvgqtggvprlgvpgmclqdsplgvrdsdynsafp | |
| | | (protein | sgmnvaatwdknlaylrgkamgqefsdkgadiql | |
| | | | gpaagplgrspdggrnwegfspdpalsgvlfaetik | |
| | | | giqdagvvatakhyiayeqehfrqapeaqgygfnis | |
| | | | esgsanlddktmhelylwpfadairagagavmcsy | |
| | | | nqinnsygcqnsytlnkllkaelgfqgfvmsdwaa | |
| | | | hhagvsgalagldmsmpgdvdydsgtsywgtnlt | |
| | | | vsvlngtvpqwrvddmavrimaayykvgrdrlwt | |
| | | | ppnfsswtrdeygykyyyvsegpyekvnhyvnv | |
| | | | qrnhselirrigadstvllkndgalpltgkerlvaliged | |
| | | | agsnpygangcsdrgcdngtlamgwgsgtanfpyl | |
| | | | vtpeqaisnevlknkngvftatdnwaidqiealakta | |
| | | | svslvfvnadsgegyinvdgnlgdrknltlwrngdn | |
| | | | vikaaasncnntiviihsvgpvlvnewydnpnvtai | |
| | | | lwgglpgqesgnsladvlygrvnpgakspftwgktr | |
| | | | eayqdylvtepnngngapqedfvegvfidyrgfdk | |
| | | | rnetpiyefgyglsyttfnysnlevqvlsapayepasg | |
| | | | eteaaptfgevgnasnylypdglqkitkfiypwlnst | |
| | | | dleassgdasygqdssdylpegatdgsaqpilpagg | |
| | | | gpggnprlydelirvsvtikntgkvagdevpqlyvsl | |
| | | | ggpnepkivlrqferitlqpseetkwsttltrrdlanw | |
| | | | nvekqdweitsypkmvfvgsssrkpplraslptvh | |
| Beta | Aspergillus | Sequence of | delaysppyypspwangqgdwaqayqravdivs | SEQ ID NO: 37 |
| glucosidase | kawachii | above with | qmtlaekvnlttgtgwelelcvgqtggvprlgvpgm | |
| | (partial | 19aa Signal | clqdsplgvrdsdynsafpsgmnvaatwdknlayl | |
| | sequence of | sequence | rgkamgqefsdkgadiqlgpaagplgrspdggrn | |
| | Accession No. | removed | wegfspdpalsgvlfaetikgiqdagvvatakhyia | |
| | BAA19913) | (protein | yeqehfrqapeaqgygfnisesgsanlddktmhely | |
| | | | lwpfadairagagavmcsynqinnsygcqnsytln | |
| | | | kllkaelgfqgfvmsdwaahhagvsgalagldms | |
| | | | mpgdvdydsgtsywgtnltvsvlngtvpqwrvdd | |
| | | | mavrimaayykvgrdrlwtppnfsswtrdeygyk | |
| | | | yyyvsegpyekvnhyvnvqrnhselirrigadstvll | |
| | | | kndgalpltgkerlvaligedagsnpygangcsdrg | |
| | | | cdngtlamgwgsgtanfpylvtpeqaisnevlknk | |
| | | | ngvftatdnwaidqiealaktasvslvfvnadsgegy | |
| | | | invdgnlgdrknltlwrngdnvikaaasncnntivii | |
| | | | hsvgpvlvnewydnpnvtailwgglpgqesgnsla | |
| | | | dvlygrvnpgakspftwgktreayqdylvtepnng | |
| | | | ngapqedfvegvfidyrgfdkrnetpiyefgyglsyt | |
| | | | tfnysnlevqvlsapayepasgeteaaptfgevgnas | |
| | | | nylypdglqkitkfiypwlnstdleassgdasygqds | |
| | | | sdylpegatdgsaqpilpagggpggnprlydelirvs | |
| | | | vtikntgkvagdevpqlyvslggpnepkivlrqferi | |
| | | | tlqpseetkwsttltrrdlanwnvekqdweitsypk | |
| | | | mvfvgsssrkpplraslptvh | |
| Beta | Aspergillus | (Substituted | MKTNLFLFLIFSLLLSLSSAEdelaysp | SEQ ID NO: 38 |
| glucosidase - | kawachii/with | signal | pyypspwangqgdwaqayqravdivsqmtlaek | |
| “GSF V1” | synthetic | sequence and | vnlttgtgwelelcvgqtggvprlgvpgmclqdspl | |
| | sequence | KDEL | gvrdsdynsafpsgmnvaatwdknlaylrgkamg | |
| | | sequences in | qefsdkgadiqlgpaagplgrspdggrnwegfspd | |
| | | CAPITAL | palsgvlfaetikgiqdagvvatakhyiayeqehfrq | |
| | | letters) | apeaqgygfnisesgsanlddktmhelylwpfadai | |
| | | | ragagavmcsynqinnsygcqnsytlnkllkaelgf | |
| | | | qgfvmsdwaahhagvsgalagldmsmpgdvdy | |
| | | | dsgtsywgtnltvsvlngtvpqwrvddmavrimaa | |
| | | | yykvgrdrlwtppnfsswtrdeygykyyyvsegp | |
| | | | yekvnhyvnvqrnhselirrigadstvllkndgalplt | |
| | | | gkerlvaligedagsnpygangcsdrgcdngtlam | |
| | | | gwgsgtanfpylvtpeqaisnevlknkngvftatdn | |
| | | | waidqiealaktasvslvfvnadsgegyinvdgnlg | |
| | | | drknltlwrngdnvikaaasncnntiviihsvgpvlv | |
| | | | newydnpnvtailwgglpgqesgnsladvlygrvn | |
| | | | pgakspftwgktreayqdylvtepnngngapqedf | |
| | | | vegvfidyrgfdkrnetpiyefgyglsyttfnysnlev | |
| | | | qvlsapayepasgeteaaptfgevgnasnylypdgl | |
| | | | qkitkfiypwlnstdleassgdasygqdssdylpega | |
| | | | tdgsaqpilpagggpggnprlydelirvsvtikntgk | |
| | | | vagdevpqlyvslggpnepkivlrqferitlqpseet | |
| | | | kwsttltrrdlanwnvekqdweitsypkmvfvgss | |
| | | | srkpplraslptvhKDEL | |
| Beta | Aspergillus | (Substituted | MK TNLFLFLIFS LLLSLSSAEd | SEQ ID NO: 39 |
| glucosidase | kawachii with | signal and | elaysppyyp spwangqgdw aqayqravdi | |
| “GSF V2” | synthetic | KDEL | vsqmtlDekvnlttgtgwel elcvgqtggv | |
| | sequence | sequences in | prlgvpgmcl qdsplgvrds dynsafpAgm | |
| | | CAPITAL | nvaatwdknlaylrgkamgq efsdkgadiq | |
| | | letters and | lgpaagplgr spdggrnweg fspdpalsgv | |
| | | the 13 amino | lfaetikgiqdagvvatakh yiayeqehfr | |
| | | acid | qapeaqgFgf nisesgsanl ddktmhelyl | |
| | | substitutions | wpfadairagagavmcsynq innsygcqns | |
| | | in | ytlnkllkae lgfqgfvmsd waahhagvsg | |
| | | underlined | alagldmsmp gdvdydsgts ywgtnltIsv | |
| | | CAPITAL | lngtvpqwry ddmavrimaa yykvgrdrlw | |
| | | letters) | tppnfsswtrdeygykyyyv segpyekvnQ | |
| | | | yvnvqrnhse lirrigadst vllkndgalp | |
| | | | ltgkerlvaligedagsnpy gangcsdrgc | |
| | | | dngtlamgwg sgtanfpylv tpeqaisnev | |
| | | | lkHkngvftatdnwaidqie alaktasysl | |
| | | | vfvnadsgeg yinvdgnlgd rRnltlwrng | |
| | | | dnvikaaasncnntivVihs vgpvlvnewy | |
| | | | dnpnvtailw gglpgqesgn sladvlygrv | |
| | | | npgakspftwgktreayqdy lvtepnngng | |
| | | | apqedfvegv fidyrgfdkr netpiyefgy | |
| | | | glsyttfnysnlevqvlsap ayepasgete | |
| | | | aaptfgevgn asDylypSgl qRitkfiypw | |
| | | | lnGtdleassgdasygqdss dylpegatdg | |
| | | | saqpilpagg gpggnprlyd elirvsvtik | |
| | | | ntgkvagdevpqlyvslggp nepkivlrqf | |
| | | | eritlqpsee tkwsttltrr dlanwnvekq | |
| | | | dweitsypkmvfvgsssrkL plraslptvh | |
| | | | KDEL | |
| Beta | Aspergillus | protein | mrftlieavaltavslasadelaysppyypspwang | SEQ ID NO: 40 |
| glucosidase | niger | | qgdwaqayqravdivsqmtldekvnlttgtgwelel | |
| sequence # 2 | | | cvgqtggvprlgvpgmclqdsplgvrdsdynsafp | |
| from U.S. Pat. | | | agmnvaatwdknlaylrgkamgqefsdkgadiql | |
| No. 7,223,902 | | | gpaagplgrspdggrnwegfspdpalsgvlfaetik | |
| (Acc. # | | | giqdagvvatakhyiayeqehfrqapeaqgfgfnis | |
| ABT13410.1) | | | esgsanlddktmhelylwpfadairagagavmcsy | |
| | | | nqinnsygcqnsytlnkllkaelgfqgfvmsdwaa | |
| | | | hhagvsgalagldmsmpgdvdydsgtsywgtnlti | |
| | | | svlngtvpqwrvddmavrimaayykvgrdrlwtp | |
| | | | pnfsswtrdeygykyyyvsegpyekvnqyvnvqr | |
| | | | nhselirrigadstvllkndgalpltgkerlvaligeda | |
| | | | gsnpygangcsdrgcdngtlamgwgsgtanfpyl | |
| | | | vtpeqaisnevlkhkngvftatdnwaidqiealakta | |
| | | | svslvfvnadsgegyinvdgnlgdrrnltlwrngdn | |
| | | | vikaaasncnntivvihsvgpvlvnewydnpnvtai | |
| | | | lwgglpgqesgnsladvlygrvnpgakspftwgktr | |
| | | | eayqdylvtepnngngapqedfvegvfidyrgfdk | |
| | | | rnetpiyefgyglsyttfnysnlevqvlsapayepasg | |
| | | | eteaaptfgevgnasdylypsgllritkfiypwlngtd | |
| | | | leassgdasygqdssdylpegatdgsaqpilpaggg | |
| | | | pggnprlydelirvsvtikntgkvagdevpqlyvslg | |
| | | | gpnepkivlrqferitlqpseetkwsttltrrdlanwn | |
| | | | vekqdweitsypkmvfvgsssrklplrasptvh | |
| Beta- | Aspergillus | CDS | atgaagctttccattttggaggcagcagctttgacagct | SEQ ID NO: 41 |
| glucosidase I | terreus | | gcctccgtagtcagcgcacaggacgatctcgcatact | |
| precursorXM_001212225 | NIH2624 | | ccccgccgtactacccttctccctgggccgatggcca | |
| | | | cggtgagtggtcgaacgcgtacaagcgcgctgtagat | |
| | | | atcgtctctcagatgacattgacggagaaggtcaatct | |
| | | | caccaccggtactggatgggagttggagaggtgtgtc | |
| | | | ggtcagacgggcagtgtccctagactgggaatcccaa | |
| | | | gcctctgtctgcaggatagccctctgggtattcgcatgt | |
| | | | cggactataactcggccttccctgcgggtattaacgttg | |
| | | | cggccacctgggacaagaagcttgcctaccaacgcg | |
| | | | gcaaggcaatgggcgaggaattcagtgacaagggta | |
| | | | ttgatgttcagttgggccctgctgccggtcctcttggca | |
| | | | ggtcccccgatggaggccgaaactgggagggcttct | |
| | | | ctcctgatcccgccctgactggtgtgttgttcgccgaga | |
| | | | cgatcaagggtatccaggacgccggagttattgctac | |
| | | | cgcgaagcactacattctcaacgaacaagagcatttcc | |
| | | | gccaggtcggcgaagcccagggctatggcttcaacat | |
| | | | caccgaaaccgtgagctcaaatgtggatgacaagacc | |
| | | | atgcacgagctgtatctctggcccttcgccgatgcggt | |
| | | | gcgcgcgggcgtgggcgctgtgatgtgctcctacaac | |
| | | | cagatcaacaacagctacggatgccaaaacagtttga | |
| | | | ccctgaacaagctcttgaaagccgaactcggatttcag | |
| | | | ggatttgtcatgagtgactggagtgctcaccacagcgg | |
| | | | tgttggcgccgccttggctggtttggacatgtccatgcc | |
| | | | gggagatatcagtttcgacagcggcacttccttctatgg | |
| | | | cacgaacctgactgttggcgtcctcaacggcaccattc | |
| | | | cccagtggcgtgtggatgacatggccgtccggatcat | |
| | | | ggctgcctactacaaggttggccgcgaccgtctctgg | |
| | | | actcctcccaatttcagctcgtggactcgcgatgaatat | |
| | | | ggcttcgcgcacttcttcccttccgaaggcgcttatgaa | |
| | | | cgtgtcaatgaattcgtcaacgtgcagcgtgaccatgc | |
| | | | ccaggtgatccgtcggattggcgcggatagtgtcgtg | |
| | | | ctcttgaagaacgacggtgcccttcccttgacgggcca | |
| | | | ggagaagactgttggcattctgggcgaagacgccgg | |
| | | | gtcgaatccgaagggagcaaatggttgcagtgaccgt | |
| | | | ggctgtgacaagggtactctggccatggcttggggta | |
| | | | gtggtactgccaacttcccttaccttgtgactcccgaac | |
| | | | aggccattcagaacgaggttctgaagggccgtggaa | |
| | | | atgtctttgccgtgacggacaactatgatacacagcag | |
| | | | attgccgccgttgcctctcaatccacggtttcattggtttt | |
| | | | cgtgaacgcagacgccggtgaaggtttccttaatgtgg | |
| | | | acggaaacatgggtgatcgcaagaacctcaccctctg | |
| | | | gcagaacggagaggaagtgatcaagactgtcacgga | |
| | | | gcactgcaacaacaccgttgttgtgatccattcggtgg | |
| | | | gacctgttctcatcgatgagtggtatgcgcaccccaat | |
| | | | gtcaccggcattctgtgggctggtctcccgggccagg | |
| | | | agtctggcaacgccattgcggacgtgctgtacggccg | |
| | | | cgtcaaccctggcggcaagaccccctttacctggggt | |
| | | | aagacgcgcgcgtcctacggcgactacctcctcaccg | |
| | | | agcccaacaacggcaacggtgctcctcaagacaactt | |
| | | | caacgagggcgtgtttattgactaccgtcgcttcgaca | |
| | | | agtacaatgagacgcccatctacgagttcggtcatggt | |
| | | | ctgagctacacgacgtttgagctgtctggcctccaggt | |
| | | | ccagcttatcaacggatccagctatgttcccactacgg | |
| | | | gtcagacgagcgccgcccaggcatttggtaaagtcga | |
| | | | ggacgcgtctagctacctgtaccctgagggactgaag | |
| | | | aggatttccaagttcatctatccctggctgaactctacc | |
| | | | gatcttaaagcgtctaccggcgatcctgaatacggaga | |
| | | | gcccaacttcgagtatattcctgaaggtgctaccgatg | |
| | | | gctctcctcagccccgtctgcctgccagcgggggtcc | |
| | | | tggcggcaaccccggtctctatgaggatctcttccagg | |
| | | | tttctgtgaccatcaccaacaccggcaaggttgctggt | |
| | | | gatgaggtgcctcagctgtatgtttcgctgggtggccc | |
| | | | caacgagccgaagcgggtgctgcgcaagttcgagcg | |
| | | | cctgcacatcgcccctggtcagcaaaaggtctggacg | |
| | | | actaccctgaaccgccgtgacctagccaactgggatg | |
| | | | tcgtggcccaggactggaagatcactccctatgctaag | |
| | | | accatctttgttggcacctcttcgcgcaagctgcctctc | |
| | | | gctggtcgcttgccacgggtgcagtaa | |
| Beta- | Aspergillus | Translation of | mklsileaaaltaasvvsaqddlaysppyypspwad | SEQ ID NO: 42 |
| glucosidaseXM_001212225 | terreus | above CDS | ghgewsnaykravdivsqmtltekvnlttgtgwele | |
| | NIH2624 | | rcvgqtgsvprlgipslclqdsplgirmsdynsafpa | |
| | | | ginvaatwdkklayqrgkamgeefsdkgidvqlgp | |
| | | | aagplgrspdggrnwegfspdpaltgvlfaetikgiq | |
| | | | dagviatakhyilneqehfrqvgeaqgygfnitetvs | |
| | | | snvddktmhelylwpfadavragvgavmcsynqi | |
| | | | nnsygcqnsltlnkllkaelgfqgfvmsdwsahhsg | |
| | | | vgaalagldmsmpgdisfdsgtsfygtnltvgvlngt | |
| | | | ipqwrvddmavrimaayykvgrdrlwtppnfssw | |
| | | | trdeygfahffpsegayervnefvnvqrdhaqvirri | |
| | | | gadsvvllkndgalpltgqektvgilgedagsnpkg | |
| | | | angcsdrgcdkgtlamawgsgtanfpylvtpeqai | |
| | | | qnevlkgrgnvfavtdnydtqqiaavasqstvslvfv | |
| | | | nadagegflnvdgnmgdrknltlwqngeeviktvt | |
| | | | ehcnntvvvihsvgpvlidewyahpnvtgilwagl | |
| | | | pgqesgnaiadvlygrvnpggktpftwgktrasyg | |
| | | | dylltepnngngapqdnfnegvfidyrrfdkynetpi | |
| | | | yefghglsyttfelsglqvglingssyvpttgqtsaaqa | |
| | | | fgkvedassylypeglkriskfiypwlnstdlkastg | |
| | | | dpeygepnfeyipegatdgspqprlpasggpggnp | |
| | | | glyedlfqvsvtitntgkvagdevpqlyvslggpnep | |
| | | | krvlrkferlhiapgqqkvwtttlnrrdlanwdvvaq | |
| | | | dwkitpyaktifvgtssrklplagrlprvq | |
| Exoglucanase 1 | Trichoderma | Full length | myrklavisaflataraqsactlqsethppltwqkcss | SEQ ID NO: 43 |
| (Accession No. | reesei | protein (aa 1-17 = | ggtctqqtgsvvidanwrwthatnsstncydgntws | |
| P62694) | | signal) | stlcpdnetcaknccldgaayastygvttsgnslsigf | |
| | | | vtqsaqknvgarlylmasdttyqeftllgnefsfdvd | |
| | | | vsqlpcglngalyfvsmdadggvskyptntagaky | |
| | | | gtgycdsqcprdlkfingqanvegwepssnnantgi | |
| | | | gghgsccsemdiweansisealtphpcttvgqeice | |
| | | | gdgcggtysdnryggtcdpdgcdwnpyrlgntsfy | |
| | | | gpgssftldttkkltvvtqfetsgainryyvqngvtfq | |
| | | | qpnaelgsysgnelnddyctaeeaefggssfsdkgg | |
| | | | ltqfkkatsggmvlvmslwddyyanmlwldstyp | |
| | | | tnetsstpgavrgscstssgvpaqvesqspnakvtfs | |
| | | | nikfgpigstgnpsggnppggnrgttttrrpatttgssp | |
| | | | gptqshygqcggigysgptvcasgttcqvlnpyysq | |
| | | | cl | |
| Modified | Trichoderma | Protein - | MKTNLFLFLIFSLLLSLSSAEqsactlqs | SEQ ID NO: 44 |
| exocellobiohydrolase | reesei | (above seq | ethppltwqkcssggtctqqtgsvvidanwrwthat | |
| I protein | | with ER | nsstncydgntwsstlcpdnetcaknccldgaayast | |
| | | signal | ygvttsgnslsigfvtqsaqknvgarlylmasdttyq | |
| | | replaced and | eftllgnefsfdvdvsqlpcglngalyfvsmdadgg | |
| | | added KDEL | vskyptntagakygtgycdsqcprdlkfingqanve | |
| | | in CAPITAL | gwepssnnantgigghgsccsemdiweansiseal | |
| | | letters) | tphpcttvgqeicegdgcggtysdnryggtcdpdgc | |
| | | | dwnpyrlgntsfygpgssftldttkkltvvtqfetsgai | |
| | | | nryyvqngvtfqqpnaelgsysgnelnddyctaeea | |
| | | | efggssfsdkggltqfkkatsggmvlvmslwddyy | |
| | | | anmlwldstyptnetsstpgavrgscstssgvpaqv | |
| | | | esqspnakvtfsnikfgpigstgnpsggnppggnrg | |
| | | | ttttrrpatttgsspgptqshygqcggigysgptvcas | |
| | | | gttcqvlnpyysqclKDEL | |
| Exoglucanase 2 | Trichoderma | Full length | mivgilttlatlatlaasvpleerqacssvwgqcggqn | SEQ ID NO: 45 |
| (Accession No. | reesei | protein (1-24 = | wsgptccasgstcvysndyysqclpgaassssstraa | |
| P07987) | | signal | sttsrvspttsrsssatpppgstttrvppvgsgtatysgn | |
| | | sequence) | pfvgvtpwanayyasevsslaipsltgamataaaav | |
| | | | akvpsfmwldtldktplmeqtladirtanknggnya | |
| | | | gqfvvydlpdrdcaalasngeysiadggvakykny | |
| | | | idtirqivveysdirtllviepdslanlvtnlgtpkcana | |
| | | | qsaylecinyavtqlnlpnvamyldaghagwlgw | |
| | | | panqdpaaqlfanvyknasspralrglatnvanyng | |
| | | | wnitsppsytqgnavyneklyihaigpllanhgwsn | |
| | | | affitdqgrsgkqptgqqqwgdwcnvigtgfgirps | |
| | | | antgdslldsfvwvkpggecdgtsdssaprfdshcal | |
| | | | pdalqpapqagawfqayfvqlltnanpsfl | |
| Modified | Trichoderma | Protein - | MKTNLFLFLIFSLLLSLSSAEqacssv | SEQ ID NO: 46 |
| Exoglucanase 2 | reesei | (above seq | wgqcggqnwsgptccasgstcvysndyysqclpg | |
| | | with ER | aassssstraasttsrvspttsrsssatpppgstttrvppv | |
| | | signal | gsgtatysgnpfvgvtpwanayyasevsslaipsltg | |
| | | replaced and | amataaaavakvpsfmwldtldktplmeqtladirt | |
| | | added KDEL | anknggnyagqfvvydlpdrdcaalasngeysiad | |
| | | in CAPITAL | ggvakyknyidtirqivveysdirtllviepdslanlvt | |
| | | letters) | nlgtpkcanaqsaylecinyavtqlnlpnvamylda | |
| | | | ghagwlgwpanqdpaaqlfanvyknasspralrgl | |
| | | | atnvanyngwnitsppsytqgnavyneklyihaig | |
| | | | pllanhgwsnaffitdqgrsgkqptgqqqwgdwc | |
| | | | nvigtgfgirpsantgdslldsfvwvkpggecdgtsd | |
| | | | ssaprfdshcalpdalqpapqagawfqayfvqlltna | |
| | | | npsflKDEL | |
| Endoglucanase | Acidothermus | Full length | mpralrrvpgsrvmlrvgvvvavlalvaalanlavp | SEQ ID NO: 47 |
| E1 (Accession | cellulolyticus | protein (1-41 = | rparaagggywhtsgreildannvpvriaginwfgf | |
| No. P54583) | | signal | etcnyvvhglwsrdyrsmldqikslgyntirlpysd | |
| | | sequence) | dilkpgtmpnsinfyqmnqdlqgltslqvmdkiva | |
| | | | yagqiglriildrhrpdcsgqsalwytssyseatwisd | |
| | | | lqalaqrykgnptvvgfdlhnephdpacwgcgdps | |
| | | | idwrlaaeragnavlsvnpnllifvegvqsyngdsy | |
| | | | wwggnlqgagqypvvlnvpnrlvysandyatsvy | |
| | | | pqtwfsdptfpnnmpgiwnknwgylfnqniapv | |
| | | | wlgefgttlqsttdqtwlktlvqylrptaqygadsfqw | |
| | | | tfwswnpdsgdtggilkddwqtvdtvkdgylapik | |
| | | | ssifdpvgasaspssqpspsyspspspspsasrtptpt | |
| | | | ptptasptptltptatptptasptpsptaasgarctasyq | |
| | | | vnsdwgngftvtvavtnsgsvatktwtvswtfggn | |
| | | | qtitnswnaavtqngqsvtarnmsynnviqpgqntt | |
| | | | fgfqasytgsnaaptvacaas | |
| Xylanase - | Aspergillus | CDS | atgaaggtcactgcggcttttgcaagtctcttgcttacg | SEQ ID NO: 48 |
| U39784 | niger | | gccttcgcggcccctgctccggagcctgttctggtgtc | |
| | | | gcgaagtgccggtatcaactacgtgcagaactacaac | |
| | | | ggcaaccttggtgacttcacctacgacgagagtaccg | |
| | | | ggacattttccatgtactgggaggatggagtcagttcc | |
| | | | gacttcgtcgttggtttgggctggaccactggctcctct | |
| | | | aaatctatcacctactctgcccaatacagcgcttctagc | |
| | | | tccagctcctacctggctgtctacggctgggtcaactct | |
| | | | cctcaggccgaatactacatcgtcgaggattacggtga | |
| | | | ttacaacccttgcagctcggccacgagccttggtaccg | |
| | | | tgtactctgatggaagcacctaccaagtctgcaccgac | |
| | | | actcgacgaacgcggccatctatcacaggaacaagc | |
| | | | acgttcacgcagtacttctccgttcgtgaaagtacacgc | |
| | | | acatccggaacagtgactatcgccaaccatttcaatttc | |
| | | | tgggcgcagcatgggttcggcaatagcaacttcaatta | |
| | | | tcaggtcatggcggtggaggcatggaacggtgtcgg | |
| | | | cagtgccagtgtcacgatctcctcttaa | |
| Xylanase - | Aspergillus | Protein | mkvtaafasllltafaapapepvlvsrsaginyvqny | SEQ ID NO: 49 |
| AAA99065.1 | niger | translation of | ngnlgdftydestgtfsmywedgvssdfvvglgwtt | |
| | | above | gssksitysaqysasssssylavygwvnspqaeyyi | |
| | | sequence | vedygdynpcssatslgtvysdgstyqvctdtrrtrps | |
| | | | itgtstftqyfsvrestrtsgtvtianhfnfwaqhgfgns | |
| | | | nfnyqvmaveawngvgsasvtiss | |
| Ligninase | Phanerochaete | Enzyme | mafkqlfaaislalslsaanaaaviekratcsngktvg | SEQ ID NO: 50 |
| 1508163A | chrysosporium | | dasccawfdvlddiqqnlfhggqcgaeahesirlvf | |
| | | | hdsiaispameaqgkfggggadgsimifddietafh | |
| | | | pnigldeivklqkpfvqkhgvtpgdfiafagavalsn | |
| | | | cpgapqmnfftgrapatqpapdglvpepfhtvdqii | |
| | | | nrvndagefdelelvwmlsahsvaavndvdptvq | |
| | | | glpfdstpgifdsqffvetqlrgtafpgsggnqgeves | |
| | | | plpgeiriqsdhtiardyrtacewqsfvnnqsklvdd | |
| | | | fqfiflaltqlgqdpnamtdcsdvipqskpipgnlpfs | |
| | | | ffpagktikdveqacaetpfptlttlpgpetsvqri | |
| Ligninase: | Trametes | Enzyme | vaxpdgvntatnaaxxqlfdggecgeevhesiarhx | SEQ ID NO: 51 |
| manganese | versicolor | | aigvsncpgapqigvsnxpgapqlardsrtaxewq | |
| peroxidase (EC | | | slliexselvpxpppalsnadveqaxaetpf | |
| 1.11.1.13) | | | | |
| Lignin | Phanerochaete | Enzyme | mafkqlfaaitvalsltaanaavvkekratcangktv | SEQ ID NO: 52 |
| peroxidase | chrysosporium | | gdasccawfdvlddiqanmfhggqcgaeahesirl | |
| (Accession No. | (a | | vfhdsiaispameakgkfggggadgsimifdtietaf | |
| P49012) | basidiomycete) | | hpnigldevvamqkpfvqkhgvtpgdfiafagava | |
| | | | lsncpgapqmnfftgrkpatqpapdglvpepfhtvd | |
| | | | qiiarvndagefdelelvwmlsahsvaavndvdpt | |
| | | | vqglpfdstpgifdsqffvetqfrgtlfpgsggnqgev | |
| | | | esgmageiriqtdhtlardsrtacewqsfvgnqsklv | |
| | | | ddfqfiflaltqlgqdpnamtdcsdviplskpipgng | |
| | | | pfsffppgkshsdieqacaetpfpslvtlpgpatsvar | |
| | | | ipphka | |
| RNAi sequence | Synthetic | RNAi | cctacagacttcaatctggtgatgccctgagagtcccct | SEQ ID NO: 53 |
| for BCS | | fragment- | caggaaccacatactatgtggtcaaccctgacaacaa | |
| (genbank | | suppresses | cgaaaatctcagattaataacactcgccatacccgttaa | |
| AB237643.1) | | conglycinin | caagcctggtagatttgag | |
| Chimeric | Synthetic | The complete | gcggccgcctcgagcctacagacttcaatctggtgat | SEQ ID NO: 54 |
| RNAi sequence | | RNAi cassette | gccctgagagtcccctcaggaaccacatactatgtggt | |
| | | to produce | caaccctgacaacaacgaaaatctcagattaataacac | |
| | | BCS | tcgccatacccgttaacaagcctggtagatttgaggat | |
| | | | atcgagctcgggctgtttctcttctcgtcacactcacaat | |
| | | | agggtggcctatgtatttagccttcaatgtctctggtaga | |
| | | | ccctatgatagttttgcaagccactaccacccttatgctc | |
| | | | ccatatattctaaccgtgaaagcttactagtctagtaccc | |
| | | | caattggtaaggaaataattattttcttttttccttttagtat | |
| | | | aaaatagttaagtgatgttaattagtatgattataataata | |
| | | | tagttgttataattgtgaaaaaataatttataaatatattgtt | |
| | | | tacataaacaacatagtaatgtaaaaaaatatgacaagt | |
| | | | gatgtgtaagacgaagaagataaaagttgagagtaag | |
| | | | tatattatttttaatgaatttgatcgaacatgtaagatgata | |
| | | | tactagcattaatatttgttttaatcataatagtaattctag | |
| | | | ctggtttgatgaattaaatatcaatgataaaatactatagt | |
| | | | aaaaataagaataaataaattaaaataatatttttttatgat | |
| | | | taatagtttattatataattaaatatctataccattactaaat | |
| | | | attttagtttaaaagttaataaatattttgttagaaattcca | |
| | | | atctgcttgtaatttatcaataaacaaaatattaaataaca | |
| | | | agctaaagtaacaaataatatcaaactaatagaaacag | |
| | | | taatctaatgtaacaaaacataatctaatgctaatataac | |
| | | | aaagcgtaaagctttcacggttagaatatatgggagca | |
| | | | taagggtggtagtggcttgcaaaactatcatagggtct | |
| | | | accagagacattgaaggctaaatacataggccaccct | |
| | | | attgtgagtgtgacgagaagagaaacagcccgagctc | |
| | | | gatatcctcaaatctaccaggcttgttaacgggtatggc | |
| | | | gagtgttattaatctgagattttcgttgttgtcagggttga | |
| | | | ccacatagtatgtggttcctgaggggactctcagggca | |
| | | | tcaccagattgaagtctgtaggctcgagtctagagcgg | |
| | | | ccgc | |
| Glycinin RNAi | synthetic | Storage | cattgacgagaccatttgcacaatgggacttcgccaca | SEQ ID NO: 55 |
| fragment | | protein RNAi | acataggccagacttcatcacctgacatcttcaaccctc | |
| (from glycinin | | sequence (to | aagctggtagcatcacaaccgctaccagcctcgacttc | |
| A1bB2 | | produce SP- | ccagccctctcgtggctcaaactcagtgcccagtttgg | |
| (genbank | | line) | atcactccgcaagaatgctatgttcgtgccacactaca | |
| AB030495) | | | acctgaacgcaaacagcataatatacgcattgaatgga | |
| | | | cgggcattggtacaagtggtgaattgcaatggtgaga | |
| | | | gagtgtttgatggagagctgcaagagggacaggtgtt | |
| | | | aactgtgccacaaaactttgcggtggctg | |
| Complete | synthetic | Complete | gcggccgcctcgagcattgacgagaccatttgcacaa | SEQ ID NO: 56 |
| glycinin RNAi | | cassette insert | tgggacttcgccacaacataggccagacttcatcacct | |
| insert | | used to | gacatcttcaaccctcaagctggtagcatcacaaccgc | |
| | | produce the | taccagcctcgacttcccagccctctcgtggctcaaac | |
| | | SP- | tcagtgcccagtttggatcactccgcaagaatgctatgt | |
| | | | tcgtgccacactacaacctgaacgcaaacagcataat | |
| | | | atacgcattgaatggacgggcattggtacaagtggtga | |
| | | | attgcaatggtgagagagtgtttgatggagagctgcaa | |
| | | | gagggacaggtgttaactgtgccacaaaactttgcggt | |
| | | | ggctggatatcgagctcgggctgtttctcttctcgtcac | |
| | | | actcacaatagggtggcctatgtatttagccttcaatgtc | |
| | | | tctggtagaccctatgatagttttgcaagccactaccac | |
| | | | ccttatgctcccatatattctaaccgtgaaagcttactag | |
| | | | tctagtaccccaattggtaaggaaataattattttctttttt | |
| | | | ccttttagtataaaatagttaagtgatgttaattagtatgat | |
| | | | tataataatatagttgttataattgtgaaaaaataatttata | |
| | | | aatatattgtttacataaacaacatagtaatgtaaaaaaa | |
| | | | tatgacaagtgatgtgtaagacgaagaagataaaagtt | |
| | | | gagagtaagtatattatttttaatgaatttgatcgaacatg | |
| | | | taagatgatatactagcattaatatttgttttaatcataata | |
| | | | gtaattctagctggtttgatgaattaaatatcaatgataaa | |
| | | | atactatagtaaaaataagaataaataaattaaaataata | |
| | | | tttttttatgattaatagtnattatataattaaatatctatacc | |
| | | | attactaaatattttagtttaaaagttaataaatattttgtta | |
| | | | gaaattccaatctgcttgtaatttatcaataaacaaaatat | |
| | | | taaataacaagctaaagtaacaaataatatcaaactaat | |
| | | | agaaacagtaatctaatgtaacaaaacataatctaatgc | |
| | | | taatataacaaagcgtaaagctttcacggttagaatatat | |
| | | | gggagcataagggtggtagtggcttgcaaaactatcat | |
| | | | agggtctaccagagacattgaaggctaaatacatagg | |
| | | | ccaccctattgtgagtgtgacgagaagagaaacagcc | |
| | | | cgagctcgatatccagccaccgcaaagttttgtggcac | |
| | | | agttaacacctgtccctcttgcagctctccatcaaacac | |
| | | | tctctcaccattgcaattcaccacttgtaccaatgcccgt | |
| | | | ccattcaatgcgtatattatgctgtttgcgttcaggttgta | |
| | | | gtgtggcacgaacatagcattcttgcggagtgatccaa | |
| | | | actgggcactgagtttgagccacgagagggctggga | |
| | | | agtcgaggctggtagcggttgtgatgctaccagcttga | |
| | | | gggttgaagatgtcaggtgatgaagtctggcctatgtt | |
| | | | gtggcgaagtcccattgtgcaaatggtctcgtcaatgct | |
| | | | cgagtctagagcggccgc | |
| FAD2 RNAi | synthetic | RNAi - | gggctgtttctcttctcgtcacactcacaatagggtggc | SEQ ID NO: 57 |
| (genbank | | suppresses | ctatgtatttagccttcaatgtctctggtagaccctatgat | |
| ab188250) | | FAD2 | agttttgcaagccactaccacccttatgctcccatatatt | |
| | | | ctaaccgtga | |

What is claimed:

1. A transgenic dicot plant comprising:
a. a deficiency of one or more seed storage proteins, wherein the deficiency results in an at least 50% reduction in endogenous seed storage protein compared to that of a wild type plant; and
b. a heterologous polynucleotide comprising a seed storage protein promoter, an open reading frame comprising an ER signal sequence, a desired protein coding sequence, and an ER retention signal; wherein the open reading frame is operably linked to the seed storage protein promoter; and
wherein the seed of the transgenic plant is capable of producing a heterologous protein at a level that is greater than 5% of the total dry weight of the seed.

2. The dicot plant of claim 1, wherein the heterologous polynucleotide further comprises a 5′ translational enhancer domain and/or a 3′ translational enhancer domain.

3. The dicot plant of claim 1 wherein the ER retention sequence induces accretion of the heterologous protein in the lumen of the ER or an ER-derived vesicle.

4. The dicot plant of claim 1, wherein said dicot is a member of the Fabaceae family, and optionally Fabales order, and optionally of soya genus.

5. The dicot plant of claim 4, wherein said dicot is a member of the Glycine genus.

6. The dicot plant of claim 5, wherein said dicot is a soybean.

7. The dicot plant of claim 1, wherein the promoter is derived from Kunitz trypsin inhibitor, soybean lectin, immunodominant soybean allergen P34 or Gly m Bd 30 k, glucose binding protein, seed maturation protein, glycinin, or conglycinin.

8. The dicot plant of claim 2, wherein the translational enhancer domain is derived from Kunitz trypsin inhibitor, Soybean lectin, immunodominant soybean allergen P34 or Gly m Bd 30 k, glucose binding protein, seed maturation protein, glycinin, or conglycinin.

9. The dicot plant of claim 1, wherein the storage protein deficiency is of one or more of Kunitz trypsin inhibitor, soybean lectin, immunodominant soybean allergen P34 or Gly m Bd 30 k, glucose binding protein, seed maturation protein, glycinin, or conglycinin.

10. The dicot plant of claim 9 wherein the dicot seed has more than an 75% deficiency of the seed's endogenous storage proteins.

11. The dicot plant of claim 1 further comprising a 5′ translational enhancer domain and a 3′ translational enhancer domain and wherein the promoter and the 3′ and the 5′ translational enhancer domains are derived from the same storage protein.

12. The dicot plant of claim 1 wherein the heterologous protein accumulates in a seed of the dicot to a level that is greater than about 2% or greater than about 4% or greater than about 5% of the seed's total dry weight.

13. A seed of the dicot plant of claim 1.

14. A transgenic protein obtained from the seed of claim 13.

15. The transgenic protein of claim 14, wherein the heterologous protein has been purified.

16. The dicot plant of claim 1, wherein the target protein coding sequence encodes an enzyme or fragment thereof.

17. The dicot plant of claim 16, wherein the enzyme is a cellulolytic enzyme.

18. The dicot plant of claim 17, wherein the cellulolytic enzyme is derived from a fungal source, a bacterial source, an animal source, or a plant source.

19. The dicot plant of claim 17, wherein the cellulolytic enzyme is a β-glucosidase, an Exoglucanase 1, an Exoglucanase II, an endoglucanase, a xylanase, a hemicellulase, a ligninase, a ligin peroxidase, or a manganese peroxidase.

20. A product comprising the protein of claim 14.

21. A commercially useful enzyme composition comprising the protein of claim 14.

22. The dicot plant of claim 1, wherein said deficiency of one or more seed storage proteins is due to the presence of an RNAi, an antisense, or a sense fragment of a nucleic acid encoding a seed storage protein.

23. A transgenic dicot plant comprising:
a. a deficiency of one or more endogenous plant storage proteins, wherein the deficiency results in an at least 50% reduction in the level of said endogenous plant storage protein compared to a wild type plant; and
b. a heterologous polynucleotide comprising a gene regulatory region of a compensating protein operably linked to an open reading frame encoding a sequence comprising an ER signal sequence, a desired protein coding sequence, and an ER retention signal;
wherein the seed of the transgenic dicot plant is capable of producing the heterologous protein at a level that is greater than 5% of the total dry weight of the seed.

24. A method of stably storing an enzyme prior to use, by storing said enzyme in a seed from a transgenic dicot plant comprising:
a. a deficiency of one or more plant storage proteins, wherein the deficiency results in an at least 50% reduction in endogenous seed protein; and
b. a heterologous polynucleotide comprising a seed storage protein promoter, an open reading frame comprising nucleic acid encoding an ER signal sequence, an enzyme of interest, and an ER retention signal; wherein the open reading frame is operably linked to the seed storage protein promoter; and
wherein the seed of the transgenic plant is capable of producing said enzyme at a level that is greater than 5% of the total dry weight of the seed; and
storing said enzyme in said seed of the transgenic dicot.

25. A method of producing an enhanced amount of a heterologous protein in a dicot plant, comprising:
a. stably transforming a plant cell with a polynucleotide comprising a seed storage protein promoter, an open reading frame comprising an ER signal sequence, a desired protein coding sequence, and an ER retention signal; wherein the open reading frame is operably linked to the seed storage protein promoter;
b. obtaining a homozygous plant line from said stably transformed plant cell;
c. introgressing said stably transformed plant line to a plant having a deficiency in an endogenous seed storage protein, wherein the deficiency results in an at least 50% reduction in said endogenous seed storage protein compared to that of a wild type plant;
d. growing the seeds of said introgressed transgenic plant; and
e. obtaining the heterologous protein from the seeds of the introgressed transgenic plant,
wherein said seed of the introgressed transgenic plant is capable of producing a heterologous protein at a level that is greater than 5% of the total dry weight of the seed.

26. The method of claim 25, wherein said deficiency in an endogenous seed storage protein is due to the presence of an RNAi, an antisense, or a sense fragment of a nucleic acid encoding a seed storage protein.

27. A method of producing an enhanced amount of a heterologous protein in a dicot plant, comprising:
a. stably transforming a plant cell with a polynucleotide comprising a seed storage protein promoter, an open reading frame comprising an ER signal sequence, a desired protein coding sequence, and an ER retention signal; wherein the open reading frame is operably linked to the seed storage protein promoter; wherein said polynucleotide further comprises an RNAi sequence that is capable of downregulation of an endogenous seed storage protein;
b. obtaining a homozygous plant line from said stably transformed plant cell;
c. growing the seeds of said homozygous plant line; and
d. obtaining the heterologous protein from the seeds of the homozygous plant.


SEQUENCE LISTING (57 sequences)

SEQ ID NO: 1 - DNA, 632 bp, Glycine max (soybean)
  promoter/upstream regulatory region of Glycinin
caaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgc

SEQ ID NO: 2 - DNA, 632 bp, Glycine max (soybean)
  Soybean Gy1 gene for glycinin subunit G1 (Accession No. X15121.1)
caaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgttgtccctcattcaccttcctctcttccctataaataaccacgcctcaggttctccgcttcacaactcaaacattctcctccattggtccttaaacactcatcagtcatcacc

SEQ ID NO: 3 - DNA, 962 bp, Glycine max (soybean)
  promoter/upstream regulatory region of Conglycinin
gttttcaaatttgaattttaatgtgtgttgtaagtataaatttaaaataaaaataaaaacaattattatatcaaaatggcaaaaacatttaatacgtattatttattaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgttcattgcatttgactatgtggatagtgttttgatccatgcccttcatttgccgctattaattaatttggtaacagattcgttctaatcagttacttaatccttcctcatcataattaatctggtagttcgaatgccataatattgattagttttttggaccataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggcgtcaatcgagcagcccaaaacattcaccaactcaacccatcatgagcccacacatttgttgtttctaacccaacctcaaactcgtattctcttccgccacctcatttttgtttatttcaacacccgtcaaactgcatcccaccccgtggccaaatgttcatgcatgttaacaagacctatgactataaatatctgcaatctcggcccaagttttcatcatcaagaaccagttcaatatcctagtacgccgtattaaagaatttaagatatact

SEQ ID NO: 4 - DNA, 1088 bp, Glycine max (soybean)
  promoter/upstream regulatory region of KTI
attgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattatgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggttttttgtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataaagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaaaa

SEQ ID NO: 5 - DNA, 966 bp, Glycine max (soybean)
  promoter/upstream regulatory region of LE
aatgccatcgtatcgtgtcacaatggaatacagcaatgaacaaatgctatcctcttgagaaaagtgaaatgcagcagcagcagcagactagagtgctacaaatgcttatcctcttgagaaaagtgaaatgcagcggcagcagacctgagtgctatatacaattagacacagggtctattaattgaaattgtcttattattaaatatttcgttttatattaattttttaaattttaattaaatttatatatattatatttaagacagatatatttatttgtgattataaatgtgtcactttttcttttagtccatgtattcttctattttttcaatttaactttttatttttatttttaagtcactctgatcaagaaaacattgttgacataaaactattaacataaaattatgttaacatgtgataacatcatattttactaatataacgtcgcattttaacgtttttttaacaaatatcgactgtaagagtaaaaatgaaatgtttgaaaaggttaattgcatactaactattttttttcctataagtaatcttttttgggatcaattgtatatcattgagatacgatattaaatatgggtaccttttcacaaaacctacccttgttagtcaaaccacacataagagaggatggatttaaaccagtcagcaccgtaagtatatagtgaagaaggctgataacacactctattattgttagtacgtacgtatttccttttttgtttagtttttgaatttaattaattaaaatatatatgctaacaacattaaattttaaatttacgtctaattatatattgtgatgtataataaattgtcaacctttaaaaattataaaagaaatattaattttgataaacaacttttgaaaagtacccaataatgctagtataaataggggcatgactccccatgcatcacagtgcaatttagctgaagcaaagc

SEQ ID NO: 6 - DNA, 1369 bp, Glycine max (soybean)
  promoter/upstream regulatory region of P34
atggataaaaaatctagcattctctcttttctcactagcatattaaattaacgatccagaaatatttataaatatttttttaatgcttaatgactcaatacacggccagtcaaggtcaaccttggtcggataaacaaccctcataacatgacatacaattataatggaaaattctcatatagcacaattatgaaggcaaaaacatggcacacaaaaggtacttgttttaactataaacgattatgaatttttcagggaaaatagcatgttcgtcttgattctcttgaagaactttttaggtaccttttactaagttggacgtgattttgtctatgttcaggagaaaataaaggataaaatgtcttttgtggacatgattttgattgtgatttcatgttaagggagtaaggatgatgtagttttatgtagagtttgtaggtcttggatctttttcattaatgaagtcatttgcttcttgaagatcaatgacaacaaaatgaagaagtaagaaaggtgattggagacctcatttttaagaaaaaatgagtcaagaataagcttaccaccataggaagtcatgaataagagcttgaaagtaagagaagatgagtggagggagagggagagagatgacacaaaatttatgcctcaaataaggtctgaacattgaagtctaatttcttaaatgatcaaaattgaaaaaatacacacacaagacctctatagtttaagtgtcattcaaaattggagaaaaatttagatttctattcaaatttcacttgaatttgaatttatagagtcaaattttgagtcaaaatttcattaattataatcagtgaatttcaactatggtttagtctgctaatccaatatcaagtccaaagttcttcactaagtgtgcttaggtgtcatgaggcatgtaaaatataaaggacatgtacaaagtatgaccatatgatgtgacaatgaggtgtaacaagcaaatgctcaccttccctttaggctggtccaaaatttaattggattgagcttctcccaattcaattaaatttcttttttaacacacacatcaaatagtgcactgaatgcacgtgaaattacaaaactatctcaaatacaaaaactagtctaggtgtcctaaaatacaaagactgaaaaatcctatattatcagagtaccctccttacactatggagtcctaaatacaagactcaaaaataatgaaatcctaatataatatatgtacaaaaataagtggattcatacttggtctattgatcgaaaatctaccttaaggctcatgagaatcctaaggtcttctcctgcatctctgactcaatcttttaagtctccaaccatgactttggta

SEQ ID NO: 7 - DNA, 2262 bp, Glycine max (soybean)
  promoter/upstream regulatory region of GBP
tggatatttaagtcttctataatatttcatttagagccagaagccaggttcaaaggaataggtaattcacatgaattcattctcttgtttctatacagctattatttttccatcttagtgttgcaggaaactacctcagttgttgtagatgtgcaaaacttgtatggatatatatactgttcagtgttgggaaacccatgctttcttaattcacagagatacatttaaactttttttagaaacttgcttagtatcttatcctgtttgttattcatttttggcagttggtcctaaagatactcctatgaatcttgtgctagagaagacttacgatgctaaaacaggacggggcatgcctgaactttaaggagacgttgccctgttccacttccaattaggtaactgctatcgtgatgaacaaaaatttggtgagtttatcaccttgccctttgccatgattcaattaaaagcgtgtttggactttggaacctcattctaacaccaccctatgatgggttagacgcaaaatctagactgggtagtgtttaacgtgtatctgtgtgaacacagttacaaacgcattccttgtttaatgctaccatgcctaggagttgaatcatttgtaactttaccaatttagtcattactactagcattcttttccctattcaagttgatgttagctccagttagtgatggtcatttcactctataaactttaattgttagatgagtggaagaggaacctgtttgattgttatggttctagttctagtgatttttattaattgggttcgaccatattagtgtttgatttgagctatagatagttttttccccaaaagatcagtcttctctcatgtcagattcatgggttggtactctttttatccagttccaacaaacttgctgttcgaactacgaagtcagtcttacttattgggtaacatgtgggttttggtgtttaatggatctagaatactgtttgtagctaaacctatcttatcataaagggcctaaaaagtaaaattggttattacatttggaaaaaaagaaataatctaggcccactggcacactgagaaacgttttcaatgaataatttaatagtttttttttataaaaaaatattaataaaaaataatggagtttttaaaaatattacaacaatctgtttctctaaggttttttaatagttcagataattcatagcttagagcaatacgacatggttaggaagcataaaaaaaatatacgacatggttaggaatttttttttagtatgtctgacataattttttaaatgttttggcttcatatgaatttaacagtgcgtcatatgaacttacacactcattatattttttaaccttttaaatgattttaaaaaaaatatgacagatgcaatcttattttcactttttatactttcactactgcttcatatgacctaaagtcagagaaatattttaaaaagataaatacgataaagaatacgatgagaaagaaacctcacacaatgaatagaccaaattagacctatttattttccttagaaataaagaaaataattattttttattttttcacattacatttatatttttctatcactttctctatttaggtattgattggcatatgagtgtacatgaatttttttttttaaaaaaagcgtaaatattaattatattcatgcattatttgttttctgtctttcattttctatttaatcttacgttatcaataatttattattaaattttatagttgatgatgaatatataagagatataaataaaaaaaataattaattttataataaaaattaaaaaataattattttgagataaaaaattttaagagaacaattataaacggagagtattatatttagttttatgtaccgtgtacgtgtctactaacatggtgtctctccatcattttcgtaggaaaaaacattataggagtatgaaaaaagcaaaagttttgtctgtttatggttttgtatatacccagctctacttggcagcaattacccgtcttgcttgctacttacgagacacgtacattaacacttgtcctagctagtgcatgcaattgccaccccattcatcactcctcccttttccttctctttatatttatatatatatatatatatatatatatatatatatatatatatatatatataaacaagcacaatgcatcatctcaaagaaattaagagagttttttttgttcctcactgaccaagcc

SEQ ID NO: 8 - DNA, 1387 bp, Glycine max (soybean)
  promoter/upstream regulatory region of SMP
gtttcacatgatccttcattctgtgtggcttaggagactcaacttcagagtccgtgatgatcaatgactcttaagttgtgacttatggcttcatgtttaataaattacttcatagacaatgatgccctttcattattccatcccaaattaactaatgtttaagattttctttacacaaaactagaaaataaatattttaagagaattaatatttagttgggggataattttaattattagaatattcttgttttctctcttaatttcaataaatatattagtaaattttttaaaacaacattatgtatatatatatgtgaaaattagaataaatattttgaatatttcaatatcaaccaatttaaatatttttttaaaggttaaaattatagtcattttggaacatagacagtaatatggagctagagttatgttaaatacaggtcagaaaaataaaactcatgaaaatttgtaaaccaacgaaacttacaataagttcagcagtgatcttttgtctcatttttttacatttctttagtctctttattttttggttaaacgttgctttttagtttattatgttttagtaatttttttttactttattacattaaattttaaaatttctattttgttttttttatgtttttgaaaattttattttgttaatttttttcataagaacactaatacttagaaaagaataaaaaaaaaaaaggaaagttcgatggaattcaagctcatgaaattttatgttaaaaacatgagtaattcattaatattttaaaatttaaaataaaaaaaaaaaggaaagttcgatggaattcaagctcatgaaattttatgttaaaaacatgagtaattcattaatattttaaaatttaaaataattaattgttttacttatattaaatagttggtgaaaatttaaaaaaagatgcacgtttaaacaaggttggaatcgttttgattttaatttcaccactcgagtgggatgcacatttagacaaggatggaatcgtttgacttggatttggttactccttcccccaacacgctgccaccttctagggaaggtaagggaccgagtgaccgataacaacaccgatttccaaatatatatctcattcctaagctcacacacatcttttacgttacatttcattattagatgctttcaatcgttaagacacatgtcaccaccaaaagagcatctcataacaacgtgtcacacctcccaagcacacgtgtcactcacaacacaaccctcacctatatataaattatcaaaccctccttccattcctccacatctcaatctcaatatctacacaaaagtgttccacttgagtgaaaagtagtgtgttaagaactaaacaatttttca

SEQ ID NO: 9 - protein, 24 aa, Arabidopsis thaliana
  ER signal sequence of alpha carbonic anhydrase I gene (NP_850685)
MKIMMMIKLCFFSMSLICIAPADA

SEQ ID NO: 10 - protein, 24 aa, Oryza sativa Japonica
  ER signal sequence from an unnamed protein (BAG87020)
MAASHGNAIFVLLLCTLFLPSLAC

SEQ ID NO: 11 - protein, 24 aa, Arabidopsis thaliana
  ER signal sequence of ribophorin I (AT2G01720)
MAARIGIFSVFVAVLLSISAFSSA

SEQ ID NO: 12 - protein, 21 aa, Arabidopsis thaliana
  ER signal sequence from A. thaliana basic chitinase
MKTNLFLFLIFSLLLSLSSAE

SEQ ID NO: 13 - DNA, 447 bp, Glycine max (soybean)
  Terminator (3-prime TED) of Glycinin
agccctttttgtatgtgctaccccacttttgtctttttggcaatagtgctagcaaccaataaataataataataataatgaataagaaaacaaaggctttagcttgccttttgttcactgtaaaataataatgtaagtactctctataatgagtcacgaaacttttgcgggaataaaaggagaaattccaatgagttttctgtcaaatcttcttttgtctctctctctctctcttttttttttttctttcttctgagcttcttgcaaaacaaaaggcaaacaataacgattggtccaatgatagttagcttgatcgatgatatctttaggaagtgttggcaggacaggacatgatgtagaagactaaaattgaaagtattgcagacccaatagttgaagattaactttaagaatgaagacgtcttatcaggttcttcatgacttgga

SEQ ID NO: 14 - DNA, 445 bp, Glycine max (soybean)
  Terminator (3-prime TED) of Soybean Gy1 gene for glycinin subunit G1 (Accession No. X15121.1)
agccctttttgtatgtgctaccccacttttgtctttttggcaatagtgctagcaaccaataaataataataataataatgaataagaaaacaaaggctttagcttgccttttgttcactgtaaaataataatgtaagtactctctataatgagtcacgaaacttttgcgggaataaaaggagaaattccaatgagttttctgtcaaatcttcttttgtctctctctctctctcttttttttttctttcttctgagcttcttgcaaaacaaaaggcaaacaataacgattggtccaatgatagttagcttgatcgatgatatctttaggaagtgttggcaggacaggacatgatgtagaagactaaaattgaaagtattgcagacccaatagttgaagattaactttaagaatgaagacgtcttatcaggttcttcatgacttgga

SEQ ID NO: 15 - DNA, 133 bp, Glycine max (soybean)
  Terminator (3-prime TED) of Beta-conglycinin
aataagtatgtagtactaaaatgtatgctgtaatagctcatagtgagcgaggaaagtatcgggctatttaactatgacttgagctccatctatgaataaataaatcagcatatgatgcttttgttttgtgtac

SEQ ID NO: 16 - DNA, 132 bp, Glycine max (soybean)
  Terminator (3-prime TED) of Beta-conglycinin storage protein (alpha-prime-bcsp) gene (Accession No. M13759.1)
ataagtatgtagtactaaaatgtatgctgtaatagctcatagtgagcgaggaaagtatcgggctatttaactatgacttgagctccatctatgaataaataaatcagcatatgatgcttttgttttgtgtac

SEQ ID NO: 17 - DNA, 188 bp, Glycine max (soybean)
  Terminator (3-prime TED) of KTI
gacacaagtgtgagagtactaaataaatgctttggttgtacgaaatcattacactaaataaaataatcaaagcttatatatgccttctaaggccgaatgcaaagaaattggttcttctcgttatctttgccactttactagtacgtattaattactacttaatcatcttgttacggctcattatatcc

SEQ ID NO: 18 - DNA, 325 bp, Glycine max (soybean)
  Terminator (3-prime TED) of LE
atgtgacagatcgaaggaagaaagtgtaataagacgactctcactactcgatcgctagtgattgtcattgttatatataataatgttatctttcacaacttatcgtaatgcattgtgaaactataacacatttaatcctacttgtcatatgataacactctccccatttaaaactcttgtcaatttaaagatataagattctttaaatgattaaaaaaaatatattataaattcaatcactcctactaataaattattaattaatatttattgattaaaaaaatacttatactaatttagtctgaatagaataattagattctag

SEQ ID NO: 19 - DNA, 213 bp, Glycine max (soybean)
  Terminator (3-prime TED) of P34
gccgtaaaggttcaatacaacgagtgcttgttttcttagggacaagcattgtacttatgtatgattctgtgtaaccatgagtcttccacgttgtactaatgtgaagggcaaaaataaaacacagaacaagttcgtttttctcaaataatgtgaaggtagaaaatggaaccatgcctcctctcttgcatgtgatttaaaatattagcagatggt

SEQ ID NO: 20 - DNA, 246 bp, Glycine max (soybean)
  Terminator (3-prime TED) of GBP
gaggtttagaacaatcaagaaaaggtgtgcatgtggctgaagatcacggggaatgtattaagcttcagagactctttaaattaaattttctgtattttgtgttatatgttactagttctttaaattagccagatggagtttatgtgtatctaaatgcagggatgctaatggaataaaatggccacttgtattgttagctatctcttatggtagcagaataagacgtaaactggttctttgctccaa

SEQ ID NO: 21 - DNA, 475 bp, Glycine max (soybean)
  Terminator (3-prime TED) of SMP
ttaaaacgtgatctatgatacaacaatattagtatatatagacgcatgcagtttatatagtatatattgtcatgttgtatgtttttacattttggtttgcttgtttacattctcttcaaaaaaaaaaaaatgtgtagtacgtgtaaggttttgaagattggttctaggctccgtgggaaccatttcaacaataaacattttgcgcgttcttgtacacgtagtgatgagaagagatgccttatgggcagtatcatctaaaacttattttcatccatcatagaatttggatctattggactggactgaactgaactgaatgatccttttttcttttttaatttcattcactaacaaatacataaaacaccagatattaacttagccagtatgaattttaactattttgtctaatgctatgacttatcactgtctgtatcatctttaattcttttttcatattatttatattaaataa

SEQ ID NO: 22 - DNA, 18 bp, Artificial Sequence
  ER retention signal sequence (retention tag encodes KDEL followed by 2 stop codons)
aaggatgaactttaatga

SEQ ID NO: 23 - protein, 4 aa, Artificial Sequence
  ER retention tag - KDEL
KDEL

SEQ ID NO: 24 - DNA, 21 bp, Artificial Sequence
  ER retention signal sequence (retention tag encodes KHDEL followed by 2 stop codons)
aagcatgatgaactttaatga

SEQ ID NO: 25 - protein, 5 aa, Artificial Sequence
  ER retention tag - KHDEL
KHDEL

SEQ ID NO: 26 - protein, 4 aa, Artificial Sequence
  ER retention signal sequence (retention tag encodes HDEL)
HDEL

SEQ ID NO: 27 - protein, 4 aa, Artificial Sequence
  ER retention tag - KEEL
KEEL

SEQ ID NO: 28 - protein, 6 aa, Artificial Sequence
  ER retention tag - SEKDEL
SEKDEL

SEQ ID NO: 29 - protein, 6 aa, Artificial Sequence
  ER retention tag - SEHDEL
SEHDEL

SEQ ID NO: 30 - DNA, 920 bp, Solanum tuberosum
  promoter/upstream regulatory region of Ubiquitin 3
ccaaagcacatacttatcgatttaaatttcatcgaagagattaatatcgaataatcatatacatactttaaatacataacaaattttaaatacatatatctggtatataattaattttttaaagtcatgaagtatgtatcaaatacacatatggaaaaaattaactattcataatttaaaaaatagaaaagatacatctagtgaaattaggtgcatgtatcaaatacattaggaaaagggcatatatcttgatctagataattaacgattttgatttatgtataatttccaaatgaaggtttatatctacttcagaaataacaatatacttttatcagaacattcaacaaagcaacaaccaactagagtgaaaaatacacattgttctctagacatacaaaattgagaaaagaatctcaaaatttagagaaacaaatctgaatttctagaagaaaaaaataattatgcactttgctattgctcgaaaaataaatgaaagaaattagacttttttaaaagatgttagactagatatactcaaaagctattaaaggagtaatattcttcttacattaagtattttagttacagtcctgtaattaaagacacattttagattgtatctaaacttaaatgtatctagaatacatatatttgaatgcatcatatacatgtatccgacacaccaattctcataaaaaacgtaatatcctaaactaatttatccttcaagtcaacttaagcccaatatacattttcatctctaaaggcccaagtggcacaaaatgtcaggcccaattacgaagaaaagggcttgtaaaaccctaataaagtggcactggcagagcttacactctcattccatcaacaaagaaaccctaaaagccgcagcgccactgatttctctcctccaggcgaag

SEQ ID NO: 31 - DNA, 178 bp, Solanum tuberosum
  terminator of Ubiquitin 3
ttgattttaatgtttagcaaatgtcttatcagttttctctttttgtcgaacggtaatttagagttttttttgctatatggattttcgtttttgatgtatgtgacaaccctcgggattgttgatttatttcaaaactaagagtttttgtcttattgttctcgtctattttggatatcaa

SEQ ID NO: 32 - DNA, 174 bp, Solanum tuberosum
  terminator of Ubiquitin monomer/ribosomal protein (Genbank Accession No. Z11669.1)
ttttaatgtttagcaaatgtcttatcagttttctctttttgtcgaacggtaatttagagtttttttttgctatatggattttcgtttttgatgtatgtgacaaccctcgggattgttgatttatttcaaaactaagagtttttgcttattgttctcgtctattttggatatcaa

SEQ ID NO: 33 - DNA, 1026 bp, Escherichia coli
  ORF of Hygromycin resistance gene (hph)
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggcattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 34 - DNA, 717 bp, Artificial Sequence
  Synthetic sequence of Green Fluorescent Protein (GFP) originally derived from Aequorea Victoria
atgttcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatactga

SEQ ID NO: 35 - DNA, 2583 bp, Aspergillus kawachii
  original CDS sequence of Beta glucosidase (Accession No. AB003470)
atgaggttcactttgattgaggcggtggctctcactgctgtctcgctggccagcgctgatgaattggcttactccccaccgtattacccatccccttgggccaatggccagggcgactgggcgcaggcataccagcgcgctgttgatattgtctcgcagatgacattggctgagaaggtcaatctgaccacaggaactggatgggaattggagctatgtgttggtcagactggcggggttccccgattgggagttccgggaatgtgtttacaggatagccctctgggcgttcgcgactccgactacaactctgctttcccttccggtatgaacgtggctgcaacctgggacaagaatctggcatacctccgcggcaaggctatgggtcaggaatttagtgacaagggtgccgatatccaattgggtccagctgccggccctctcggtagaagtcccgacggtggtcgtaactgggagggcttctcccccgacccggccctaagtggtgtgctctttgcagagaccatcaagggtatccaagatgctggtgtggtcgcgacggctaagcactacattgcctacgagcaagagcatttccgtcaggcgcctgaagcccaaggttatggatttaacatttccgagagtggaagcgcgaacctcgacgataagactatgcacgagctgtacctctggcccttcgcggatgccatccgtgcgggtgctggcgctgtgatgtgctcctacaaccagatcaacaacagctatggctgccagaacagctacactctgaacaagctgctcaaggccgagctgggtttccagggctttgtcatgagtgattgggcggctcaccatgctggtgtgagtggtgctttggcaggattggatatgtctatgccaggagacgtcgactacgacagtggtacgtcttactggggtacaaacctgaccgttagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactatgtgtcggagggaccgtacgagaaggtcaaccactacgtgaacgtgcaacgcaaccacagcgaactgatccgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgctcaagaacaagaatggtgtattcaccgccaccgataactgggctatcgatcagattgaggcgcttgctaagaccgccagtgtctctcttgtctttgtcaacgccgactctggcgagggttacatcaatgtcgacggaaacctgggtgaccgcaagaacctgaccctgtggaggaacggcgataatgtgatcaaggctgctgctagcaactgcaacaacaccattgttatcattcactctgtcggcccagtcttggttaacgaatggtacgacaaccccaatgttaccgctattctctggggtggtctgcccggtcaggagtctggcaactctcttgccgacgtcctctatggccgtgtcaaccccggtgccaagtcgccctttacctggggcaagactcgtgaggcctaccaagattacttggtcaccgagcccaacaacggcaatggagccccccaggaagacttcgtcgagggcgtcttcattgactaccgcggattcgacaagcgcaacgagaccccgatctacgagttcggctatggtctgagctacaccactttcaactactcgaaccttgaggtgcaggttctgagcgcccccgcgtacgagcctgcttcgggtgagactgaggcagcgccaacttttggagaggttggaaatgcgtcgaattacctctaccccgacggactgcagaaaatcaccaagttcatctacccctggctcaacagtaccgatctcgaggcatcttctggggatgctagctacggacaggactcctcggactatcttcccgagggagccaccgatggctctgcgcaaccgatcctgcctgctggtggcggtcctggcggcaaccctcgcctgtacgacgagctcatccgcgtgtcggtgaccatcaagaacaccggcaaggttgctggtgatgaagttccccaactgtatgtttcccttggcggccccaacgagcccaagatcgtgctgcgtcaattcgagcgcatcacgctgcagccgtcagaggagacgaagtggagcacgactctgacgcgccgtgaccttgcaaactggaatgttgagaagcaggactgggagattacgtcgtatcccaagatggtgtttgtcggaagctcctcgcggaagccgccgctccgggcgtctctgcctactgttcactaa

SEQ ID NO: 36 - protein, 860 aa, Aspergillus kawachii
  full length protein sequence of Beta glucosidase (Accession No. BAA19913)
MRFTLIEAVALTAVSLASADELAYSPPYYPSPWANGQGDWAQAYQRAVDIVSQMTLAEKVNLTTGTGWELELCVGQTGGVPRLGVPGMCLQDSPLGVRDSDYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRKNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKPPLRASLPTVH

SEQ ID NO: 37 - protein, 841 aa, Artificial Sequence
  partial sequence of Beta glucosidase (Accession No. BAA19913) with signal sequence (19 aa) deleted
DELAYSPPYYPSPWANGQGDWAQAYQRAVDIVSQMTLAEKVNLTTGTGWELELCVGQTGGVPRLGVPGMCLQDSPLGVRDSDYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRKNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKPPLRASLPTVH

SEQ ID NO: 38 - protein, 866 aa, Artificial Sequence
  Beta glucosidase GSF V1 - Beta glucosidase (Accession No. BAA19913) with signal sequence substituted and KDEL added
MKTNLFLFLIFSLLLSLSSAEDELAYSPPYYPSPWANGQGDWAQAYQRAVDIVSQMTLAEKVNLTTGTGWELELCVGQTGGVPRLGVPGMCLQDSPLGVRDSDYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRKNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKPPLRASLPTVHKDEL

SEQ ID NO: 39 - protein, 866 aa, Artificial Sequence
  Beta glucosidase GSF V2 - Beta glucosidase (Accession No. BAA19913) with signal sequence substituted and KDEL added, as well as 13 amino acid substitutions at positions 59, 110, 210, 320, 382, 475, 524, 549, 695, 700, 704, 715 and 852
MKTNLFLFLIFSLLLSLSSAEDELAYSPPYYPSPWANGQGDWAQAYQRAVDIVSQMTLDEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGFGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKHKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASDYLYPSGLQRITKFIYPWLNGTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVHKDEL

SEQ ID NO: 40 - protein, 860 aa, Aspergillus niger
  Beta glucosidase (SEQ ID NO.2 from US Pat. No. 7223902, also Accession No. ABT13410)
MRFTLIEAVALTAVSLASADELAYSPPYYPSPWANGQGDWAQAYQRAVDIVSQMTLDEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGFGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKHKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASDYLYPSGLLRITKFIYPWLNGTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 41 - DNA, 2586 bp, Aspergillus terreus NIH2624
  CDS of Beta-glucosidase I precursor (Accession No. XM_001212225)
atgaagctttccattttggaggcagcagctttgacagctgcctccgtagtcagcgcacaggacgatctcgcatactccccgccgtactacccttctccctgggccgatggccacggtgagtggtcgaacgcgtacaagcgcgctgtagatatcgtctctcagatgacattgacggagaaggtcaatctcaccaccggtactggatgggagttggagaggtgtgtcggtcagacgggcagtgtccctagactgggaatcccaagcctctgtctgcaggatagccctctgggtattcgcatgtcggactataactcggccttccctgcgggtattaacgttgcggccacctgggacaagaagcttgcctaccaacgcggcaaggcaatgggcgaggaattcagtgacaagggtattgatgttcagttgggccctgctgccggtcctcttggcaggtcccccgatggaggccgaaactgggagggcttctctcctgatcccgccctgactggtgtgttgttcgccgagacgatcaagggtatccaggacgccggagttattgctaccgcgaagcactacattctcaacgaacaagagcatttccgccaggtcggcgaagcccagggctatggcttcaacatcaccgaaaccgtgagctcaaatgtggatgacaagaccatgcacgagctgtatctctggcccttcgccgatgcggtgcgcgcgggcgtgggcgctgtgatgtgctcctacaaccagatcaacaacagctacggatgccaaaacagtttgaccctgaacaagctcttgaaagccgaactcggatttcagggatttgtcatgagtgactggagtgctcaccacagcggtgttggcgccgccttggctggtttggacatgtccatgccgggagatatcagtttcgacagcggcacttccttctatggcacgaacctgactgttggcgtcctcaacggcaccattccccagtggcgtgtggatgacatggccgtccggatcatggctgcctactacaaggttggccgcgaccgtctctggactcctcccaatttcagctcgtggactcgcgatgaatatggcttcgcgcacttcttcccttccgaaggcgcttatgaacgtgtcaatgaattcgtcaacgtgcagcgtgaccatgcccaggtgatccgtcggattggcgcggatagtgtcgtgctcttgaagaacgacggtgcccttcccttgacgggccaggagaagactgttggcattctgggcgaagacgccgggtcgaatccgaagggagcaaatggttgcagtgaccgtggctgtgacaagggtactctggccatggcttggggtagtggtactgccaacttcccttaccttgtgactcccgaacaggccattcagaacgaggttctgaagggccgtggaaatgtctttgccgtgacggacaactatgatacacagcagattgccgccgttgcctctcaatccacggtttcattggttttcgtgaacgcagacgccggtgaaggtttccttaatgtggacggaaacatgggtgatcgcaagaacctcaccctctggcagaacggagaggaagtgatcaagactgtcacggagcactgcaacaacaccgttgttgtgatccattcggtgggacctgttctcatcgatgagtggtatgcgcaccccaatgtcaccggcattctgtgggctggtctcccgggccaggagtctggcaacgccattgcggacgtgctgtacggccgcgtcaaccctggcggcaagaccccctttacctggggtaagacgcgcgcgtcctacggcgactacctcctcaccgagcccaacaacggcaacggtgctcctcaagacaacttcaacgagggcgtgtttattgactaccgtcgcttcgacaagtacaatgagacgcccatctacgagttcggtcatggtctgagctacacgacgtttgagctgtctggcctccaggtccagcttatcaacggatccagctatgttcccactacgggtcagacgagcgccgcccaggcatttggtaaagtcgaggacgcgtctagctacctgtaccctgagggactgaagaggatttccaagttcatctatccctggctgaactctaccgatcttaaagcgtctaccggcgatcctgaatacggagagcccaacttcgagtatattcctgaaggtgctaccgatggctctcctcagccccgtctgcctgccagcgggggtcctggcggcaaccccggtctctatgaggatctcttccaggtttctgtgaccatcaccaacaccggcaaggttgctggtgatgaggtgcctcagctgtatgtttcgctgggtggccccaacgagccgaagcgggtgctgcgcaagttcgagcgcctgcacatcgcccctggtcagcaaaaggtctggacgactaccctgaaccgccgtgacctagccaactgggatgtcgtggcccaggactggaagatcactccctatgctaagaccatctttgttggcacctcttcgcgcaagctgcctctcgctggtcgcttgccacgggtgcagtaa

SEQ ID NO: 42 - protein, 861 aa, Aspergillus terreus NIH2624
  Beta-glucosidase I precursor (translation of Accession No. XM_001212225)
MKLSILEAAALTAASVVSAQDDLAYSPPYYPSPWADGHGEWSNAYKRAVDIVSQMTLTEKVNLTTGTGWELERCVGQTGSVPRLGIPSLCLQDSPLGIRMSDYNSAFPAGINVAATWDKKLAYQRGKAMGEEFSDKGIDVQLGPAAGPLGRSPDGGRNWEGFSPDPALTGVLFAETIKGIQDAGVIATAKHYILNEQEHFRQVGEAQGYGFNITETVSSNVDDKTMHELYLWPFADAVRAGVGAVMCSYNQINNSYGCQNSLTLNKLLKAELGFQGFVMSDWSAHHSGVGAALAGLDMSMPGDISFDSGTSFYGTNLTVGVLNGTIPQWRVDDMAVRIMAAYYKVGRDRLWTPPNFSSWTRDEYGFAHFFPSEGAYERVNEFVNVQRDHAQVIRRIGADSVVLLKNDGALPLTGQEKTVGILGEDAGSNPKGANGCSDRGCDKGTLAMAWGSGTANFPYLVTPEQAIQNEVLKGRGNVFAVTDNYDTQQIAAVASQSTVSLVFVNADAGEGFLNVDGNMGDRKNLTLWQNGEEVIKTVTEHCNNTVVVIHSVGPVLIDEWYAHPNVTGILWAGLPGQESGNAIADVLYGRVNPGGKTPFTWGKTRASYGDYLLTEPNNGNGAPQDNFNEGVFIDYRRFDKYNETPIYEFGHGLSYTTFELSGLQVQLINGSSYVPTTGQTSAAQAFGKVEDASSYLYPEGLKRISKFIYPWLNSTDLKASTGDPEYGEPNFEYIPEGATDGSPQPRLPASGGPGGNPGLYEDLFQVSVTITNTGKVAGDEVPQLYVSLGGPNEPKRVLRKFERLHIAPGQQKVWTTTLNRRDLANWDVVAQDWKITPYAKTIFVGTSSRKLPLAGRLPRVQ

SEQ ID NO: 43 - protein, 513 aa, Trichoderma reesei
  Full length protein of Exoglucanase 1 (Accession No. P62694)
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 44 - protein, 521 aa, Artificial Sequence
  Modified exocellobiohydrolase I protein (ER signal replaced and KDEL sequence added to Accession No. P62694)
MKTNLFLFLIFSLLLSLSSAEQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCLKDEL

SEQ ID NO: 45 - protein, 471 aa, Trichoderma reesei
  Full length protein of Exoglucanase 2 (Accession No. P07987)
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 46 - protein, 472 aa, Artificial Sequence
  Modified Exoglucanase 2 (ER signal replaced and KDEL sequence added to Accession No. P07987)
MKTNLFLFLIFSLLLSLSSAEQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGPLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFLKDEL

SEQ ID NO: 47 - protein, 562 aa, Acidothermus cellulolyticus
  full length sequence of Endoglucanase E1 (Accession No. P54583)
MPRALRRVPGSRVMLRVGVVVAVLALVAALANLAVPRPARAAGGGYWHTSGREILDANNVPVRIAGINWFGFETCNYVVHGLWSRDYRSMLDQIKSLGYNTIRLPYSDDILKPGTMPNSINFYQMNQDLQGLTSLQVMDKIVAYAGQIGLRIILDRHRPDCSGQSALWYTSSVSEATWISDLQALAQRYKGNPTVVGFDLHNEPHDPACWGCGDPSIDWRLAAERAGNAVLSVNPNLLIFVEGVQSYNGDSYWWGGNLQGAGQYPVVLNVPNRLVYSAHDYATSVYPQTWFSDPTFPNNMPGIWNKNWGYLFNQNIAPVWLGEFGTTLQSTTDQTWLKTLVQYLRPTAQYGADSFQWTFWSWNPDSGDTGGILKDDWQTVDTVKDGYLAPIKSSIFDPVGASASPSSQPSPSVSPSPSPSPSASRTPTPTPTPTASPTPTLTPTATPTPTASPTPSPTAASGARCTASYQVNSDWGNGFTVTVAVTNSGSVATKTWTVSWTFGGNQTITNSWNAAVTQNGQSVTARNMSYNNVIQPGQNTTFGFQASYTGSNAAPTVACAAS

SEQ ID NO: 48 - DNA, 636 bp, Aspergillus niger
  CDS of Xylanase (Accession No. U39784)
atgaaggtcactgcggcttttgcaagtctcttgcttacggccttcgcggcccctgctccggagcctgttctggtgtcgcgaagtgccggtatcaactacgtgcagaactacaacggcaaccttggtgacttcacctacgacgagagtaccgggacattttccatgtactgggaggatggagtcagttccgacttcgtcgttggtttgggctggaccactggctcctctaaatctatcacctactctgcccaatacagcgcttctagctccagctcctacctggctgtctacggctgggtcaactctcctcaggccgaatactacatcgtcgaggattacggtgattacaacccttgcagctcggccacgagccttggtaccgtgtactctgatggaagcacctaccaagtctgcaccgacactcgacgaacgcggccatctatcacaggaacaagcacgttcacgcagtacttctccgttcgtgaaagtacacgcacatccggaacagtgactatcgccaaccatttcaatttctgggcgcagcatgggttcggcaatagcaacttcaattatcaggtcatggcggtggaggcatggaacggtgtcggcagtgccagtgtcacgatctcctcttaa

SEQ ID NO: 49 - protein, 211 aa, Aspergillus niger
  protein sequence of Xylanase (Accession No. AAA99065.1)
MKVTAAFASLLLTAFAAPAPEPVLVSRSAGINYVQNYNGNLGDFTYDESTGTFSMYWEDGVSSDFVVGLGWTTGSSKSITYSAQYSASSSSSYLAVYGWVNSPQAEYYIVEDYGDYNPCSSATSLGTVYSDGSTYQVCTDTRRTRPSITGTSTFTQYFSVRESTRTSGTVTIANHFNFWAQHGFGNSNFNYQVMAVEAWNGVGSASVTISS

SEQ ID NO: 50 - protein, 366 aa, Phanerochaete chrysosporium
  Ligninase 1508163A
MAFKQLFAAISLALSLSAANAAAVIEKRATCSNGKTVGDASCCAWFDVLDDIQQNLFHGGQCGAEAHESIRLVFHDSIAISPAMEAQGKFGGGGADGSIMIFDDIETAFHPNIGLDEIVKLQKPFVQKHGVTPGDFIAFAGAVALSNCPGAPQMNFFTGRAPATQPAPDGLVPEPFHTVDQIINRVNDAGEFDELELVWMLSAHSVAAVNDVDPTVQGLPFDSTPGIFDSQFFVETQLRGTAFPGSGGNQGEVESPLPGEIRIQSDHTIARDYRTACEWQSFVNNQSKLVDDFQFIFLALTQLGQDPNAMTDCSDVIPQSKPIPGNLPFSFFPAGKTIKDVEQACAETPFPTLTTLPGPETSVQRI

SEQ ID NO: 51 - protein, 102 aa, Trametes versicolor
  Ligninase manganese peroxidase (EC 1.11.1.13)
VAXPDGVNTATNAAXXQLFDGGECGEEVHESIARHXAIGVSNCPGAPQIGVSNXPGAPQLARDSRTAXEWQSLLIEXSELVPXPPPALSNADVEQAXAETPF

SEQ ID NO: 52 - protein, 371 aa, Phanerochaete chrysosporium (a basidiomycete)
  Lignin peroxidase (Accession No. P49012)
MAFKQLFAAITVALSLTAANAAVVKEKRATCANGKTVGDASCCAWFDVLDDIQANMFHGGQCGAEAHESIRLVFHDSIAISPAMEAKGKFGGGGADGSIMIFDTIETAFHPNIGLDEVVAMQKPFVQKHGVTPGDFIAFAGAVALSNCPGAPQMNFFTGRKPATQPAPDGLVPEPFHTVDQIIARVNDAGEFDELELVWMLSAHSVAAVNDVDPTVQGLPFDSTPGIFDSQFFVETQFRGTLFPGSGGNQGEVESGMAGEIRIQTDHTLARDSRTACEWQSFVGNQSKLVDDFQFIFLALTQLGQDPNAMTDCSDVIPLSKPIPGNGPFSFFPPGKSHSDIEQACAETPFPSLVTLPGPATSVARIPPHKA

SEQ ID NO: 53 - DNA, 134 bp, Glycine max (soybean)
  sequence for beta-conglycinin alpha subunit (BCS) (genbank Accession No. AB237643.1)
cctacagacttcaatctggtgatgccctgagagtcccctcaggaaccacatactatgtggtcaaccctgacaacaacgaaaatctcagattaataacactcgccatacccgttaacaagcctggtagatttgag

SEQ ID NO: 54 - DNA, 1195 bp, Artificial Sequence
  complete RNAi cassette to produce BCS
gcggccgcctcgagcctacagacttcaatctggtgatgccctgagagtcccctcaggaaccacatactatgtggtcaaccctgacaacaacgaaaatctcagattaataacactcgccatacccgttaacaagcctggtagatttgaggatatcgagctcgggctgtttctcttctcgtcacactcacaatagggtggcctatgtatttagccttcaatgtctctggtagaccctatgatagttttgcaagccactaccacccttatgctcccatatattctaaccgtgaaagcttactagtctagtaccccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgtaaagctttcacggttagaatatatgggagcataagggtggtagtggcttgcaaaactatcatagggtctaccagagacattgaaggctaaatacataggccaccctattgtgagtgtgacgagaagagaaacagcccgagctcgatatcctcaaatctaccaggcttgttaacgggtatggcgagtgttattaatctgagattttcgttgttgtcagggttgaccacatagtatgtggttcctgaggggactctcagggcatcaccagattgaagtctgtaggctcgagtctagagcggccgc

SEQ ID NO: 55 - DNA, 332 bp, Artificial Sequence
  Glycinin RNAi fragment from glycinin A1bB2 (Genbank Accession No. AB030495)
cattgacgagaccatttgcacaatgggacttcgccacaacataggccagacttcatcacctgacatcttcaaccctcaagctggtagcatcacaaccgctaccagcctcgacttcccagccctctcgtggctcaaactcagtgcccagtttggatcactccgcaagaatgctatgttcgtgccacactacaacctgaacgcaaacagcataatatacgcattgaatggacgggcattggtacaagtggtgaattgcaatggtgagagagtgtttgatggagagctgcaagagggacaggtgttaactgtgccacaaaactttgcggtggctg

SEQ ID NO: 56 - DNA, 1591 bp, Artificial Sequence
  complete RNAi cassette to produce SP-
gcggccgcctcgagcattgacgagaccatttgcacaatgggacttcgccacaacataggccagacttcatcacctgacatcttcaaccctcaagctggtagcatcacaaccgctaccagcctcgacttcccagccctctcgtggctcaaactcagtgcccagtttggatcactccgcaagaatgctatgttcgtgccacactacaacctgaacgcaaacagcataatatacgcattgaatggacgggcattggtacaagtggtgaattgcaatggtgagagagtgtttgatggagagctgcaagagggacaggtgttaactgtgccacaaaactttgcggtggctggatatcgagctcgggctgtttctcttctcgtcacactcacaatagggtggcctatgtatttagccttcaatgtctctggtagaccctatgatagttttgcaagccactaccacccttatgctcccatatattctaaccgtgaaagcttactagtctagtaccccaattggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgtaaagctttcacggttagaatatatgggagcataagggtggtagtggcttgcaaaactatcatagggtctaccagagacattgaaggctaaatacataggccaccctattgtgagtgtgacgagaagagaaacagcccgagctcgatatccagccaccgcaaagttttgtggcacagttaacacctgtccctcttgcagctctccatcaaacactctctcaccattgcaattcaccacttgtaccaatgcccgtccattcaatgcgtatattatgctgtttgcgttcaggttgtagtgtggcacgaacatagcattcttgcggagtgatccaaactgggcactgagtttgagccacgagagggctgggaagtcgaggctggtagcggttgtgatgctaccagcttgagggttgaagatgtcaggtgatgaagtctggcctatgttgtggcgaagtcccattgtgcaaatggtctcgtcaatgctcgagtctagagcggccgc

SEQ ID NO: 57 - DNA, 130 bp, Artificial Sequence
  FAD2-1 gene (Genbank Accession No. AB188250)
gggctgtttctcttctcgtcacactcacaatagggtggcctatgtatttagccttcaatgtctctggtagaccctatgatagttttgcaagccactaccacccttatgctcccatatattctaaccgtga